(19) United States
(12) Patent Application Publication
(10) Pub. No.: US 2017/0283834 A1
(43) Pub. Date: Oct. 5, 2017

(54) Processes for Producing A Fermentation Product Using A Fermenting Organism

(71) Applicant: Novozymes A/S, Bagsvaerd (DK)

(72) Inventors: Jennifer Headman, Franklinton, NC (US); Eric Allain, Boone, NC (US); Jeremy Saunders, Raleigh, NC (US)

(21) Appl. No.: 15/508,398

(22) PCT Filed: Aug. 31, 2015
(86) PCT No.: PCT/US2015/047690

§ 371 Date: Mar. 2, 2017

Related U.S. Application Data
(60) Provisional application No. 62/055,299, filed on Sep. 25, 2014.
(60) Provisional application No. 62/044,571, filed on Sep. 2, 2014.

(51) Int. Cl. C12P 7/06

(52) U.S. Cl. CPC C12P 7/06; C12Y 302/01001; C12Y 302/01003; C12Y 304/24039; C12Y 304/23018

(57) ABSTRACT

The present invention relates to processes for producing a fermentation product, such as ethanol, from starch-containing material; wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.

### FIELD OF THE INVENTION

The present invention relates to processes for producing a fermentation product, such as ethanol, from starch-containing material using a fermenting organism.

### REFERENCE TO A SEQUENCE LISTING

This application contains a Sequence Listing in computer readable form. The computer readable form is incorporated herein by reference.

### BACKGROUND OF THE INVENTION

Production of fermentation products, such as ethanol, from starch-containing material is well-known in the art. The most commonly industrially used commercial process, often referred to as a “conventional process”, includes liquefying gelatinized starch at high temperature (around 85° C.) using typically a bacterial alpha-amylase, followed by simultaneous saccharification and fermentation carried out under anaerobic conditions in the presence of a glucoamylase and a fermenting organism, typically a Saccharomyces cerevisae yeast when producing ethanol.

Another type of process is also used commercially today. Starch is converted into sugars by enzymes at temperatures below the initial gelatinization temperature of the starch in question and converted into ethanol by yeast, typically derived from Saccharomyces cerevisiae. This type of process is referred to as a raw starch hydrolysis (RSH) process, or alternatively a “one-step process” or “no cook” process.

Despite significant improvements over the past decade there is still a desire and need for providing improved processes of producing fermentation products from starch-containing material in a more cost efficient way.

### SUMMARY OF THE INVENTION

The present invention relates to processes for producing fermentation products, such as ethanol, from starch-containing material.

In the first aspect the invention relates to processes of producing fermentation productsfrom starch-containing material comprising the steps of:

i) liquefying the starch-containing material at a temperature above the initial gelatinization temperature using an alpha-amylase;
ii) saccharifying using a glucoamylase;
iii) fermenting using a fermenting organism;
wherein an acid having a pKa in the range from 3.75 to 5.75 is present and/or added in fermentation so that the acid concentration in fermentation is maintained between above 0 (zero) and 100 mmoles/L fermentation medium and wherein the acid is added before the exponential growth phase of the fermenting organism.

In a preferred embodiment the acid concentration in fermentation is maintained between 10 and 100 mmoles/L fermentation medium.

In another embodiment the acid concentration in fermentation is maintained between 5 and 80 mmoles/L fermentation medium.

The term “pKa” means the dissociation constant (K) and defines the ratio of the concentrations of the dissociated ions and the undissociated acid.

In a preferred embodiment the fermenting organism is yeast, preferably derived from a strain of Saccharomyces, such as a strain of Saccharomyces cerevisiae.

Steps ii) and iii) are carried out either sequentially or simultaneously. In a preferred embodiment steps ii) and iii) are carried out simultaneously, i.e., simultaneous saccharification and fermentation (SSF).

According to the process of the invention liquefaction in step i) is carried out by subjecting starch-containing material at a temperature above the initial gelatinization temperature, typically between 80-90° C., using an alpha-amylase. The pH in liquefaction is between 4-7, preferably between 4.5 and 6.0, such as between 4.8 and 5.8. Examples of alpha-amylase can be found below in the “Alpha-Amylase Present and/or Added During Liquefaction”-section. In an embodiment the alpha-amylase is a bacterial alpha-amylase. In a preferred embodiment the alpha-amylase is from the genus Bacillus, such as a strain of Bacillus stearothermophilus, in particular a variant of Bacillus stearothermophilus alpha-amylase, such as the one shown in SEQ ID NO: 3 in WO 99/019467 or SEQ ID NO: 1 herein. Examples of suitable thermostable Bacillus stearothermophilus alpha-amylase variants can be found below in the “Thermostable Alpha-Amylase”-section and include one from the following group of Bacillus stearothermophilus alpha-amylase variants with the following mutations:

I181*+G182*+N193F+E129V+K177L+R179E;

I181*+G182*+N193F+V59A+Q89R+E129V+K177L+R179E+H208Y+K220P+N224L+Q254S;

I181*+G182*+N193F+V59A+Q89R+E129V+K177L+R179E+Q254S+M284V;

I181*+G182*+N193F+V59A+E129V+K177L+R179E+Q254S+M284V; and

I181*+G182*+N193F+E129V+K177L+R179E+K220P+N224L+S242Q+Q254S (using SEQ ID NO: 1 for numbering).

Examples of other suitable Bacillus stearothermophilus alpha-amylases having increased thermostability compared to a reference alpha-amylase (Bacillus stearothermophilus alpha-amylase with the mutations I181*+G182*+N193F truncated to 491 amino acids) at pH 4.5 and 5.5, 0.12 mM CaCl₂ can be found in WO 2011/082425 hereby incorporated by reference. See also Example 1 below. Liquefaction in step i) may be carried out using a combination of alpha-amylase and protease. The protease may be a protease having a thermostability value of more than 20% determined as Relative Activity at 80° C./70° C. Examples of suitable proteases are described below in the section “Protease Present and/or Added In Liquefaction”.

The protease may be of fungal origin, such as of filamentous fungus origin. Specific examples of suitable fungal proteases are protease variants of metallo protease derived from a strain of the genus Thermoascus, preferably a strain of Thermoascus aurantiacus, especially the strain Thermoascus aurantiacus CGMCC No. 0670 disclosed as the mature part of SEQ ID NO. 2 disclosed in WO 2003/048353 or the mature part of SEQ ID NO: 1 in WO 2010/008841 or SEQ ID NO: 3 herein with the following mutations:

D79L+S87P+A112P+D142L:

D79L+S87P+D142L; or

A27K+D79L+Y82F+S87G+D104P+A112P+A126V+D142L.

Examples of other suitable protease variants can be found in WO 2011/072191 hereby incorporated by reference. See also Example 2 below. Suitable proteases also include bacterial proteases. A suitable bacterial protease may be derived from a strain of Pyrococcus, preferably a strain of Pyrococcus furiosus. In a preferred embodiment the protease is the one shown in SEQ ID NO: 1 in U.S. Pat. No. 6,358,726 or SEQ ID NO: 13 herein. In an embodiment of the invention the alpha-amylase and/or the protease added in the liquefaction step i) is further combined with a glucoamylase. Thus, a glucoamylase may also be present and/or added during liquefaction step i). The glucoamylase is preferably thermostable. That means that the glucoamylase has a heat stability at 85° C., pH 5.3, of at least 20%, such as at least 30%, preferably at least 35% determined as described in Example 4 (heat stability). In an embodiment the glucoamylase present and/or added in liquefaction has a relative activity pH optimum at pH 5.0 of at least 90%, preferably at least 95%, preferably at least 97%. In an embodiment the glucoamylase has a pH stability at pH 5.0 of at least at least 80%, at least 85%, at least 90% determined as described in Example 4 (pH optimum).

A suitable glucoamylase present and/or added in liquefaction step i) may according to the invention be derived from a strain of the genus Penicillium, especially a strain of Penicillium oxalicum disclosed as SEQ ID NO: 2 in WO 2011/127802 or SEQ ID NOs: 9 or 14 herein. In a preferred embodiment the glucoamylase is a variant of the Penicillium oxalicum glucoamylase shown in SEQ ID NO: 2 in WO 2011/127802 having a K79V substitution (using the mature sequence shown in SEQ ID NO: 14 herein for numbering), such as a variant disclosed in WO 2013/053801. In a preferred embodiment the Penicillium oxalicum glucoamylase has a K79V substitution (using SEQ ID NO: 14 for numbering) and further one of the following set of substitutions:

P11F+T65A+Q327F;

P2N+P4S+P11F+T65A+Q327F (using SEQ ID NO: 14 for numbering).

Examples of other suitable Penicillium oxalicum glucoamylase variants can be found in WO 2013/053801 incorporated by reference. See also Example 15 below.

In an embodiment the glucoamylase, such as a Penicillium oxalicum glucoamylase variant, used in liquefaction has a thermostability determined as DSC Td at pH 4.0 as described in Example 15 of at least 70° C., preferably at least 75° C., such as at least 80° C., such as at least 81° C., such as at least 82° C., such as at least 83° C., such as at least 84° C., such as at least 85° C., such as at least 86° C., such as at least 87%, such as at least 88° C., such as at least 89° C., such as at least 90° C. In an embodiment the glucoamylase, such as a Penicillium oxalicum glucoamylase variant has a thermostability determined as DSC Td at pH 4.0 as described in Example 15 in the range between 70° C. and 95° C., such as between 80° C. and 90° C.

In an embodiment the glucoamylase, such as a Penicillium oxalicum glucoamylase variant, used in liquefaction has a thermostability determined as DSC Td at pH 4.8 as described in Example 15 of at least 70° C., preferably at least 75° C., such as at least 80° C., such as at least 81° C., such as at least 82° C., such as at least 83° C., such as at least 84° C., such as at least 85° C., such as at least 86° C., such as at least 87%, such as at least 88° C., such as at least 89° C., such as at least 90° C., such as at least 91° C. In an embodiment the glucoamylase, such as a Penicillium oxalicum glucoamylase variant has a thermostability determined as DSC Td at pH 4.8 as described in Example 15 in the range between 70° C. and 95° C., such as between 80° C. and 90° C.

In an embodiment the glucoamylase, such as a Penicillium oxalicum glucoamylase variant, used in liquefaction has a residual activity determined as described in Example 16 of at least 100% such as at least 105%, such as at least 110%, such as at least 115%, such as at least 120%, such as at least 125%. In an embodiment the glucoamylase, such as a Penicillium oxalicum glucoamylase variant has a thermostability determined as residual activity as described in Example 16 in the range between 100% and 130%.

Further, according to the process of the invention also a pullulanase may be present in liquefaction in combination with an alpha-amylase, a protease and/or a glucoamylase.

According to the process of the invention a glucoamylase may be present and/or added in saccharification and/or fermentation or simultaneous saccharification and fermentation. The glucoamylase may not be the same as the thermostable glucoamylase used in liquefaction.

In an embodiment the glucoamylase present and/or added in saccharification and/or fermentation is of fungal origin, such as of filamentous fungus origin. In a preferred embodiment the glucoamylase is derived from a strain of Aspergillus, preferably Aspergillus niger, Aspergillus awamori, or Aspergillus oryzae; or a strain of Trichoderma, preferably Trichoderma reesei; or a strain of Talaromyces, preferably Talaromyces emersonii, or a strain of Pycnoporus, or a strain of Gloephyllum, such as Gloephyllum serpiarium or Gloephyllum trabeum, or a strain of the Nigrofomes.

In an embodiment the glucoamylase is derived from Talaromyces emersonii, such as the one shown in SEQ ID NO: 19 herein. In another embodiment the glucoamylase present and/or added in saccharification and/or fermentation is derived from Gloephyllum serpiarium, such as the one shown in SEQ ID NO: 15 herein. In another embodiment the glucoamylase present and/or added in saccharification and/or fermentation is derived from Gloeophyllum trabeum such as the one shown in SEQ ID NO: 17 herein.

In a preferred embodiment the glucoamylase is present and/or added in saccharification and/or fermentation in combination with an alpha-amylase. The alpha-amylase may be of fungal or bacterial origin.

The alpha-amylase present added in saccharification and/or fermentation in combination with a glucoamylase may be derived from a strain of the genus Rhizomucor, preferably a strain the Rhizomucor pusillus, such as the one shown in SEQ ID NO: 3 in WO 2013/006756, such as a Rhizomucor pusillus alpha-amylase hybrid having a linker and starch binding domain, in particular an Aspergillus niger linker and starch-bonding domain, such as the one shown in SEQ ID NO: 16 herein.

In a preferred embodiment the alpha-amylase is derived from a strain of Rhizomucor pusillus, preferably with an Aspergillus niger glucoamylase linker and starch-binding domain (SBD), preferably the one disclosed as SEQ ID NO: 16 herein, preferably having one or more of the following substitutions: G128D, D143N, preferably G128D+D143N (using SEQ ID NO: 16 herein for numering).

In an embodiment the invention relates to processes for producing fermentation products, such as especially ethanol, from starch-containing material comprising the steps of:

i) liquefying the starch-containing material at a temperature above the initial gelatinization temperature, such as between 80-90° C., using an alpha-amylase derived from Bacillus stearothermophilus;
ii) saccharifying using a glucoamylase;
iii) fermenting using a fermenting organism;
wherein an acid having a pKa in the range from 3.75 to 5.75 is present and/or added in fermentation so that the acid concentration in fermentation is maintained between above 0 (zero) and 100 mmoles/L fermentation medium and wherein the acid is added before the exponential growth phase of the fermenting organism.

In a preferred embodiment the acid concentration in fermentation is maintained between 10 and 100 mmoles/L fermentation medium.

In a preferred embodiment the fermenting organism is yeast.

In a specific embodiment the fermenting organism is ETHANOL RED™ (“ER”) (Fermentis).

In an embodiment of the invention a cellulolytic composition is present and/or added in saccharification, fermentation or simultaneous saccharification and fermentation (SSF). Examples of such compositions can be found in the “Cellulolytic Composition present and/or added during Saccharification and/or Fermentation”-section below. In a preferred embodiment the cellulolytic composition is present and/or added together with a glucoamylase, suchh as one disclosed in the “Glucoamylase Present And/Or Added in Saccharification and/or Fermentation”-section below.

In another aspect the invention relates to processes for producing a fermentation product from starch-containing material comprising the steps of:

(i) saccharifying the starch-containing material at a temperature below the initial gelatinization temperature
(ii) fermenting using a fermenting organism;
wherein saccharification and/or fermentation is done in the presence of the following enzymes: glucoamylase and alpha-amylase, and optionally protease; and wherein an acid having a pKa in the range from 3.75 to 5.75 is present and/or added in fermentation so that the acid concentration in fermentation is maintained between above 0 (zero) and 100 mmoles/L fermentation medium and wherein the acid is added before the exponential growth phase of the fermenting organism.

In a preferred embodiment saccharification and fermentation are carried out simultaneosly (one step process).

### BRIEF DESCRIPTION OF THE FIGURES

FIG. 1 shows 48 and 54 hours ethanol titers over a range of 5 and 120 mM acetate additions.

FIG. 2 shows 48 and 54 hours glycerol titers over a range of added acetate concentrations.

FIG. 3 shows ethanol titers in response to benzoic acid concentratuibs between 0.1 to 0.8 mM.

FIG. 4 shows the ethanol titers when adding propionic acid at pH 3.8.

FIG. 5 shows the ethanol titers when adding propionic acid at pH 5.

FIG. 6 shows the ethanol titers when adding formic acid at pH 3.8.

FIG. 7 shows the ethanol titers when adding formic acid at pH 5.

### DETAILED DESCRIPTION OF THE INVENTION

### Conventional-Type Process

In this aspect the present invention relates to producing fermentation products, such as especially ethanol, from starch-containing material in a process including liquefaction, saccharification and fermentation. In a preferred embodiment fermentable sugars generated during saccharification are converted to ethanol during fermentation by yeast, especially Saccharomyces cerevisiae yeast.

The inventors have surprisingly found that addition of a (weak) acid with a pKa in the range from 3.75-5.75) or it's conjugate base (i.e. acetic acid/acetate) can improve overall ethanol yield. This is unexpected since some weak acids, including acetic acid (pKa=4.75), can inhibit growth. It is believed that weak acids are able to decouple ATP production and biomass growth by forcing the cell to use ATP driven proton pumping to expel H⁺ that the protonated weak acid can carry into the fermenting organism cell, such as yeast cell. Also, the fermenting organism cell likely spends ATP to re-transport the weak acid back out (where it can become protonated again and the cycle can continue). This ATP drain means the cell has to make more ATP to generate a given amount of biomass thus more ethanol produced per cell.

In the first aspect the invention relates to processes for producing fermentation products, such as ethanol, from starch-containing material comprising the steps of:
i) liquefying the starch-containing material at a temperature above the initial gelatinization temperature using an alpha-amylase;
ii) ii) saccharifying using a glucoamylase;
iii) iii) fermenting using a fermenting organism;
   wherein an acid having a pKa in the range from 3.75 to 5.75 is present and/or added in fermentation so that the acid concentration in fermentation is maintained between above 0 (zero) and 100 mmoles/L fermentation medium and wherein the acid is added before the exponential growth phase of the fermenting organism.

In a preferred embodiment the acid concentration is maintained between 10 and 100 mmoles/L fermentation medium.

According to the process of the invention the fermenting organism may be any fermenting organism, such as especially yeast, such as a strain of the genus Saccharomyces, especially a strain of the species Saccharomyces cerevisiae. The Saccharomyces cerevisiae yeast may be a baker's yeast (Saccharomyces cerevisiae), such as ETHANOL RED™ (Fermentis) which is commonly used for large scale commercial production of ethanol.

In an embodiment the acid is a weak acid selected from the group of: acetic acid, benzoic acid, propionic acid, formic acid, sorbic acid and succinic acid. In a preferred embodiment the (weak) acid has a pKa in the range from 4.0-5.0. In an embodiment a combination of weak acids are used. The dosing of acid depends to some degree on the acid in question. Too low dosages/concentrations of acid may have no effect of the fermentation product, such as ethanol, yield. Too high acid dosages/concentrations) may inhibit the fermenting organism, such as yeast, growth. When the acid in question is acetic acid (pKa=4.75) the concentration in fermentation is according to the invention between 20-80 mmoles/L fermentation medium). In a preferred embodiment the acid is hydrophobic when protonated.

In an embodiment the acid is added during lag phase.

Steps ii) and iii) are carried out either sequentially or simultaneously. In a preferred embodiment steps ii) and iii) are carried out simultaneously.

### Liquefaction Step i)

According to the process of the invention liquefaction in step i) may be carried out by subjecting starch-containing material at a temperature above the initial gelatinization temperature to an alpha-amylase and optionally a protease, and/or a glucoamylase. Other enzymes such as a pullulanase and phytase may also be present and/or added in liquefaction.

Liquefaction step i) may be carried out for 0.5-5 hours, such as 1-3 hours, such as typically around 2 hours.

The term “initial gelatinization temperature” means the lowest temperature at which gelatinization of the starch-containing material commences. In general, starch heated in water begins to gelatinize between about 50° C. and 75° C.; the exact temperature of gelatinization depends on the specific starch and can readily be determined by the skilled artisan. Thus, the initial gelatinization temperature may vary according to the plant species, to the particular variety of the plant species as well as with the growth conditions. In the context of this invention the initial gelatinization temperature of a given starch-containing material may be determined as the temperature at which birefringence is lost in 5% of the starch granules using the method described by Gorinstein and Lii, 1992, Starch/Stärke 44(12): 461-466.

According to the invention liquefaction is typically carried out at a temperature in the range from 70-100° C. In an embodiment the temperature in liquefaction is between 75-95° C., such as between 75-90° C., preferably between 80-90° C., such as 82-88° C., such as around 85° C.

According to the invention a jet-cooking step may be carried out prior to liquefaction in step i). The jet-cooking may be carried out at a temperature between 110-145° C., preferably 120-140° C., such as 125-135° C., preferably around 130° C. for about 1-15 minutes, preferably for about 3-10 minutes, especially around about 5 minutes.

The pH during liquefaction may be between 4-7, such as between pH 4.5-6,5, such as between pH 5.0-6.5, such as between pH 5.0-6.0, such as between pH 5.2-6.2, such as around 5.2, such as around 5.4, such as around 5.6, such as around 5.8.

In an embodiment, the process of the invention further comprises, prior to the step i), the steps of:

a) reducing the particle size of the starch-containing material, preferably by dry milling;

b) forming a slurry comprising the starch-containing material and water.

The starch-containing starting material, such as whole grains, may be reduced in particle size, e.g., by milling, in order to open up the structure, to increase surface area, and allowing for further processing. Generally there are two types of processes: wet and dry milling. In dry milling whole kernels are milled and used. Wet milling gives a good separation of germ and meal (starch granules and protein). Wet milling is often applied at locations where the starch hydrolysate is used in production of, e.g., syrups. Both dry milling and wet milling are well known in the art of starch processing. According to the present invention dry milling is preferred.

In an embodiment the particle size is reduced to between 0.05 to 3.0 mm, preferably 0.1-0.5 mm, or so that at least 30%, preferably at least 50%, more preferably at least 70%, even more preferably at least 90% of the starch-containing material fit through a sieve with a 0.05 to 3.0 mm screen, preferably 0.1-0.5 mm screen. In another embodiment at least 50%, preferably at least 70%, more preferably at least 80%, especially at least 90% of the starch-containing material fit through a sieve with #6 screen.

The aqueous slurry may contain from 10-55 w/w-% dry solids (DS), preferably 25-45 w/w-% dry solids (DS), more preferably 30-40 w/w-% dry solids (DS) of starch-containing material.

The alpha-amylase, optionally a protease, optionally a glucoamylase may initially be added to the aqueous slurry to initiate liquefaction (thinning). In an embodiment only a portion of the enzymes (e.g., about ⅓) is added to the aqueous slurry, while the rest of the enzymes (e.g., about ⅔) are added during liquefaction step i).

A non-exhaustive list of examples of alpha-amylases can be found below in the “Alpha-Amylase Present and/or Added In Liquefaction”-section. In an embodiment the alpha-amylase is a bacterial alpha-amylase. Bacterial alpha-amylases are typically thermostable. In a preferred embodiment the alpha-amylase is from the genus Bacillus, such as a strain of Bacillus stearothermophilus, in particular a variant of a Bacillus stearothermophilus alpha-amylase, such as the one shown in SEQ ID NO: 3 in WO 99/019467 or SEQ ID NO: 1 herein.

In an embodiment the alpha-amylase has an improved stability compared to a reference alpha-amylase (Bacillus stearothermophilus alpha-amylase with the mutations I181*+G182*+N193F truncated to around 491 amino acids (using SEQ ID NO: 1 herein for numbering) determined by incubating the reference alpha-amylase and variants at pH 4.5 and 5.5 and temperatures of 75° C. and 85° C. with 0.12 mM CaCl₂ followed by residual activity determination using the EnzChek® substrate (EnzChek® Ultra Amylase assay kit, E33651, Molecular Probes). This is described in Example 1.

Examples of suitable Bacillus stearothermophilus alpha-amylase variants can be found below in the “Thermostable Alpha-Amylase”-section and include one from the following group of Bacillus stearothermophilus alpha-amylase variants with the following mutations: I181*+G182*, an optionally substitution N193F, and additionally the following substitutions
E129V+K177L+R179E;
V59A+Q89R+E129V+K177L+R179E+H208Y+K220P+N224L+Q254S;
V59A+Q89R+E129V+K177L+R179E+Q254S+M284V;
V59A+E129V+K177L+R179E+Q254S+M284V; and
E129V+K177L+R179E+K220P+N224L+S242Q+Q254S (using SEQ ID NO: 1 herein for numbering).

Examples of other suitable Bacillus stearothermophilus alpha-amylases having increased thermostability compared to a reference alpha-amylase (Bacillus stearothermophilus alpha-amylase with the mutations I181*+G182*+N193F, truncated to be 491 amino acids long) at pH 4.5 and 5.5, 0.12 mM CaCl₂ can be found in WO 2011/082425 hereby incorporated by reference. (See also Example 1 below)

According to the invention liquefaction step i) may be carried out using a combination of alpha-amylase and protease. The protease may be a protease having a thermostability value of more than 20% determined as Relative Activity at 80° C./70° C. determined as described in Example 1 (Relative Activity). Examples of suitable proteases are described below in the section “Protease Present and/or Added In Liquefaction”.

The protease may be of fungal origin, such as of filamentous fungus origin. Specific examples of suitable fungal proteases are protease variants of metallo protease derived from a strain of the genus Thermoascus, preferably a strain of Thermoascus aurantiacus, especially the strain Thermoascus aurantiacus CGMCC No. 0670 disclosed as the mature part of SEQ ID NO. 2 disclosed in WO 2003/048353 or the mature part of SEQ ID NO: 1 in WO 2010/008841 or SEQ ID NO: 3 herein with the following mutations:
D79L+S87P+A112P+D142L:
D79L+S87P+D142L; or
A27K+D79L+Y82F+S87G+D104P+A112P+A126V+D142L.
   More examples of suitable variants of the Thermoascus aurantiacus protease can be found in WO 2011/072191 hereby incorporated by reference. See also Example 2 below.

Suitable proteases also include bacterial proteases. A suitable bacterial protease may be derived from a strain of Pyrococcus, preferably a strain of Pyrococcus furiosus. In a preferred embodiment the protease is the one shown in SEQ ID NO: 1 in U.S. Pat. No. 6,358,726 or SEQ ID NO: 13 herein.

In an embodiment of the invention the alpha-amylase and/or protease, added in the liquefaction step i), is/are further combined with a glucoamylase. Thus, a glucoamylase may also be present and/or added during liquefaction step i). The glucoamylase is preferably thermostable. This means that the glucoamylase has a heat stability at 85° C., pH 5.3, of at least 20%, such as at least 30%, preferably at least 35% determined as described in Example 4 (heat stability). In an embodiment the glucoamylase present and/or added in liquefaction has a relative activity pH optimum at pH 5.0 of at least 90%, preferably at least 95%, preferably at least 97%. In an embodiment the glucoamylase has a pH stability at pH 5.0 of at least at least 80%, at least 85%, at least 90% determined as described in Example 4 (pH stability).

A suitable glucoamylase present and/or added in liquefaction step i) may according to the invention be derived from a strain of the genus Penicillium, especially a strain of Penicillium oxalicum disclosed as SEQ ID NO: 2 in WO 2011/127802 or SEQ ID NOs: 9 or 14 herein. In a preferred embodiment the glucoamylase is a variant of the Penicillium oxalicum glucoamylase shown in SEQ ID NO: 2 in WO 2011/127802 having a K79V substitution (using the mature sequence shown in SEQ ID NO: 14 herein for numbering), such as a variant disclosed in WO 2013/053801. In a preferred embodiment the Penicillium oxalicum glucoamylase has a K79V substitution (using SEQ ID NO: 14 herein for numbering) and further one of the following:

P11F+T65A+Q327F;

P2N+P4S+P11F+T65A+Q327F (using SEQ ID NO: 14 herein for numbering).

Examples of other suitable Penicillium oxalicum glucoamylase variants can be found in WO 2013/053801 incorporated by reference (see also Examples 10-16 below, such as the Penicillium oxalicum glucoamylase variants in Table 15).

Further, according to the process of the invention also a pullulanase may be present during liquefaction in combination with an alpha-amylase, a protease and/or a glucoamylase.

### Saccharification and Fermentation

A glucoamylase is present and/or added in saccharification step ii) and/or fermentation step iii) or simultaneous saccharification and fermentation (SSF). The glucoamylase added in saccharification step ii) and/or fermentation step iii) or simultaneous saccharification and fermentation (SSF) is typically different from the glucoamylase, optionally added in liquefaction step i). In a preferred embodiment the glucoamylase is added together with a fungal alpha-amylase. Examples of glucoamylases can be found in the “Glucoamylases Present and/or Added In Saccharification and/or Fermentation”-section below.

When doing sequential saccharification and fermentation, saccharification step ii) may be carried out at conditions well-known in the art. For instance, the saccharification step ii) may last up to from about 24 to about 72 hours. In an embodiment pre-saccharification is done. Pre-saccharification is typically done for 40-90 minutes at a temperature between 30-65° C., typically about around 60° C. Pre-saccharification is in an embodiment followed by saccharification during fermentation in simultaneous saccharification and fermentation (SSF). Saccharification is typically carried out at temperatures from 20-75° C., preferably from 40-70° C., typically around 60° C., and at a pH between 4 and 5, normally at about pH 4.5.

Simultaneous saccharification and fermentation (“SSF”) is widely used in industrial scale fermentation product production processes, especially ethanol production processes. When doing SSF the saccharification step ii) and the fermentation step iii) are carried out simultaneously. There is no holding stage for the saccharification, meaning that a fermenting organism, such as yeast, and enzyme(s), may be added together. However, it is also contemplated to add the fermenting organism and enzyme(s) separately. SSF is according to the invention typically carried out at a temperature from 25° C. to 40° C., such as from 28° C. to 35° C., such as from 30° C. to 34° C., preferably around about 32° C. In an embodiment fermentation is ongoing for 6 to 120 hours, in particular 24 to 96 hours. In an embodiment the pH is between 4-5.

In an embodiment of the invention a cellulolytic composition is present and/or added in saccharification, fermentation, or simultaneous saccharification and fermentation (SSF). Examples of such cellulolytic compositions can be found in the “Cellulolytic Composition present and/or added In Saccharification and/or Fermentation”-section below. The cellulolytic composition is present and/or added together with a glucoamylase, such as one disclosed in the “Glucoamylase Present And/Or Added in Saccharification and/or Fermentation”-section below.

### Fermentation

Fermentation is carried out in a fermentation medium. The fermentation medium includes the fermentation substrate, that is, the carbohydrate source that is metabolized by the fermenting organism. According to the invention the fermentation medium may comprise nutrients and growth stimulator(s) for the fermenting organism(s). Nutrient and growth stimulators are widely used in the art of fermentation and include nitrogen sources, such as ammonia; urea, vitamins and minerals, or combinations thereof.

As mentioned above the acid is added before exponential growth. After the fermenting organism is inoculated into the fermentation medium it passes through a number of phases. The initial phase is referred to as the “lag phase” and is a period of adaptation where no significant amount of fermentation product is produced. During the next two phases referred to as the “exponential growth phase” with increased growth and the “stationary phase”, which is the phase after maximum growth, significant amounts of fermentation product are produced. Fermentation cycles typically can go on for up to 96 hours or more.

### Fermenting Organisms

The term “Fermenting organism” refers to any organism, including bacterial and fungal organisms, especially yeast, suitable for use in a fermentation process and capable of producing the desired fermentation product. Especially suitable fermenting organisms are able to ferment, i.e., convert, sugars, such as glucose or maltose, directly or indirectly into the desired fermentation product, such as ethanol. Examples of fermenting organisms include fungal organisms, such as yeast. Preferred yeast includes strains of Saccharomyces spp., in particular, Saccharomyces cerevisiae.

Suitable concentrations of the viable fermenting organism during fermentation, such as SSF, are well known in the art or can easily be determined by the skilled person in the art. In one embodiment the fermenting organism, such as ethanol fermenting yeast, (e.g., Saccharomyces cerevisiae) is added to the fermentation medium so that the viable fermenting organism, such as yeast, count per mL of fermentation medium is in the range from 10⁵ to 10¹², preferably from 10⁷ to 10¹⁰, especially about 5×10⁷.

Examples of commercially available yeast includes, e.g., RED START™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties).

### Fermentation Products

The term “fermentation product” means a product produced by a process including a fermentation step using a fermenting organism. Fermentation products contemplated according to the invention include alcohols (e.g., ethanol, methanol, butanol; polyols such as glycerol, sorbitol and inositol); organic acids (e.g., citric acid, acetic acid, itaconic acid, lactic acid, succinic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones. In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer. Preferably processes of the invention are used for producing an alcohol, such as ethanol. The fermentation product, such as ethanol, obtained according to the invention, may be used as fuel, which is typically blended with gasoline. However, in the case of ethanol it may also be used as potable ethanol.

### Recovery

Subsequent to fermentation, or SSF, the fermentation product (i.e., ethanol) may be separated from the fermentation medium. The slurry may be distilled to extract the desired fermentation product (i.e., ethanol). Alternatively the desired fermentation product (i.e., ethanol) may be extracted from the fermentation medium by micro or membrane filtration techniques. The fermentation product (i.e., ethanol) may also be recovered by stripping or other method well known in the art.

Alpha-Amylase Present and/or Added in Liquefaction

According to the invention an alpha-amylase is present and/or added in liquefaction optionally together with a protease, glucoamylase, and/or optional pullulanase.

The alpha-amylase added in liquefaction step i) may be any alpha-amylase. Preferred are bacterial alpha-amylases, which typically are stable at temperature, used during liquefaction.

### Bacterial Alpha-Amylase

The term “bacterial alpha-amylase” means any bacterial alpha-amylase classified under EC 3.2.1.1. A bacterial alpha-amylase used according to the invention may, e.g., be derived from a strain of the genus Bacillus, which is sometimes also referred to as the genus Geobacillus. In an embodiment the Bacillus alpha-amylase is derived from a strain of Bacillus amyloliquefaciens, Bacillus licheniformis, Bacillus stearothermophilus, or Bacillus subtilis, but may also be derived from other Bacillus sp.

Specific examples of bacterial alpha-amylases include the Bacillus stearothermophilus alpha-amylase of SEQ ID NO: 3 in WO 99/19467, the Bacillus amyloliquefaciens alpha-amylase of SEQ ID NO: 5 in WO 99/19467, and the Bacillus licheniformis alpha-amylase of SEQ ID NO: 4 in WO 99/19467 or SEQ ID NO: 21 herein (all sequences are hereby incorporated by reference). In an embodiment the alpha-amylase may be an enzyme having a degree of identity of at least 60%, e.g., at least 70%, at least 80%, at least 90%, at least 95%, at least 96%, at least 97%, at least 98% or at least 99% to any of the sequences shown in SEQ ID NOS: 3, 4 or 5, respectively, in WO 99/19467.

In an embodiment the alpha-amylase may be an enzyme having a degree of identity of at least 60%, e.g., at least 70%, at least 80%, at least 90%, at least 91%, at least 92%, at least 93%, at least 94%, at least 95%, at least 96%, at least 97%, at least 98% or at least 99% to any of the sequences shown in SEQ ID NO: 3 in WO 99/19467 or SEQ ID NO: 1 herein.

In a preferred embodiment the alpha-amylase is derived from Bacillus stearothermophilus. The Bacillus stearothermophilus alpha-amylase may be a mature wild-type or a mature variant thereof. The mature Bacillus stearothermophilus alpha-amylases may naturally be truncated during recombinant production. For instance, the Bacillus stearothermophilus alpha-amylase may be a truncated so it has around 491 amino acids (compared to SEQ ID NO: 3 in WO 99/19467) or SEQ ID NO: 1 herein.

The Bacillus alpha-amylase may also be a variant and/or hybrid. Examples of such a variant can be found in any of WO 96/23873, WO 96/23874, WO 97/41213, WO 99/19467, WO 00/60059, and WO 02/10355 (all documents are hereby incorporated by reference). Specific alpha-amylase variants are disclosed in U.S. Pat. Nos. 6,093,562, 6,187,576, 6,297,038, and 7,713,723 (hereby incorporated by reference) and include Bacillus stearothermophilus alpha-amylase (often referred to as BSG alpha-amylase) variants having a deletion of one or two amino acids at positions R179, G180, 1181 and/or G182, preferably a double deletion disclosed in WO 96/23873—see, e.g., page 20, lines 1-10 (hereby incorporated by reference), preferably corresponding to deletion of positions I181 and G182 compared to the amino acid sequence of Bacillus stearothermophilus alpha-amylase set forth in SEQ ID NO: 3 disclosed in WO 99/19467 or SEQ ID NO: 1 herein or the deletion of amino acids R179 and G180 using SEQ ID NO: 3 in WO 99/19467 or SEQ ID NO: 1 herein for numbering (which reference is hereby incorporated by reference). Even more preferred are Bacillus alpha-amylases, especially Bacillus stearothermophilus alpha-amylases, which have a double deletion corresponding to a deletion of positions 181 and 182 and further optionally comprise a N193F substitution (also denoted I181*+G182*+N193F) compared to the wild-type BSG alpha-amylase amino acid sequence set forth in SEQ ID NO: 3 disclosed in WO 99/19467 or SEQ ID NO: 1 herein. The bacterial alpha-amylase may also have a substitution in a position corresponding to S239, in particular S239Q, in the Bacillus licheniformis alpha-amylase shown in SEQ ID NO: 4 in WO 99/19467 or SEQ ID NO: 21 herein, or a S242, in particular S242Q, and/or E188P variant of the Bacillus stearothermophilus alpha-amylase of SEQ ID NO: 3 in WO 99/19467 or SEQ ID NO: 1 herein.

In an embodiment the variant is a S242A, E or Q variant, preferably a S242Q variant, of the Bacillus stearothermophilus alpha-amylase (using SEQ ID NO: 1 herein for numbering).

In an embodiment the variant is a position E188 variant, preferably E188P variant of the Bacillus stearothermophilus alpha-amylase (using SEQ ID NO: 1 herein for numbering).

The bacterial alpha-amylase may in an embodiment be a truncated Bacillus licheniformis alpha-amylase. Especially the truncation is so that the Bacillus stearothermophilus alpha-amylase shown in SEQ ID NO: 3 in WO 99/19467 or SEQ ID NO: 1 herein, is around 491 amino acids long, such as from 480 to 495 amino acids long.

### Bacterial Hybrid Alpha-Amylases

The bacterial alpha-amylase may also be a hybrid bacterial alpha-amylase, e.g., an alpha-amylase comprising 445 C-terminal amino acid residues of the Bacillus licheniformis alpha-amylase (shown in SEQ ID NO: 4 of WO 99/19467) and the 37 N-terminal amino acid residues of the alpha-amylase derived from Bacillus amyloliquefaciens (shown in SEQ ID NO: 5 of WO 99/19467). In a preferred embodiment this hybrid has one or more, especially all, of the following substitutions:

G48A+T49I+G107A+H156Y+A181T+N190F+I201F+A209V+Q264S (using the Bacillus licheniformis numbering in SEQ ID NO: 4 of WO 99/19467) or SEQ ID NO: 21 herein. Also preferred are variants having one or more of the following mutations (or corresponding mutations in other Bacillus alpha-amylases): H154Y, A181T, N190F, A209V and Q264S and/or the deletion of two residues between positions 176 and 179, preferably the deletion of E178 and G179 (using SEQ ID NO: 5 of WO 99/19467 for position numbering).

In an embodiment the bacterial alpha-amylase is the mature part of the chimeric alpha-amylase disclosed in Richardson et al. (2002), The Journal of Biological Chemistry, Vol. 277, No 29, Issue 19 July, pp. 267501-26507, referred to as BD5088 or a variant thereof. This alpha-amylase is the same as the one shown in SEQ ID NO: 2 in WO 2007134207. The mature enzyme sequence starts after the initial “Met” amino acid in position 1.

### Thermostable Alpha-Amylase

According to the invention the alpha-amylase may be a thermostable alpha-amylase, such as a thermostable bacterial alpha-amylase, preferably from Bacillus stearothermophilus. In an embodiment the alpha-amylase used according to the invention has a T½ (min) at pH 4.5, 85° C., 0.12 mM CaCl₂ of at least 10 determined as described in Example 1.

In an embodiment the thermostable alpha-amylase has a T½ (min) at pH 4.5, 85° C., 0.12 mM CaCl₂, of at least 15.

In an embodiment the thermostable alpha-amylase has a T½ (min) at pH 4.5, 85° C., 0.12 mM CaCl₂, of as at least 20.

In an embodiment the thermostable alpha-amylase has a T½ (min) at pH 4.5, 85° C., 0.12 mM CaCl₂, of as at least 25.

In an embodiment the thermostable alpha-amylase has a T½ (min) at pH 4.5, 85° C., 0.12 mM CaCl₂, of as at least 30.

In an embodiment the thermostable alpha-amylase has a T½ (min) at pH 4.5, 85° C., 0.12 mM CaCl₂, of as at least 40.

In an embodiment the thermostable alpha-amylase has a T½ (min) at pH 4.5, 85° C., 0.12 mM CaCl₂, of at least 50.

In an embodiment the thermostable alpha-amylase has a T½ (min) at pH 4.5, 85° C., 0.12 mM CaCl₂, of at least 60.

In an embodiment the thermostable alpha-amylase has a T½ (min) at pH 4.5, 85° C., 0.12 mM CaCl₂, between 10-70.

In an embodiment the thermostable alpha-amylase has a T½ (min) at pH 4.5, 85° C., 0.12 mM CaCl₂, between 15-70.

In an embodiment the thermostable alpha-amylase has a T½ (min) at pH 4.5, 85° C., 0.12 mM CaCl₂, between 20-70.

In an embodiment the thermostable alpha-amylase has a T½ (min) at pH 4.5, 85° C., 0.12 mM CaCl₂, between 25-70.

In an embodiment the thermostable alpha-amylase has a T½ (min) at pH 4.5, 85° C., 0.12 mM CaCl₂, between 30-70.

In an embodiment the thermostable alpha-amylase has a T½ (min) at pH 4.5, 85° C., 0.12 mM CaCl₂, between 40-70.

In an embodiment the thermostable alpha-amylase has a T½ (min) at pH 4.5, 85° C., 0.12 mM CaCl₂, between 50-70.

In an embodiment the thermostable alpha-amylase has a T½ (min) at pH 4.5, 85° C., 0.12 mM CaCl₂, between 60-70.

In an embodiment of the invention the alpha-amylase is an bacterial alpha-amylase, preferably derived from the genus Bacillus, especially a strain of Bacillus stearothermophilus, in particular the Bacillus stearothermophilus as disclosed in WO 99/019467 as SEQ ID NO: 3 (SEQ ID NO: 1 herein) with one or two amino acids deleted at positions R179, G180, I181 and/or G182, in particular with R179 and G180 deleted, or with I181 and G182 deleted, with mutations in below list of mutations.

In preferred embodiments the Bacillus stearothermophilus alpha-amylases have double deletion I181+G182, and optional substitution N193F, further comprising mutations selected from below list:

V59A+Q89R+G112D+E129V+K177L+R179E+K220P+N224L+Q254S;

V59A+Q89R+E129V+K177L+R179E+H208Y+K220P+N224L+Q254S;

V59A+Q89R+E129V+K177L+R179E+K220P+N224L+Q254S+D269E+D281N;

V59A+Q89R+E129V+K177L+R179E+K220P+N224L+Q254S+I270L;

V59A+Q89R+E129V+K177L+R179E+K220P+N224L+Q254S+H274K;

V59A+Q89R+E129V+K177L+R179E+K220P+N224L+Q254S+Y276F;

V59A+E129V+R157Y+K177L+R179E+K220P+N224L+S242Q+Q254S;

V59A+E129V+K177L+R179E+H208Y+K220P+N224L+S242Q+Q254S;

59A+E129V+K177L+R179E+K220P+N224L+S242Q+Q254S;

V59A+E129V+K177L+R179E+K220P+N224L+S242Q+Q254S+H274K;

V59A+E129V+K177L+R179E+K220P+N224L+S242Q+Q254S+Y276F;

V59A+E129V+K177L+R179E+K220P+N224L+S242Q+Q254S+D281N;

V59A+E129V+K177L+R179E+K220P+N224L+S242Q+Q254S+M284T;

V59A+E129V+K177L+R179E+K220P+N224L+S242Q+Q254S+G416V;

V59A+E129V+K177L+R179E+K220P+N224L+Q254S;

V59A+E129V+K177L+R179E+K220P+N224L+Q254S+M284T;

A91 L+M961+E129V+K177L+R179E+K220P+N224L+S242Q+Q254S;

E129V+K177L+R179E;

E129V+K177L+R179E+K220P+N224L+S242Q+Q254S;

E129V+K177L+R179E+K220P+N224L+S242Q+Q254S+Y276F+L427M;

E129V+K177L+R179E+K220P+N224L+S242Q+Q254S+M284T;

E129V+K177L+R179E+K220P+N224L+S242Q+Q254S+N376*+1377*;

E129V+K177L+R179E+K220P+N224L+Q254S;

E129V+K177L+R179E+K220P+N224L+Q254S+M284T;

E129V+K177L+R179E+S242Q;

E129V+K177L+R179V+K220P+N224L+S242Q+Q254S;

K220P+N224L+S242Q+Q254S;

M284V;

V59A+Q89R+E129V+K177L+R179E+Q254S+M284V.

V59A+E129V+K177L+R179E+Q254S+M284V;

In a preferred embodiment the alpha-amylase is selected from the group of Bacillus stearothermophilus alpha-amylase variants with deletion I181*+G182*, and optionally substitution N193F, and additionally one of the following set of substitutions

E129V+K177L+R179E;

V59A+Q89R+E129V+K177L+R179E+H208Y+K220P+N224L+Q254S;

V59A+Q89R+E129V+K177L+R179E+Q254S+M284V;

V59A+E129V+K177L+R179E+Q254S+M284V; and

N193F+E129V+K177L+R179E+K220P+N224L+S242Q+Q254S (using SEQ ID NO: 1 herein for numbering).

It should be understood that when referring to Bacillus stearothermophilus alpha-amylase and variants thereof they are normally produced in truncated form. In particular, the truncation may be so that the Bacillus stearothermophilus alpha-amylase shown in SEQ ID NO: 3 in WO 99/19467 or SEQ ID NO: 1 herein, or variants thereof, are truncated in the C-terminal and are typically around 491 amino acids long, such as from 480-495 amino acids long.

In a preferred embodiment the alpha-amylase variant may be an enzyme having a degree of identity of at least 60%, e.g., at least 70%, at least 80%, at least 90%, at least 95%, at least 91%, at least 92%, at least 93%, at least 94%, at least 95%, at least 96%, at least 97%, at least 98% or at least 99%, but less than 100% to the sequence shown in SEQ ID NO: 3 in WO 99/19467 or SEQ ID NO: 1 herein.

In an embodiment the bacterial alpha-amylase, e.g., Bacillus alpha-amylase, such as especially Bacillus stearothermophilus alpha-amylase, or variant thereof, is dosed to liquefaction in a concentration between 0.01-10 KNU-Ng DS, e.g., between 0.02 and 5 KNU-Ng DS, such as 0.03 and 3 KNU-A, preferably 0.04 and 2 KNU-Ng DS, such as especially 0.01 and 2 KNU-A/g DS. In an embodiment the bacterial alpha-amylase, e.g., Bacillus alpha-amylase, such as especially Bacillus stearothermophilus alpha-amylases, or variant thereof, is dosed to liquefaction in a concentration of between 0.0001-1 mg EP(Enzyme Protein)/g DS, e.g., 0.0005-0.5 mg EP/g DS, such as 0.001-0.1 mg EP/g DS.

Protease Present and/or Added in Liquefaction

According to the invention a protease is optionally present and/or added in liquefaction together with the alpha-amylase, and an optional glucoamylase, and/or pullulanase.

Proteases are classified on the basis of their catalytic mechanism into the following groups: Serine proteases (S), Cysteine proteases (C), Aspartic proteases (A), Metallo proteases (M), and Unknown, or as yet unclassified, proteases (U), see Handbook of Proteolytic Enzymes, A. J. Barrett, N. D. Rawlings, J. F. Woessner (eds), Academic Press (1998), in particular the general introduction part.

In a preferred embodiment the thermostable protease used according to the invention is a “metallo protease” defined as a protease belonging to EC 3.4.24 (metalloendopeptidases); preferably EC 3.4.24.39 (acid metallo proteinases).

To determine whether a given protease is a metallo protease or not, reference is made to the above “Handbook of Proteolytic Enzymes” and the principles indicated therein. Such determination can be carried out for all types of proteases, be it naturally occurring or wild-type proteases; or genetically engineered or synthetic proteases.

Protease activity can be measured using any suitable assay, in which a substrate is employed, that includes peptide bonds relevant for the specificity of the protease in question. Assay-pH and assay-temperature are likewise to be adapted to the protease in question. Examples of assay-pH-values are pH 6, 7, 8, 9, 10, or 11. Examples of assay-temperatures are 30, 35, 37, 40, 45, 50, 55, 60, 65, 70 or 80° C.

Examples of protease substrates are casein, such as Azurine-Crosslinked Casein (AZCL-casein). Two protease assays are described below in the “Materials & Methods”-section, of which the so-called “AZCL-Casein Assay” is the preferred assay.

In an embodiment the thermostable protease has at least 20%, such as at least 30%, such as at least 40%, such as at least 50%, such as at least 60%, such as at least 70%, such as at least 80%, such as at least 90%, such as at least 95%, such as at least 100% of the protease activity of the Protease 196 variant or Protease Pfu determined by the AZCL-casein assay described in the “Materials & Methods” section.

There are no limitations on the origin of the protease used in a process of the invention as long as it fulfills the thermostability properties defined below.

In one embodiment the protease is of fungal origin.

The protease may be a variant of, e.g., a wild-type protease as long as the protease has the thermostability properties defined herein. In a preferred embodiment the thermostable protease is a variant of a metallo protease as defined above. In an embodiment the thermostable protease used in a process of the invention is of fungal origin, such as a fungal metallo protease, such as a fungal metallo protease derived from a strain of the genus Thermoascus, preferably a strain of Thermoascus aurantiacus, especially Thermoascus aurantiacus CGMCC No. 0670 (classified as EC 3.4.24.39).

In an embodiment the thermostable protease is a variant of the mature part of the metallo protease shown in SEQ ID NO: 2 disclosed in WO 2003/048353 or the mature part of SEQ ID NO: 1 in WO 2010/008841 and shown as SEQ ID NO: 3 herein further with mutations selected from below list:

S5*+D79L+S87P+A112P+D142L;

D79L+S87P+A112P+T124V+D142L;

S5*+N26R+D79L+S87P+A112P+D142L;

N26R+T46R+D79L+S87P+A112P+D142L;

T46R+D79L+S87P+T116V+D142L;

D79L+P81R+S87P+A112P+D142L;

A27K+D79L+S87P+A112P+T124V+D142L;

D79L+Y82F+S87P+A112P+T124V+D142L;

D79L+Y82F+S87P+A112P+T124V+D142L;

D79L+S87P+A112P+T124V+A126V+D142L;

D79L+S87P+A112P+D142L;

D79L+Y82F+S87P+A112P+D142L;

S38T+D79L+S87P+A112P+A126V+D142L;

D79L+Y82F+S87P+A112P+A126V+D142L;

A27K+D79L+S87P+A112P+A126V+D142L;

D79L+S87P+N98C+A112P+G135C+D142L;

D79L+S87P+A112P+D142L+T141C+M161C;

S36P+D79L+S87P+A112P+D142L;

A37P+D79L+S87P+A112P+D142L;

S49P+D79L+S87P+A112P+D142L;

S50P+D79L+S87P+A112P+D142L;

D79L+S87P+D104P+A112P+D142L;

D79L+Y82F+S87G+A112P+D142L;

S70V+D79L+Y82F+S87G+Y97W+A112P+D142L;

D79L+Y82F+S87G+Y97W+D104P+A112P+D142L;

S70V+D79L+Y82F+S87G+A112P+D142L;

D79L+Y82F+S87G+D104P+A112P+D142L;

D79L+Y82F+S87G+A112P+A126V+D142L;

Y82F+S87G+S70V+D79L+D104P+A112P+D142L;

Y82F+S87G+D79L+D104P+A112P+A126V+D142L;

A27K+D79L+Y82F+S87G+D104P+A112P+A126V+D142L;

A27K+Y82F+S87G+D104P+A112P+A126V+D142L;

A27K+D79L+Y82F+D104P+A112P+A126V+D142L;

A27K+Y82F+D104P+A112P+A126V+D142L;

A27K+D79L+S87P+A112P+D142L;

D79L+S87P+D142L.

In an preferred embodiment the thermostable protease is a variant of the metallo protease disclosed as the mature part of SEQ ID NO: 2 disclosed in WO 2003/048353 or the mature part of SEQ ID NO: 1 in WO 2010/008841 or SEQ ID NO: 3 herein with the following mutations:

### D79L+S87P+A112P+D142L;

### D79L+S87P+D142L; or

### A27K+D79L+Y82F+S87G+D104P+A112P+A126V+D142L.

In an embodiment the protease variant has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the mature part of the polypeptide of SEQ ID NO: 2 disclosed in WO 2003/048353 or the mature part of SEQ ID NO: 1 in WO 2010/008841 or SEQ ID NO: 3 herein.

The thermostable protease may also be derived from any bacterium as long as the protease has the thermostability properties defined according to the invention.

In an embodiment the thermostable protease is derived from a strain of the bacterium Pyrococcus, such as a strain of Pyrococcus furiosus (pfu protease)

In an embodiment the protease is one shown as SEQ ID NO: 1 in U.S. Pat. No. 6,358,726-B1 (Takara Shuzo Company), or SEQ ID NO: 13 herein.

In another embodiment the thermostable protease is one disclosed in SEQ ID NO: 13 herein or a protease having at least 80% identity, such as at least 85%, such as at least 90%, such as at least 95%, such as at least 96%, such as at least 97%, such as at least 98%, such as at least 99% identity to SEQ ID NO: 1 in U.S. Pat. No. 6,358,726-B1 or SEQ ID NO: 13 herein. The Pyroccus furiosus protease can be purchased from Takara Bio, Japan.

The Pyrococcus furiosus protease is a thermostable protease according to the invention. The commercial product Pyrococcus furiosus protease (Pfu S) was found to have a thermostability of 110% (80° C./70° C.) and 103% (90° C./70° C.) at pH 4.5 determined as described in Example 2.

In one embodiment a thermostable protease used in a process of the invention has a thermostability value of more than 20% determined as Relative Activity at 80° C./70° C. determined as described in Example 2.

In an embodiment the protease has a thermostability of more than 30%, more than 40%, more than 50%, more than 60%, more than 70%, more than 80%, more than 90%, more than 100%, such as more than 105%, such as more than 110%, such as more than 115%, such as more than 120% determined as Relative Activity at 80° C./70° C.

In an embodiment protease has a thermostability of between 20 and 50%, such as between 20 and 40%, such as 20 and 30% determined as Relative Activity at 80° 0170° C. In an embodiment the protease has a thermostability between 50 and 115%, such as between 50 and 70%, such as between 50 and 60%, such as between 100 and 120%, such as between 105 and 115% determined as Relative Activity at 80° C./70° C.

In an embodiment the protease has a thermostability value of more than 10% determined as Relative Activity at 85° C./70° C. determined as described in Example 2.

In an embodiment the protease has a thermostability of more than 10%, such as more than 12%, more than 14%, more than 16%, more than 18%, more than 20%, more than 30%, more than 40%, more that 50%, more than 60%, more than 70%, more than 80%, more than 90%, more than 100%, more than 110% determined as Relative Activity at 85° C./70° C.

In an embodiment the protease has a thermostability of between 10 and 50%, such as between 10 and 30%, such as between 10 and 25% determined as Relative Activity at 85° C./70° C.

In an embodiment the protease has more than 20%, more than 30%, more than 40%, more than 50%, more than 60%, more than 70%, more than 80%, more than 90% determined as Remaining Activity at 80° C.; and/or

In an embodiment the protease has more than 20%, more than 30%, more than 40%, more than 50%, more than 60%, more than 70%, more than 80%, more than 90% determined as Remaining Activity at 84° C.

Determination of “Relative Activity” and “Remaining Activity” is done as described in Example 2.

In an embodiment the protease may have a themostability for above 90, such as above 100 at 85° C. as determined using the Zein-BCA assay as disclosed in Example 3.

In an embodiment the protease has a themostability above 60%, such as above 90%, such as above 100%, such as above 110% at 85° C. as determined using the Zein-BCA assay.

In an embodiment protease has a themostability between 60-120, such as between 70-120%, such as between 80-120%, such as between 90-120%, such as between 100-120%, such as 110-120% at 85° C. as determined using the Zein-BCA assay.

In an embodiment the thermostable protease has at least 20%, such as at least 30%, such as at least 40%, such as at least 50%, such as at least 60%, such as at least 70%, such as at least 80%, such as at least 90%, such as at least 95%, such as at least 100% of the activity of the JTP196 protease variant or Protease Pfu determined by the AZCL-casein assay.

Glucoamylase Present and/or Added in Liquefaction

According to the invention a glucoamylase may optionally be present and/or added in liquefaction step i). In a preferred embodiment the glucoamylase is added together with or separately from the alpha-amylase and/or the protease and/or pullulanase.

In an embodiment the glucoamylase has a Relative Activity heat stability at 85° C. of at least 20%, at least 30%, preferably at least 35% determined as described in Example 4 (heat stability).

In an embodiment the glucoamylase has a relative activity pH optimum at pH 5.0 of at least 90%, preferably at least 95%, preferably at least 97%, such as 100% determined as described in Example 4 (pH optimum).

In an embodiment the glucoamylase has a pH stability at pH 5.0 of at least at least 80%, at least 85%, at least 90% determined as described in Example 4 (pH stability).

In an embodiment the glucoamylase, such as a Penicillium oxalicum glucoamylase variant, used in liquefaction has a thermostability determined as DSC Td at pH 4.0 as described in Example 15 of at least 70° C., preferably at least 75° C., such as at least 80° C., such as at least 81° C., such as at least 82° C., such as at least 83° C., such as at least 84° C., such as at least 85° C., such as at least 86° C., such as at least 87%, such as at least 88° C., such as at least 89° C., such as at least 90° C. In an embodiment the glucoamylase, such as a Penicillium oxalicum glucoamylase variant has a thermostability determined as DSC Td at pH 4.0 as described in Example 15 in the range between 70° C. and 95° C., such as between 80° C. and 90° C.

In an embodiment the glucoamylase, such as a Penicillium oxalicum glucoamylase variant, used in liquefaction has a thermostability determined as DSC Td at pH 4.8 as described in Example 15 of at least 70° C., preferably at least 75° C., such as at least 80° C., such as at least 81° C., such as at least 82° C., such as at least 83° C., such as at least 84° C., such as at least 85° C., such as at least 86° C., such as at least 87%, such as at least 88° C., such as at least 89° C., such as at least 90° C., such as at least 91° C. In an embodiment the glucoamylase, such as a Penicillium oxalicum glucoamylase variant has a thermostability determined as DSC Td at pH 4.8 as described in Example 15 in the range between 70° C. and 95° C., such as between 80° C. and 90° C.

In an embodiment the glucoamylase, such as a Penicillium oxalicum glucoamylase variant, used in liquefaction has a residual activity determined as described in Example 16 of at least 100% such as at least 105%, such as at least 110%, such as at least 115%, such as at least 120%, such as at least 125%. In an embodiment the glucoamylase, such as a Penicillium oxalicum glucoamylase variant has a thermostability determined as residual activity as described in Example 16 in the range between 100% and 130%.

In a specific and preferred embodiment the glucoamylase, preferably of fungal origin, preferably a filamentous fungi, is from a strain of the genus Penicillium, especially a strain of Penicillium oxalicum, in particular the Penicillium oxalicum glucoamylase disclosed as SEQ ID NO: 2 in WO 2011/127802 (which is hereby incorporated by reference) and shown in SEQ ID NO: 9 or 14 herein.

In an embodiment the glucoamylase has at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99% or 100% identity to the mature polypeptide shown in SEQ ID NO: 2 in WO 2011/127802 or SEQ ID NOs: 9 or 14 herein.

In a preferred embodiment the glucoamylase is a variant of the Penicillium oxalicum glucoamylase disclosed as SEQ ID NO: 2 in WO 2011/127802 and shown in SEQ ID NO: 9 and 14 herein, having a K79V substitution (using the mature sequence shown in SEQ ID NO: 14 herein for numbering). The K79V glucoamylase variant has reduced sensitivity to protease degradation relative to the parent as disclosed in WO 2013/036526 (which are hereby incorporated by reference).

In an embodiment the glucoamylase is derived from Penicillium oxalicum.

In an embodiment the glucoamylase is a variant of the Penicillium oxalicum glucoamylase disclosed as SEQ ID NO: 2 in WO 2011/127802 and shown in SEQ ID NO: 9 and 14 herein. In a preferred embodiment the Penicillium oxalicum glucoamylase is the one disclosed as SEQ ID NO: 2 in WO 2011/127802 and shown in SEQ ID NO: 9 and 14 herein having Val (V) in position 79 (using SEQ ID NO: 14 herein for numbering).

Contemplated Penicillium oxalicum glucoamylase variants are disclosed in WO 2013/053801 which is hereby incorporated by reference.

In an embodiment these variants have reduced sensitivity to protease degradation.

In an embodiment these variant have improved thermostability compared to the parent.

More specifically, in an embodiment the glucoamylase has a K79V substitution (using SEQ ID NO: 14 herein for numbering), corresponding to the PE001 variant, and further comprises at least one of the following substitutions or combination of substitutions:

### T65A; or

### Q327F; or

### E501V; or

### Y504T; or

### Y504*; or

### T65A+Q327F; or

### T65A+E501V; or

### T65A+Y504T; or

### T65A+Y504*; or

### Q327F+E501V; or

### Q327F+Y504T; or

### Q327F+Y504*; or

### E501V+Y504T; or

### E501V+Y504*; or

### T65A+Q327F+E501V; or

### T65A+Q327F+Y504T; or

### T65A+E501V+Y504T; or

### Q327F+E501V+Y504T; or

### T65A+Q327F+Y504*; or

### T65A+E501V+Y504*; or

### Q327F+E501V+Y504*; or

### T65A+Q327F+E501V+Y504T; or

### T65A+Q327F+E501V+Y504*;

### E501V+Y504T; or

### T65A+K161S; or

### T65A+Q405T; or

### T65A+Q327W; or

### T65A+Q327F; or

### T65A+Q327Y; or

### P11F+T65A+Q327F; or

### R1K+D3W+K5Q+G7V+N8S+T10K+P11S+T65A+Q327F; or

### P2N+P4S+P11F+T65A+Q327F; or

### P11F+D26C+K33C+T65A+Q327F; or

### P2N+P4S+P11F+T65A+Q327W+E501V+Y504T; or

### R1E+D3N+P4G+G6R+G7A+N8A+T10D+P11D+T65A+Q327F; or

### P11F+T65A+Q327W; or

### P2N+P4S+P11F+T65A+Q327F+E501V+Y504T; or

### P11F+T65A+Q327W+E501V+Y504T; or

### T65A+Q327F+E501V+Y504T; or

### T65A+S105P+Q327W; or

### T65A+S105P+Q327F; or

### T65A+Q327W+S364P; or

### T65A+Q327F+S364P; or

### T65A+S103N+Q327F; or

### P2N+P4S+P11F+K34Y+T65A+Q327F; or

### P2N+P4S+P11F+T65A+Q327F+D445N+V4475; or

### P2N+P4S+P11F+T65A+I172V+Q327F; or

### P2N+P4S+P11F+T65A+Q327F+N502*; or

### P2N+P4S+P11F+T65A+Q327F+N502T+P563S+K571E; or

### P2N+P4S+P11F+R31S+K33V+T65A+Q327F+N564D+K571S; or

### P2N+P4S+P11F+T65A+Q327F+S377T; or

### P2N+P4S+P11F+T65A+V325T+Q327W; or

### P2N+P4S+P11F+T65A+Q327F+D445N+V447S+E501V+Y504T; or

### P2N+P4S+P11F+T65A+I172V+Q327F+E501V+Y504T; or

### P2N+P4S+P11F+T65A+Q327F+S377T+E501V+Y504T; or

### P2N+P4S+P11F+D26N+K34Y+T65A+Q327F; or

### P2N+P4S+P11F+T65A+Q327F+I375A+E501V+Y504T; or

### P2N+P4S+P11F+T65A+K218A+K221D+Q327F+E501V+Y504T; or

### P2N+P4S+P11F+T65A+S103N+Q327F+E501V+Y504T; or

### P2N+P4S+T10D+T65A+Q327F+E501V+Y504T; or

### P2N+P4S+F12Y+T65A+Q327F+E501V+Y504T; or

### K5A+P11F+T65A+Q327F+E501V+Y504T; or

### P2N+P4S+T10E+E18N+T65A+Q327F+E501V+Y504T; or

### P2N+T10E+E18N+T65A+Q327F+E501V+Y504T; or

### P2N+P4S+P11F+T65A+Q327F+E501V+Y504T+T568N; or

### P2N+P4S+P11F+T65A+Q327F+E501V+Y504T+K524T+G526A; or

### P2N+P4S+P11F+K34Y+T65A+Q327F+D445N+V447S+E501V+Y504T; or

### P2N+P4S+P11F+R31S+K33V+T65A+Q327F+D445N+V447S+E501V+Y504T; or

### P2N+P4S+P11F+D26N+K34Y+T65A+Q327F+E501V+Y504T; or

### P2N+P4S+P11F+T65A+F80*+Q327F+E501V+Y504T; or

### P2N+P4S+P11F+T65A+K112S+Q327F+E501V+Y504T; or

### P2N+P4S+P11F+T65A+Q327F+E501V+Y504T+T516P+K524T+G526A; or

### P2N+P4S+P11F+T65A+Q327F+E501V+N502T+Y504*; or

### P2N+P4S+P11F+T65A+Q327F+E501V+Y504T; or

### P2N+P4S+P11F+T65A+S103N+Q327F+E501V+Y504T; or

### K5A+P11F+T65A+Q327F+E501V+Y504T; or

### P2N+P4S+P11F+T65A+Q327F+E501V+Y504T+T516P+K524T+G526A; or

### P2N+P4S+P11F+T65A+V79A+Q327F+E501V+Y504T; or

### P2N+P4S+P11F+T65A+V79G+Q327F+E501V+Y504T; or

### P2N+P4S+P11F+T65A+V791+Q327F+E501V+Y504T; or

### P2N+P4S+P11F+T65A+V79L+Q327F+E501V+Y504T; or

### P2N+P4S+P11F+T65A+V79S+Q327F+E501V+Y504T; or

### P2N+P4S+P11F+T65A+L72V+Q327F+E501V+Y504T; or

### S255N+Q327F+E501V+Y504T; or

### P2N+P4S+P11F+T65A+E74N+V79K+Q327F+E501V+Y504T; or

### P2N+P4S+P11F+T65A+G220N+Q327F+E501V+Y504T; or

### P2N+P4S+P11F+T65A+Y245N+Q327F+E501V+Y504T; or

### P2N+P4S+P11F+T65A+Q253N+Q327F+E501V+Y504T; or

### P2N+P4S+P11F+T65A+D279N+Q327F+E501V+Y504T; or

### P2N+P4S+P11F+T65A+Q327F+S359N+E501V+Y504T; or

### P2N+P4S+P11F+T65A+Q327F+D370N+E501V+Y504T; or

### P2N+P4S+P11F+T65A+Q327F+V460S+E501V+Y504T; or

### P2N+P4S+P11F+T65A+Q327F+V460T+P468T+E501V+Y504T; or

### P2N+P4S+P11F+T65A+Q327F+T463N+E501V+Y504T; or

### P2N+P4S+P11F+T65A+Q327F+S465N+E501V+Y504T; or P2N+P4S+P11F+T65A+Q327F+T477N+E501V+Y504T.

In a preferred embodiment the Penicillium oxalicum glucoamylase variant has a K79V substitution (using SEQ ID NO: 14 herein for numbering), corresponding to the PE001 variant, and further comprises one of the following mutations:

### P11F+T65A+Q327F; or

### P2N+P4S+P11F+T65A+Q327F; or

### P11F+D26C+K33C+T65A+Q327F; or

### P2N+P4S+P11F+T65A+Q327W+E501V+Y504T; or

### P2N+P4S+P11F+T65A+Q327F+E501V+Y504T; or

### P11F+T65A+Q327W+E501V+Y504T.

The glucoamylase may be added in amounts from 0.1-100 micrograms EP/g, such as 0.5-50 micrograms EP/g, such as 1-25 micrograms EP/g, such as 2-12 micrograms EP/g DS.

Pullulanase Present and/or Added in Liquefaction Step i)

Optionally a pullulanase may be present and/or added during liquefaction step i) together with an alpha-amylase, and/or protease and/or glucoamylase. As mentioned above a glucoamylase glucoamylase may also be present and/or added during liquefaction step i).

The pullulanase may be present and/or added in liquefaction step i) and/or saccharification step ii) or simultaneous saccharification and fermentation (SSF).

Pullulanases (E.C. 3.2.1.41, pullulan 6-glucano-hydrolase), are debranching enzymes characterized by their ability to hydrolyze the alpha-1,6-glycosidic bonds in, for example, amylopectin and pullulan.

Contemplated pullulanases according to the present invention include the pullulanases from Bacillus amyloderamificans disclosed in U.S. Pat. No. 4,560,651 (hereby incorporated by reference), the pullulanase disclosed as SEQ ID NO: 2 in WO 01/151620 (hereby incorporated by reference), the Bacillus deramificans disclosed as SEQ ID NO: 4 in WO 01/151620 (hereby incorporated by reference), and the pullulanase from Bacillus acidopullulyticus disclosed as SEQ ID NO: 6 in WO 01/151620 (hereby incorporated by reference) and also described in FEMS Mic. Let. (1994) 115, 97-106.

Additional pullulanases contemplated according to the present invention included the pullulanases from Pyrococcus woesei, specifically from Pyrococcus woesei DSM No. 3773 disclosed in WO92/02614.

In an embodiment the pullulanase is a family GH57 pullulanase. In an embodiment the pullulanase includes an X47 domain as disclosed in U.S. 61/289,040 published as WO 2011/087836 (which are hereby incorporated by reference). More specifically the pullulanase may be derived from a strain of the genus Thermococcus, including Thermococcus litoralis and Thermococcus hydrothermalis, such as the Thermococcus hydrothermalis pullulanase shown in SEQ ID NO: 11 truncated at site X4 right after the X47 domain (i.e., amino acids 1-782 in SEQ ID NOS: 11 and 12 herein). The pullulanase may also be a hybrid of the Thermococcus litoralis and Thermococcus hydrothermalis pullulanases or a T. hydrothermalis/T. litoralis hybrid enzyme with truncation site X4 disclosed in U.S. 61/289,040 published as WO 2011/087836 (which is hereby incorporated by reference) and disclosed in SEQ ID NO: 12 herein.

In another embodiment the pullulanase is one comprising an X46 domain disclosed in WO 2011/076123 (Novozymes).

The pullulanase may according to the invention be added in an effective amount which include the preferred amount of about 0.0001-10 mg enzyme protein per gram DS, preferably 0.0001-0.10 mg enzyme protein per gram DS, more preferably 0.0001-0.010 mg enzyme protein per gram DS. Pullulanase activity may be determined as NPUN. An Assay for determination of NPUN is described in the “Materials & Methods”-section below.

Suitable commercially available pullulanase products include PROMOZYME D, PROMOZYME™ D2 (Novozymes NS, Denmark), OPTIMAX L-300 (DuPont-Danisco, USA), and AMANO 8 (Amano, Japan).

Glucoamylase Present and/or Added in Saccharification and/or Fermentation

The glucoamylase present and/or added in saccharification, fermentation or simultaneous saccharification and fermentation (SSF) may be derived from any suitable source, e.g., derived from a microorganism or a plant. Preferred glucoamylases are of fungal or bacterial origin, selected from the group consisting of Aspergillus glucoamylases, in particular Aspergillus niger G1 or G2 glucoamylase (Boel et al. (1984), EMBO J. 3 (5), p. 1097-1102), or variants thereof, such as those disclosed in WO 92/00381, WO 00/04136 and WO 01/04273 (from Novozymes, Denmark); the A. awamori glucoamylase disclosed in WO 84/02921, Aspergillus oryzae glucoamylase (Agric. Biol. Chem. (1991), 55 (4), p. 941-949), or variants or fragments thereof. Other Aspergillus glucoamylase variants include variants with enhanced thermal stability: G137A and G139A (Chen et al. (1996), Prot. Eng. 9, 499-505); D257E and D293E/Q (Chen et al. (1995), Prot. Eng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Eng. 10, 1199-1204.

Other glucoamylases include Athelia rolfsii (previously denoted Corticium rolfsii) glucoamylase (see U.S. Pat. No. 4,727,026 and (Nagasaka et al. (1998) “Purification and properties of the raw-starch-degrading glucoamylases from Corticium rolfsii, Appl Microbiol Biotechnol 50:323-330), Talaromyces glucoamylases, in particular derived from Talaromyces emersonii (WO 99/28448), Talaromyces leycettanus (US patent no. Re. 32,153), Talaromyces duponti, Talaromyces thermophilus (U.S. Pat. No. 4,587,215). In a preferred embodiment the glucoamylase used during saccharification and/or fermentation is the Talaromyces emersonii glucoamylase disclosed in WO 99/28448.

Bacterial glucoamylases contemplated include glucoamylases from the genus Clostridium, in particular C. thermoamylolyticum (EP 135,138), and C. thermohydrosulfuricum (WO 86/01831).

Contemplated fungal glucoamylases include Trametes cingulate (SEQ ID NO: 20), Pachykytospora papyracea; and Leucopaxillus giganteus all disclosed in WO 2006/069289; or Peniophora rufomarginata disclosed in WO2007/124285; or a mixture thereof. Also hybrid glucoamylase are contemplated according to the invention. Examples include the hybrid glucoamylases disclosed in WO 2005/045018. Specific examples include the hybrid glucoamylase disclosed in Table 1 and 4 of Example 1 (which hybrids are hereby incorporated by reference).

In an embodiment the glucoamylase is derived from a strain of the genus Pycnoporus, in particular a strain of Pycnoporus as described in WO 2011/066576 (SEQ ID NOs 2, 4 or 6), such as SEQ ID NO: 18 herein, or from a strain of the genus Gloeophyllum, such as a strain of Gloeophyllum sepiarium or Gloeophyllum trabeum, in particular a strain of Gloeophyllum as described in WO 2011/068803 (SEQ ID NO: 2, 4, 6, 8, 10, 12, 14 or 16). In a preferred embodiment the glucoamylase is SEQ ID NO: 2 in WO 2011/068803 or SEQ ID NO: 15 herein.

In a preferred embodiment the glucoamylase is SEQ ID NO: 17 herein. In an embodiment the glucoamylase is derived from a strain of the genus Nigrofomes, in particular a strain of Nigrofomes sp. disclosed in WO 2012/064351 (SEQ ID NO: 2) (all references hereby incorporated by reference). Contemplated are also glucoamylases which exhibit a high identity to any of the above mentioned glucoamylases, i.e., at least 70%, at least 75%, at least 80%, at least 85%, at least 90%, at least 95%, at least 96%, at least 97%, at least 98%, at least 99% or even 100% identity to any one of the mature parts of the enzyme sequences mentioned above, such as any of SEQ ID NOs: 15, 17, 18 or 19 herein, preferably SEQ ID NO: 15 herein.

Glucoamylases may in an embodiment be added to the saccharification and/or fermentation in an amount of 0.0001-20 AGU/g DS, preferably 0.001-10 AGU/g DS, especially between 0.01-5 AGU/g DS, such as 0.1-2 AGU/g DS.

Glucoamylases may in an embodiment be added to the saccharification and/or fermentation in an amount of 1-1,000 μg EP/g DS, preferably 10-500 μg/gDS, especially between 25-250 μg/g DS.

In an embodiment the glucoamylase is added as a blend further comprising an alpha-amylase. In a preferred embodiment the alpha-amylase is a fungal alpha-amylase, especially an acid fungal alpha-amylase. The alpha-amylase is typically a side activity.

In an embodiment the glucoamylase is a blend comprising Talaromyces emersonii glucoamylase disclosed in WO 99/28448 as SEQ ID NO: 7 and Trametes cingulata glucoamylase disclosed as SEQ ID NO: 2 in WO 06/069289 and SEQ ID NO: 20 herein.

In an embodiment the glucoamylase is a blend comprising Talaromyces emersonii glucoamylase disclosed in WO 99/28448, Trametes cingulata glucoamylase disclosed as SEQ ID NO: 2 in WO 06/69289 and SEQ ID NO: 20 herein, and Rhizomucor pusillus alpha-amylase with Aspergillus niger glucoamylase linker and SBD disclosed as V039 in Table 5 in WO 2006/069290 or SEQ ID NO: 16 herein.

In an embodiment the glucoamylase is a blend comprising Talaromyces emersonii glucoamylase disclosed in WO99/28448, Trametes cingulata glucoamylase disclosed in WO 06/69289, and Rhizomucor pusillus alpha-amylase with Aspergillus niger glucoamylase linker and SBD disclosed as V039 in Table 5 in WO 2006/069290 or SEQ ID NO: 16 herein.

In an embodiment the glucoamylase is a blend comprising Gloeophyllum sepiarium glucoamylase shown as SEQ ID NO: 2 in WO 2011/068803 and Rhizomucor pusillus with an Aspergillus niger glucoamylase linker and starch-binding domain (SBD), disclosed SEQ ID NO: 3 in WO 2013/006756 with the following substitutions: G128D+D143N.

In an embodiment the alpha-amylase may be derived from a strain of the genus Rhizomucor, preferably a strain the Rhizomucor pusillus, such as the one shown in SEQ ID NO: 3 in WO2013/006756, or the genus Meripilus, preferably a strain of Meripilus giganteus. In a preferred embodiment the alpha-amylase is derived from a Rhizomucor pusillus with an Aspergillus niger glucoamylase linker and starch-binding domain (SBD), disclosed as V039 in Table 5 in WO 2006/069290 or SEQ ID NO: 16 herein.

In an embodiment the Rhizomucor pusillus alpha-amylase or the Rhizomucor pusillus alpha-amylase with an Aspergillus niger glucoamylase linker and starch-binding domain (SBD) has at least one of the following substitutions or combinations of substitutions: D165M; Y141W; Y141R; K136F; K192R; P224A; P224R; S123H+Y141W; G20S+Y141W; A76G+Y141W; G128D+Y141W; G128D+D143N; P219C+Y141W; N142D+D143N; Y141W+K192R; Y141W+D143N; Y141W+N383R; Y141W+P219C+A265C; Y141W+N142D+D143N; Y141W+K192R V410A; G128D+Y141W+D143N; Y141W+D143N+P219C; Y141W+D143N+K192R; G128D+D143N+K192R; Y141W+D143N+K192R+P219C; G128D+Y141W+D143N+K192R; or G128D+Y141W+D143N+K192R+P219C (using SEQ ID NO: 3 in WO 2013/006756 for numbering or SEQ ID NO: 16 herein).

In a preferred embodiment the glucoamylase blend comprises Gloeophyllum sepiarium glucoamylase (e.g., SEQ ID NO: 2 in WO 2011/068803 or SEQ ID NO: 15 herein) and Rhizomucor pusillus alpha-amylase.

In a preferred embodiment the glucoamylase blend comprises Gloeophyllum sepiarium glucoamylase shown as SEQ ID NO: 2 in WO 2011/068803 or SEQ ID NO: 15 herein and Rhizomucor pusillus with an Aspergillus niger glucoamylase linker and starch-binding domain (SBD), disclosed SEQ ID NO: 3 in WO 2013/006756 and SEQ ID NO: 16 herein with the following substitutions: G128D+D143N.

Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYME™ B4U, SPIRIZYME™ ULTRA, SPIRIZYME™ EXCEL, SPIRIZYME ACHIEVE™ and AMG™ E (from Novozymes NS); OPTIDEX™ 300, GC480, GC417 (from DuPont-Danisco); AMIGASE™ and AMIGASE™ PLUS (from DSM); G-ZYME™ G900, G-ZYME™ and G990 ZR (from DuPont-Danisco).

Cellulolytic Composition Present and/or Added in Saccharification and/or Fermentation

According to the invention a cellulolytic composition may be present in saccharification, fermentation or simultaneous saccharification and fermentation (SSF).

The cellulolytic composition comprises a beta-glucosidase, a cellobiohydrolase and an endoglucanase.

Examples of suitable cellulolytic composition can be found in WO 2008/151079 and WO 2013/028928 which are incorporated by reference.

In preferred embodiments the cellulolytic composition is derived from a strain of Trichoderma, Humicola, or Chrysosporium.

In an embodiment the cellulolytic composition is derived from a strain of Trichoderma reesei, Humicola insolens and/or Chrysosporium lucknowense.

In an embodiment the cellulolytic composition comprises a beta-glucosidase, preferably one derived from a strain of the genus Aspergillus, such as Aspergillus oryzae, such as the one disclosed in WO 2002/095014 or the fusion protein having beta-glucosidase activity disclosed in

WO 2008/057637, or Aspergillus fumigatus, such as one disclosed in WO 2005/047499 or an Aspergillus fumigatus beta-glucosidase variant disclosed in WO 2012/044915 (Novozymes), such as one with the following substitutions: F100D, S283G, N456E, F512Y; or a strain of the genus a strain Penicillium, such as a strain of the Penicillium brasilianum disclosed in WO 2007/019442, or a strain of the genus Trichoderma, such as a strain of Trichoderma reesei.

In an embodiment the cellulolytic composition comprises a GH61 polypeptide having cellulolytic enhancing activity such as one derived from the genus Thermoascus, such as a strain of Thermoascus aurantiacus, such as the one described in WO 2005/074656 as SEQ ID NO: 2; or one derived from the genus Thielavia, such as a strain of Thielavia terrestris, such as the one described in WO 2005/074647 as SEQ ID NO: 7 and SEQ ID NO: 8; or one derived from a strain of Aspergillus, such as a strain of Aspergillus fumigatus, such as the one described in WO 2010/138754 as SEQ ID NO: 1 and SEQ ID NO: 2; or one derived from a strain derived from Penicillium, such as a strain of Penicillium emersonii, such as the one disclosed in WO 2011/041397.

In an embodiment the cellulolytic composition comprises a cellobiohydrolase I (CBH I), such as one derived from a strain of the genus Aspergillus, such as a strain of Aspergillus fumigatus, such as the Cel7a CBH I disclosed in SEQ ID NO: 2 in WO 2011/057140, or a strain of the genus Trichoderma, such as a strain of Trichoderma reesei.

In an embodiment the cellulolytic composition comprises a cellobiohydrolase II (CBH II, such as one derived from a strain of the genus Aspergillus, such as a strain of Aspergillus fumigatus; or a strain of the genus Trichoderma, such as Trichoderma reesei, or a strain of the genus Thielavia, such as a strain of Thielavia terrestris, such as cellobiohydrolase II CEL6A from Thielavia terrestris.

In an embodiment the cellulolytic composition comprises a GH61 polypeptide having cellulolytic enhancing activity and a beta-glucosidase.

In an embodiment the cellulolytic composition comprises a GH61 polypeptide having cellulolytic enhancing activity, a beta-glucosidase, and a CBH I.

In an embodiment the cellulolytic composition comprises a GH61 polypeptide having cellulolytic enhancing activity, a beta-glucosidase, a CBH I, and a CBH II.

In an embodiment the cellulolytic composition is a Trichoderma reesei cellulolytic enzyme composition, further comprising Thermoascus aurantiacus GH61A polypeptide having cellulolytic enhancing activity (SEQ ID NO: 2 in WO 2005/074656), and Aspergillus oryzae beta-glucosidase fusion protein (WO 2008/057637).

In an embodiment the cellulolytic composition is a Trichoderma reesei cellulolytic enzyme composition, further comprising Thermoascus aurantiacus GH61A polypeptide having cellulolytic enhancing activity (SEQ ID NO: 2 in WO 2005/074656) and Aspergillus fumigatus beta-glucosidase (SEQ ID NO: 2 of WO 2005/047499).

In an embodiment the cellulolytic composition is a Trichoderma reesei cellulolytic enzyme composition further comprising Penicillium emersonii GH61A polypeptide having cellulolytic enhancing activity disclosed in WO 2011/041397 and Aspergillus fumigatus beta-glucosidase (SEQ ID NO: 2 of WO 2005/047499) or a variant thereof with one or more, such as all, of the following substitutions F100D, S283G, N456E, F512Y.

In a preferred embodiment the cellulolytic composition comprising one or more of the following components:

(i) an Aspergillus fumigatus cellobiohydrolase I;

(ii) an Aspergillus fumigatus cellobiohydrolase II;

(iii) an Aspergillus fumigatus beta-glucosidase or variant thereof; and

(iv) a Penicillium sp. GH61 polypeptide having cellulolytic enhancing activity; or homologs thereof.

In an preferred embodiment the cellulolytic composition is derived from Trichoderma reesei comprising GH61A polypeptide having cellulolytic enhancing activity derived from a strain of Penicillium emersonii (SEQ ID NO: 2 in WO 2011/041397, Aspergillus fumigatus beta-glucosidase (SEQ ID NO: 2 in WO 2005/047499) variant with the following substitutions: F100D, S283G, N456E, F512Y) disclosed in WO 2012/044915; Aspergillus fumigatus Cel7A CBH1 disclosed as SEQ ID NO: 6 in WO2011/057140 and Aspergillus fumigatus CBH II disclosed as SEQ ID NO: 18 in WO 2011/057140.

In an embodiment the cellulolytic composition is dosed from 0.0001-3 mg EP/g DS, preferably, 0.0005-2 mg EP/g DS, preferably 0.001-1 mg/g DS, more preferably 0.005-0.5 mg EP/g DS, and even more preferably 0.01-0.1 mg EP/g DS.

### Examples of Preferred Conventional Processes of the Invention

In a preferred embodiment the invention relates processes for producing fermentation products, such as especially ethanol, from starch-containing material comprising the steps of:

i) liquefying the starch-containing material at a temperature above the initial gelatinization temperature using an alpha-amylase derived from Bacillus stearothermophilus;
ii) saccharifying using a glucoamylase;
iii) fermenting using a fermenting organism;
wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.

In an embodiment the acid concentration is maintained between 10 and 100 mmoles/L fermentation medium.

In a preferred embodiment the invention relates processes for producing ethanol from starch-containing material comprising the steps of:

i) liquefying the starch-containing material at a temperature above the initial gelatinization temperature using an alpha-amylase derived from Bacillus stearothermophilus;
ii) saccharifying using a glucoamylase;
iii) fermenting using a fermenting organism;
wherein an acid selected from the group of acetic acid, benzoic acid, propionic acid, sorbic acid, formic acid, and succinic acid is present or added in fermentation so that the acid concentration in fermentation is maintained between above 0 (zero) and 100 mmoles/L fermentation medium and wherein the acid is added before the exponential growth phase of the fermenting organism.

In an preferred embodiment the acid concentration is maintained between 10 and 100 mmoles/L fermentation medium.

In a preferred embodiment the invention relates processes for producing fermentation products, such as ethanol, from starch-containing material comprising the steps of:

i) liquefying the starch-containing material at a temperature above the initial gelatinization temperature using:
an alpha-amylase derived from Bacillus stearothermophilus comprising a double deletion at positions I181+G182, and optionally a N193F substitution; (using SEQ ID NO: 1 for numbering);
   ii) saccharifying using a glucoamylase derived from a strain of Gloephyllum, such as Gloephyllum serpiarium or Gloephyllum trabeum.
   iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.

In a preferred embodiment the acid concentration is maintained between 10 and 100 mmoles/L fermentation medium.

In a preferred embodiment the invention relates processes for producing fermentation products, such as ethanol, from starch-containing material comprising the steps of:

i) liquefying the starch-containing material at a temperature above the initial gelatinization temperature using:
an alpha-amylase derived from Bacillus stearothermophilus;
a protease having a thermostability value of more than 20% determined as Relative Activity at 80° C./70° C., preferably derived from Pyrococcus furiosus and/or Thermoascus aurantiacus; and
optionally a Penicillium oxalicum glucoamylase;
   ii) saccharifying using a glucoamylase;
   iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.

In a preferred embodiment the acid concentration is maintained between 10 and 100 mmoles/L fermentation medium.

In a preferred embodiment the invention relates processes for producing fermentation products, such as ethanol, from starch-containing material comprising the steps of:

i) liquefying the starch-containing material at a temperature above the initial gelatinization temperature using:
an alpha-amylase, preferably derived from Bacillus stearothermophilus, comprising a double deletion at positions I181+G182, and optionally a N193F substitution (using SEQ ID NO: 1 for numbering) and having a T½ (min) at pH 4.5, 85° C., 0.12 mM CaCl₂ of at least 10;
   ii) saccharifying using a glucoamylase;
   iii) fermenting using a fermenting organism;
   wherein an acid having a pKa in the range from 3.75 to 5.75 is present or added in fermentation so that the acid concentration in fermentation is maintained between 10 and 100 mmoles/L fermentation medium and wherein the acid is added before the exponential growth phase of the fermenting organism.

In a preferred embodiment the invention relates processes for producing ethanol from starch-containing material comprising the steps of:
i) liquefying the starch-containing material at a temperature between 80-90° C.:
   an alpha-amylase, preferably derived from Bacillus stearothermophilus, having a T½ (min) at pH 4.5, 85° C., 0.12 mM CaCl₂ of at least 10;
   a protease, preferably derived from Pyrococcus furiosus and/or Thermoascus aurantiacus, having a thermostability value of more than 20% determined as Relative Activity at 80° C./70° C.;
   optionally a Penicillium oxalicum glucoamylase
ii) saccharifying using a glucoamylase;
iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.

In a preferred embodiment the acid concentration is maintained between 10 and 100 mmoles/L fermentation medium.

In a preferred embodiment the invention relates processes for producing fermentation products, such as ethanol from starch-containing material comprising the steps of:

i) liquefying the starch-containing material at a temperature above the initial gelatinization temperature using:
an alpha-amylase derived from Bacillus stearothermophilus having a double deletion at positions I181+G182, and optional substitution N193F; and optionally further one of the following set of substitutions:
E129V+K177L+R179E;
V59A+Q89R+E129V+K177L+R179E+H208Y+K220P+N224L+Q254S:
V59A+Q89R+E129V+K177L+R179E+Q254S+M284V;
V59A+E129V+K177L+R179E+Q254S+M284V;
E129V+K177L+R179E+K220P+N224L+S242Q+Q254S (using SEQ ID NO: 1 herein for numbering);
   ii) saccharifying using a glucoamylase, such as one from a strain of Gloephyllum, such as a strain of Gloephyllum serpiarium;
   iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.

In a preferred embodiment the acid concentration is maintained between 10 and 100 mmoles/L fermentation medium.

In a preferred embodiment the invention relates processes for producing fermentation products, such as ethanol, from starch-containing material comprising the steps of:

i) liquefying the starch-containing material at a temperature above the initial gelatinization temperature using:
an alpha-amylase derived from Bacillus stearothermophilus having a double deletion at positions I181+G182, and optional substitution N193F, and optionally further one of the following set of substitutions:
E129V+K177L+R179E;
V59A+Q89R+E129V+K177L+R179E+H208Y+K220P+N224L+Q254S:
V59A+Q89R+E129V+K177L+R179E+Q254S+M284V;
V59A+E129V+K177L+R179E+Q254S+M284V;
E129V+K177L+R179E+K220P+N224L+S242Q+Q254S (using SEQ ID NO: 1 herein for numbering).
a protease having a thermostability value of more than 20% determined as Relative Activity at 80° C./70° C., preferably derived from Pyrococcus furiosus and/or Thermoascus aurantiacus; and
optionally a Penicillium oxalicum glucoamylase shown in SEQ ID NO: 14 having substitutions selected from the group of:
K79V;
K79V+P11F+T65A+Q327F; or
K79V+P2N+P4S+P11F+T65A+Q327F; or
K79V+P11F+D26C+K33C+T65A+Q327F; or
K79V+P2N+P4S+P11F+T65A+Q327W+E501V+Y504T; or
K79V+P2N+P4S+P11F+T65A+Q327F+E501V+Y504T; or
K79V+P11F+T65A+Q327W+E501V+Y504T (using SEQ ID NO: 14 for numbering);
ii) saccharifying using a glucoamylase;
iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.

In an embodiment the acid concentration is maintained between 10 and 100 mmoles/L fermentation medium.

In a preferred embodiment the invention relates processes for producing fermentation products, such as ethanol from starch-containing material comprising the steps of:
i) liquefying the starch-containing material at a temperature between 80-90° C. using:
   an alpha-amylase derived from Bacillus stearothermophilus having a double deletion at positions I181+G182, and optional substitution N193F, and further optionally one of the following set of substitutions:
   E129V+K177L+R179E;
   V59A+Q89R+E129V+K177L+R179E+H208Y+K220P+N224L+Q254S;
   V59A+Q89R+E129V+K177L+R179E+Q254S+M284V;
   V59A+E129V+K177L+R179E+Q254S+M284V;
   E129V+K177L+R179E+K220P+N224L+S242Q+Q254S (using SEQ ID NO: 1 herein for numbering),
   a protease having a thermostability value of more than 20% determined as Relative Activity at 80° C./70° C., preferably derived from Pyrococcus furiosus and/or Thermoascus aurantiacus;
   a Penicillium oxalicum glucoamylase shown in SEQ ID NO: 14 having substitutions selected from the group of:
   K79V;
   K79V+P11F+T65A+Q327F; or
   K79V+P2N+P4S+P11F+T65A+Q327F; or
   K79V+P11F+D26C+K33C+T65A+Q327F; or
   K79V+P2N+P4S+P11F+T65A+Q327W+E501V+Y504T; or
   K79V+P2N+P4S+P11F+T65A+Q327F+E501V+Y504T; or
   K79V+P11F+T65A+Q327W+E501V+Y504T (using SEQ ID NO: 14 for numbering);
ii) saccharifying using a glucoamylase;
iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.

In a preferred embodiment the acid concentration is maintained between 10 and 100 mmoles/L fermentation medium.

In a preferred embodiment the invention relates processes for producing fermentation products, such as ethanol, from starch-containing material comprising the steps of:

i) liquefying the starch-containing material at a temperature above the initial gelatinization temperature using:
an alpha-amylase derived from Bacillus stearothermophilus having a double deletion at positions I181+G182, and optional substitution N193F;
a protease having a thermostability value of more than 20% determined as Relative Activity at 80° C./70° C., preferably derived from Pyrococcus furiosus and/or Thermoascus aurantiacus; and
optionally a pullulanase;
a Penicillium oxalicum glucoamylase having a K79V substilution (using SEQ ID NO: 14 herein for numbering);
   ii) saccharifying using a glucoamylase;
   iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.

In an embodiment the acid concentration is maintained between 10 and 100 mmoles/L fermentation medium.

In a preferred embodiment the invention relates processes for producing fermentation products, such as ethanol, from starch-containing material comprising the steps of:
i) liquefying the starch-containing material at a temperature above the initial gelatinization temperature using:
   an alpha-amylase, preferably derived from Bacillus stearothermophilus, having a T½ (min) at pH 4.5, 85° C., 0.12 mM CaCl₂ of at least 10;
   between 0.5 and 10 micro grams Pyrococcus furiosus protease per g DS;
ii) saccharifying using a glucoamylase selected from the group of glucoamylase derived from a strain of Aspergillus, preferably A. niger, A. awamori, or A. oryzae; or a strain of Trichoderma, preferably T. reesei; or a strain of Talaromyces, preferably T. emersonii, or a strain of Pycnoporus, or a strain of Gloephyllum, such as G. serpiarium or G. trabeum, or a strain of the Nigrofomes;
iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.

In a preferred embodiment the acid concentration is maintained between 10 and 100 mmoles/L fermentation medium.

In a preferred embodiment the invention relates processes for producing fermentation products, such as ethanol, from starch-containing material comprising the steps of:
i) liquefying the starch-containing material at a temperature between 80-90° C. using;
   an alpha-amylase, preferably derived from Bacillus stearothermophilus having a double deletion at positions I181+G182, and optional substitution N193F and having a T½ (min) at pH 4.5, 85° C., 0.12 mM CaCl₂ of at least 10;
   between 0.5 and 10 micro grams Pyrococcus furiosus protease per g DS;
   optionally a pullulanase;
   a Penicillium oxalicum glucoamylase;
ii) saccharifying using a glucoamylase;
iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.

In a preferred embodiment the acid concentration is maintained between 10 and 100 mmoles/L fermentation medium.

In a preferred embodiment the invention relates processes for producing fermentation products, such as ethanol, from starch-containing material comprising the steps of:
i) liquefying the starch-containing material at a temperature between 80-90° C. using;
   an alpha-amylase derived from Bacillus stearothermophilus having a double deletion I181+G182 and optional substitution N193F; and optionally further one of the following set of substitutions:
   E129V+K177L+R179E;
   V59A+Q89R+E129V+K177L+R179E+H208Y+K220P+N224L+Q254S;
   V59A+Q89R+E129V+K177L+R179E+Q254S+M284V:
   V59A+E129V+K177L+R179E+Q254S+M284V
   E129V+K177L+R179E+K220P+N224L+S242Q+Q254S (using SEQ ID NO: 1 herein for numbering);
   between 0.5 and 10 micro grams Pyrococcus furiosus protease per g DS; and
   optionally a pullulanase;
   a Penicillium oxalicum glucoamylase shown in SEQ ID NO: 14 having substitutions selected from the group of:
   K79V;
   K79V+P11F+T65A+Q327F; or
   K79V+P2N+P4S+P11F+T65A+Q327F; or
   K79V+P11F+D26C+K33C+T65A+Q327F; or
   K79V+P2N+P4S+P11F+T65A+Q327W+E501V+Y504T; or
   K79V+P2N+P4S+P11F+T65A+Q327F+E501V+Y504T; or
   K79V+P11F+T65A+Q327W+E501V+Y504T (using SEQ ID NO: 14 for numbering);
ii) saccharifying using a glucoamylase;
iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.

In an embodiment the acid concentration is maintained between 10 and 100 mmoles/L fermentation medium.

In a preferred embodiment the invention relates processes for producing fermentation products, such as ethanol, from starch-containing material comprising the steps of:
i) liquefying the starch-containing material at a temperature between 80-90° C. using:
   an alpha-amylase derived from Bacillus stearothermophilus having a double deletion I181+G182 and optional substitution N193F; and further one of the following set of substitutions:
   E129V+K177L+R179E;
   V59A+Q89R+E129V+K177L+R179E+H208Y+K220P+N224L+Q254S;
   V59A+Q89R+E129V+K177L+R179E+Q254S+M284V;
   V59A+E129V+K177L+R179E+Q254S+M284V
   E129V+K177L+R179E+K220P+N224L+S242Q+Q254S (using SEQ ID NO: 1 herein for numbering).
   a protease having a thermostability value of more than 20% determined as Relative Activity at 80° C./70° C., preferably derived from Pyrococcus furiosus and/or Thermoascus aurantiacus; and
   optionally a pullulanase;
   a Penicillium oxalicum glucoamylase shown in SEQ ID NO: 14 having substitutions selected from the group of:
   K79V;
   K79V+P11F+T65A+Q327F; or
   K79V+P2N+P4S+P11F+T65A+Q327F; or
   K79V+P11F+D26C+K33C+T65A+Q327F; or
   K79V+P2N+P4S+P11F+T65A+Q327W+E501V+Y504T; or
   K79V+P2N+P4S+P11F+T65A+Q327F+E501V+Y504T; or
   K79V+P11F+T65A+Q327W+E501V+Y504T (using SEQ ID NO: 14 for numbering);
ii) saccharifying using a glucoamylase selected from the group of glucoamylase derived from a strain of Aspergillus; or a strain of Trichoderma; a strain of Talaromyces, a strain of Pycnoporus; a strain of Gloephyllum; and a strain of the Nigrofomes;
iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.

In a preferred embodiment the acid concentration is maintained between 10 and 100 mmoles/L fermentation medium.

In a preferred embodiment the invention relates processes for producing fermentation products, such as ethanol, from starch-containing material comprising the steps of:
i) liquefying the starch-containing material at a temperature between 80-90° C. at a pH between 5.0 and 6.5 using:
   an alpha-amylase derived from Bacillus stearothermophilus having a double deletion I181+G182 and optional substitution N193F; and optionally further one of the following set of substitutions:
   E129V+K177L+R179E;
   V59A+Q89R+E129V+K177L+R179E+H208Y+K220P+N224L+Q254S;
   V59A+Q89R+E129V+K177L+R179E+Q254S+M284V;
   V59A+E129V+K177L+R179E+Q254S+M284V
   E129V+K177L+R179E+K220P+N224L+S242Q+Q254S (using SEQ ID NO: 1 herein for numbering).
   a protease derived from Pyrococcus furiosus, preferably the one shown in SEQ ID NO: 13 herein;
   a Penicillium oxalicum glucoamylase shown in SEQ ID NO: 14 having substitutions selected from the group of:
   K79V;
   K79V+P11F+T65A+Q327F; or
   K79V+P2N+P4S+P11F+T65A+Q327F; or
   K79V+P11F+D26C+K33C+T65A+Q327F; or
   K79V+P2N+P4S+P11F+T65A+Q327W+E501V+Y504T; or
   K79V+P2N+P4S+P11F+T65A+Q327F+E501V+Y504T; or
   K79V+P11F+T65A+Q327W+E501V+Y504T (using SEQ ID NO: 14 for numbering);
ii) saccharifying using a glucoamylase;
iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.

In a preferred embodiment the acid concentration is maintained between 10 and 100 mmoles/L fermentation medium.

In a preferred embodiment the invention relates processes for producing ethanol from starch-containing material comprising the steps of:

i) liquefying the starch-containing material at a temperature between 80-90° C. at a pH between 5.0 and 6.5 using:
an alpha-amylase derived from Bacillus stearothermophilus having a double deletion I181+G182 and optional substitution N193F; and optionally further one of the following set of substitutions:
E129V+K177L+R179E;
V59A+Q89R+E129V+K177L+R179E+H208Y+K220P+N224L+Q254S;
V59A+Q89R+E129V+K177L+R179E+Q254S+M284V;
V59A+E129V+K177L+R179E+Q254S+M284V
E129V+K177L+R179E+K220P+N224L+S242Q+Q254S (using SEQ ID NO: 1 herein for numbering).
a protease derived from Pyrococcus furiosus, preferably the one shown in SEQ ID NO: 13 herein;
a Penicillium oxalicum glucoamylase shown in SEQ ID NO: 14 having substitutions selected from the group of:
K79V;
K79V+P11F+T65A+Q327F; or
K79V+P2N+P4S+P11F+T65A+Q327F; or
K79V+P11F+D26C+K33C+T65A+Q327F; or
K79V+P2N+P4S+P11F+T65A+Q327W+E501V+Y504T; or
K79V+P2N+P4S+P11F+T65A+Q327F+E501V+Y504T; or
K79V+P11F+T65A+Q327W+E501V+Y504T (using SEQ ID NO: 14 for numbering);
   ii) saccharifying using a glucoamylase;
   iii) fermenting using a strain of Saccharomyces cerevisiae, such as ETHANOL RED™;
   wherein an acid selected from the group of acetic acid, benzoic acid, propionic acid, sorbic acid, formic acid, and succinic acid is present or added in fermentation so that the acid concentration in fermentation is maintained between above 0 (zero) and 100 mmoles/L fermentation medium and wherein the acid is added before the exponential growth phase of the fermenting organism.

In a preferred embodiment the acid concentration is maintained between 10 and 100 mmoles/L fermentation medium.

### Raw Starch Hydrolysis Processes of the Invention

A process for producing ethanol according to this aspect of the invention is carried out as a raw starch hydrolysis (RSH) process. A raw starch hydrolysis process is a process where starch, typically granular starch, is converted into dextrins/sugars by raw starch degrading enzymes at temperatures below the initial gelatinization temperature of the starch in question and converted into ethanol by yeast, typically of Saccharomyces cerevisiae. This type of process is often alternatively referred to as a “one-step process” or “no cook” process.

Specifically, the invention relates to processes for producing a fermentation product from starch-containing material comprising the steps of:

(i) saccharifying the starch-containing material at a temperature below the initial gelatinization temperature
(ii) fermenting using a fermenting organism;
wherein saccharification and/or fermentation is done in the presence of the following enzymes: glucoamylase and alpha-amylase, and optionally protease; and wherein an acid having a pKa in the range from 3.75 to 5.75 is present and/or added in fermentation so that the acid concentration in fermentation is maintained between above 0 (zero) and 100 mmoles/L fermentation medium and wherein the acid is added before the exponential growth phase of the fermenting organism.

In a preferred embodiment saccharification and fermentation are carried out simultaneosly (one step process). However, step (a) and step (b) may also be carried our sequentially.

In a preferred embodiment the acid concentration is maintained between 10 and 100 mmoles/L fermentation medium. In a preferred embodiment the acid concentration is maintained between 5 and 80 mmoles/L fermentation medium.

In processes of the invention the starch does not gelatinize as the process is carried out at temperatures below the initial gelatinization temperature of the starch in question.

The term “initial gelatinization temperature” means the lowest temperature at which starch gelatinization commences. In general, starch heated in water begins to gelatinize between about 50° C. and 75° C. The exact temperature of gelatinization depends on the specific starch and depends on the degree of cross-linking of the amylopectin. The initial gelatinization temperature can readily be determined by the skilled artisan. The initial gelatinization temperature may vary according to the plant species, to the particular variety of the plant species as well as with the growth conditions. In context of this invention the initial gelatinization temperature of a given starch-containing material may be determined as the temperature at which birefringence is lost in 5% of the starch granules using the method described by Gorinstein. S. and Lii. C., Starch/Starke, Vol. 44 (12) pp. 461-466 (1992).

Therefore, according to the process of the invention ethanol is produced from un-gelatinized (i.e., uncooked), preferably milled grains, such as corn, or small grains such as wheat, oats, barley, rye, rice, or cereals such as sorghum. Examples of suitable starch-containing starting materials are listed in the section “Starch-Containing Materials”-section below.

In a preferred embodiment the enzymes may be added as one or more enzyme blends. According to the invention the fermentation product, i.e., ethanol, is produced without liquefying the starch-containing material. The process of the invention includes saccharifying (e.g., milled) starch-containing material, especially granular starch, below the initial gelatinization temperature, in the presence of at least a glucoamylase and an alpha-amylase and optionally a protease and/or a cellulolytic enzyme composition. The dextrins/sugars generated during saccharification can may according to the invention be simultaneously fermented into ethanol by a suitable fermenting organism, especially Saccharomyces cerevisiae.

Before step (i) an aqueous slurry of starch-containing material, such as granular starch, having 10-55 wt.-% dry solids (DS), preferably 25-45 wt.-% dry solids, more preferably 30-40% dry solids of starch-containing material may be prepared. The slurry may include water and/or process waters, such as stillage (backset), scrubber water, evaporator condensate or distillate, side-stripper water from distillation, or process water from other fermentation product plants. Because the process of the invention is carried out below the initial gelatinization temperature and thus no significant viscosity increase takes place, high levels of stillage may be used, if desired. In an embodiment the aqueous slurry contains from about 1 to about 70 vol.-%, preferably 15-60% vol.-%, especially from about 30 to 50 vol.-% water and/or process waters, such as stillage (backset), scrubber water, evaporator condensate or distillate, side-stripper water from distillation, or process water from other fermentation product plants, or combinations thereof, or the like.

In an embodiment backset, or another recycled stream, is added to the slurry before step (i), or to the saccharification (step (i)), or to the simultaneous saccharification and fermentation steps (combined step (i) and step (ii)).

After being subjected to a process of the invention at least 85%, at least 86%, at least 87%, at least 88%, at least 89%, at least 90%, at least 91%, at least 92%, at least 93%, at least 94%, at least 95%, at least 96%, at least 97%, at least 98%, or preferably at least 99% of the dry solids in the starch-containing material are converted into a soluble starch hydrolysate.

A process of the invention is conducted at a temperature below the initial gelatinization temperature, which means that the temperature at which a separate step (i) is carried out typically lies in the range between 25-75° C., such as between 30-70° C., or between 45-60° C.

In a preferred embodiment the temperature during fermentation in step (b) or simultaneous saccharification and fermentation in steps (i) and (ii) is between 25° C. and 40° C., preferably between 28° C. and 36° C., such as between 28° C. and 35° C., such as between 28° C. and 34° C., such as around 32° C.

In an embodiment of the invention fermentation or SSF is carried out for 30 to 150 hours, preferably 48 to 96 hours.

In an embodiment fermentation or SSF is carried out so that the sugar level, such as glucose level, is kept at a low level, such as below 6 wt.-%, such as below about 3 wt.-%, such as below about 2 wt.-%, such as below about 1 wt.-%., such as below about 0.5%, or below 0.25% wt.-%, such as below about 0.1 wt.-%. Such low levels of sugar can be accomplished by simply employing adjusted quantities of enzymes and fermenting organism. A skilled person in the art can easily determine which doses/quantities of enzyme and fermenting organism to use. The employed quantities of enzyme and fermenting organism may also be selected to maintain low concentrations of maltose in the fermentation broth. For instance, the maltose level may be kept below about 0.5 wt.-%, such as below about 0.2 wt.-%.

The process of the invention may be carried out at a pH from 3 and 7, preferably from 3 to 6, or more preferably from 3.5 to 5.0.

The term “granular starch” means raw uncooked starch, i.e., starch in its natural form found in, e.g., cereal, tubers or grains. Starch is formed within plant cells as tiny granules insoluble in water. When put in cold water, the starch granules may absorb a small amount of the liquid and swell. At temperatures up to around 50° C. to 75° C. the swelling may be reversible. However, at higher temperatures an irreversible swelling called “gelatinization” begins. The granular starch may be a highly refined starch, preferably at least 90%, at least 95%, at least 97% or at least 99.5% pure, or it may be a more crude starch-containing materials comprising (e.g., milled) whole grains including non-starch fractions such as germ residues and fibers.

The raw material, such as whole grains, may be reduced in particle size, e.g., by milling, in order to open up the structure and allowing for further processing. Examples of suitable particle sizes are disclosed in U.S. Pat. No. 4,514,496 and WO2004/081193 (incorporated by reference). Two processes are preferred according to the invention: wet and dry milling. In dry milling whole kernels are milled and used. Wet milling gives a good separation of germ and meal (starch granules and protein) and is often applied at locations where the starch hydrolysate is used in production of, e.g., syrups. Both dry and wet milling is well known in the art of starch processing.

In an embodiment the particle size is reduced to between 0.05 to 3.0 mm, preferably 0.1-0.5 mm, or so that at least 30%, preferably at least 50%, more preferably at least 70%, even more preferably at least 90% of the starch-containing material fit through a sieve with a 0.05 to 3.0 mm screen, preferably 0.1-0.5 mm screen. In a preferred embodiment starch-containing material is prepared by reducing the particle size of the starch-containing material, preferably by milling, such that at least 50% of the starch-containing material has a particle size of 0.1-0.5 mm.

According to the invention the enzymes are added so that the glucoamylase is present in an amount of 0.001 to 10 AGU/g DS, preferably from 0.01 to 5 AGU/g DS, especially 0.1 to 0.5 AGU/g DS.

According to the invention the enzymes are added so that the alpha-amylase is present or added in an amount of 0.001 to 10 AFAU/g DS, preferably from 0.01 to 5 AFAU/g DS, especially 0.3 to 2 AFAU/g DS or 0.001 to 1 FAU-F/g DS, preferably 0.01 to 1 FAU-F/g DS.

According to the invention the enzymes are added so that the cellulolytic enzyme composition is present or added in an amount 1-10,000 micro grams EP/g DS, such as 2-5,000, such as 3 and 1,000, such as 4 and 500 micro grams EP/g DS.

According to the invention the enzymes are added so that the cellulolytic enzyme composition is present or added in an amount in the range from 0.1-100 FPU per gram total solids (TS), preferably 0.5-50 FPU per gram TS, especially 1-20 FPU per gram TS.

In an embodiment of the invention the enzymes are added so that the protease is present in an amount of 0.0001-1 mg enzyme protein per g DS, preferably 0.001 to 0.1 mg enzyme protein per g DS. Alternatively, the protease is present and/or added in an amount of 0.0001 to 1 LAPU/g DS, preferably 0.001 to 0.1 LAPU/g DS and/or 0.0001 to 1 mAU-RH/g DS, preferably 0.001 to 0.1 mAU-RH/g DS.

In an embodiment of the invention the enzymes are added so that the protease is present or added in an amount in the range 1-1,000 μg EP/g DS, such as 2-500 μg EP/g DS, such as 3-250 μg EP/g DS.

In a preferred embodiment ratio between glucoamylase and alpha-amylase is between 99:1 and 1:2, such as between 98:2 and 1:1, such as between 97:3 and 2:1, such as between 96:4 and 3:1, such as 97:3, 96:4, 95:5, 94:6, 93:7, 90:10, 85:15, 83:17 or 65:35 (mg EP glucoamylase: mg EP alpha-amylase).

In a preferred embodiment the total dose of glucoamylase and alpha-amylase is according to the invention from 10-1,000 μg/g DS, such as from 50-500 μg/g DS, such as 75-250 μg/g DS.

In a preferred embodiment the total dose of cellulolytic enzyme composition added is from 10-500 μg/g DS, such as from 20-400 μg/g DS, such as 20-300 μg/g DS.

In an embodiment the dose of protease added is from 1-200 μg/g DS, such as from 2-100 μg/g DS, such as 3-50 μg/g DS.

In a preferred embodiment the glucoamylase is a Gloeophyllum glucoamylase, preferably Gloeophyllum trabeum glucoamylase. In a preferred embodiment the glucoamylase is the Gloeophyllum trabeum glucoamylase shown in SEQ ID NO: 17 herein. In an embodiment the Gloeophyllum trabeum glucoamylase shown in SEQ ID NO: 17 has one of the following substitutions: V59A; S95P; A121P; T119W; S95P+A121P; V59A-F595P; S95P+T119W; V59A+S95P+A121P; or S95P+T119W+A121P, especially S95P+A121P.

In another embodiment the glucoamylase is a Trametes glucoamylase, preferably Trametes cingulata glucoamylase. In a preferred embodiment the glucoamylase is the Trametes cingulata glucoamylase shown in SEQ ID NO: 20 herein.

In an embodiment glucoamylase is selected from the group consisting of:

(i) a glucoamylase comprising the mature polypeptide of SEQ ID NO: 20 herein;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the mature polypeptide of SEQ ID NO: 20 herein.

In a preferred embodiment the alpha-amylase is derived from Rhizomucor pusillus, preferably with an Aspergillus niger glucoamylase linker and starch-binding domain (SBD), preferably the one disclosed as V039 in Table 5 in WO 2006/069290 or SEQ ID NO: 16 herein.

In a preferred embodiment the glucoamylase is the Trametes cingulata glucoamylase shown in SEQ ID NO: 20 herein and the alpha-amylase is Rhizomucor pusillus alpha-amylase with an Aspergillus niger glucoamylase linker and starch-binding domain (SBD).

In an embodiment the alpha-amylase is derived from Rhizomucor pusillus.

In an embodiment the glucoamylase, such as one derived from Gloeophyllum trabeum, is selected from the group consisting of:

(i) a glucoamylase comprising the mature polypeptide of SEQ ID NO: 17 herein;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the mature polypeptide of SEQ ID NO: 17 herein.

In an embodiment the alpha-amylase is a Rhizomucor pusillus alpha-amylase with an Aspergillus niger glucoamylase linker and starch-binding domain (SBD), preferably one having at least one of the following substitutions or combinations of substitutions: D165M; Y141W; Y141R; K136F; K192R; P224A; P224R; S123H+Y141W; G20S+Y141W; A76G+Y141W; G128D+Y141W; G128D+D143N; P219C+Y141W; N142D+D143N; Y141W+K192R; Y141W+D143N; Y141W+N383R; Y141W+P219C+A265C; Y141W+N142D+D143N; Y141W+K192R V410A; G128D+Y141W+D143N; Y141W+D143N+P219C; Y141W+D143N+K192R; G128D+D143N+K192R; Y141W+D143N+K192R+P219C; G128D+Y141W+D143N+K192R; or G128D+Y141W+D143N+K192R+P219C, especially G128D+D143N (using SEQ ID NO: 16 herein for numbering).

In an embodiment the glucoamylase is the Gloeophyllum trabeum glucoamylase shown in SEQ ID NO: 17 herein having one of the following substitutions: S95P+A121P and the alpha-amylase is is Rhizomucor pusillus alpha-amylase with an Aspergillus niger glucoamylase linker and starch-binding domain (SBD), preferably one having the following substitutions G128D+D143N (using SEQ ID NO: 16 herein for numbering).

In another embodiment the glucoamylase is the Pycnoporus sanguineus glucoamylase shown in SEQ ID NO: 18 herein. In an embodiment the glucoamylase, such as one from Pycnoporus sanguineus, is selected from the group consisting of:

(i) a glucoamylase comprising the mature polypeptide of SEQ ID NO: 18 herein;
(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the mature polypeptide of SEQ ID NO: 18 herein.

In an embodiment the glucoamylase is the Pycnoporus sanguineus glucoamylase shown in SEQ ID NO: 18 herein, and the alpha-amylase is the Rhizomucor pusillus with an Aspergillus niger glucoamylase linker and starch-binding domain (SBD), preferably the one disclosed as V039 in Table 5 in WO 2006/069290 or SEQ ID NO: 16 herein, preferably one having one or more of the following substitutions: G128D, D143N, especially G128D+D143N.

In a preferred embodiment the ratio between glucoamylase and alpha-amylase is between 99:1 and 1:2, such as between 98:2 and 1:1, such as between 97:3 and 2:1, such as between 96:4 and 3:1, such as 97:3, 96:4, 95:5, 94:6, 93:7, 90:10, 85:15, 83:17 or 65:35 (mg EP glucoamylase: mg EP alpha-amylase).

In an embodiment the total dose of glucoamylase and alpha-amylase added is from 10-1,000 μg/g DS, such as from 50-500 μg/g DS, such as 75-250 μg/g DS.

In an embodiment a protease is present and/or added during fermentation or simultaneous saccharification step (i) and fermentation step (ii). In an embodiment the dose of protease added is from 1-200 μg/g DS, such as from 2-100 μg/g DS, such as 3-50 μg/g DS.

In an embodiment a cellulolytic enzyme composition is present and/or added during fermentation or simultaneous saccharification step (i) and fermentation step (ii). In an embodiment the total dose of cellulolytic enzyme composition added is from 10-500 μg/g DS, such as from 20-400 μg/g DS, such as 20-300 μg/g DS.

### Examples of Preferred Raw Starch Hydrolysis Processes of the Invention

In a preferred embodiment the invention relates to the processes for producing a fermentation product, preferably ethanol, from starch-containing material comprising the steps of:

(i) saccharifying the starch-containing material at a temperature below the initial gelatinization temperature
(ii) fermenting using a fermenting organism;
wherein saccharification and/or fermentation is done in the presence of the following enzymes: glucoamylase and alpha-amylase, and optionally protease; and wherein an acid having a pKa in the range from 3.75 to 5.75 is present and/or added in fermentation so that the acid concentration in fermentation is maintained between above 0 (zero) and 100 mmoles/L fermentation medium and wherein the acid is added before the exponential growth phase of the fermenting organism;
   wherein the glucoamylase is a Gloeophyllum trabeum glucoamylase, preferably one having one of the following substitutions: V59A; S95P; A121P; T119W; S95P+A121P; V59A+S95P; S95P+T119W; V59A+S95P+A121P); or S95P+T119W+A121P, especially S95P+A121P (using SEQ ID NO: 17 herein for numbering); and the alpha-amylase is preferably an alpha-amylase derived from Rhizomucor pusillus, preferably with an Aspergillus niger glucoamylase linker and starch-binding domain (SBD), preferably the one disclosed as V039 in Table 5 in WO 2006/069290 or SEQ ID NO: 16 herein, preferably one having at least one of the following substitutions or combinations of substitutions: D165M; Y141W; Y141R; K136F; K192R; P224A; P224R; S123H+Y141W; G20S+Y141W; A76G+Y141W; G128D+Y141W; G128D+D143N; P219C+Y141W; N142D+D143N; Y141W+K192R; Y141W+D143N; Y141W+N383R; Y141W+P219C+A265C; Y141W+N142D+D143N; Y141W+K192R V410A; G128D+Y141W+D143N; Y141W+D143N+P219C; Y141W+D143N+K192R; G128D+D143N+K192R; Y141W+D143N+K192R+P219C: G128D+Y141W+D143N+K192R; or G128D+Y141W+D143N+K192R+P219C, especially G128D+D143N (using SEQ ID NO: 16 herein for numbering).

In a preferred embodiment the invention relates to processes for producing a fermentation product, preferably ethanol, from starch-containing material comprising the steps of:

(i) saccharifying the starch-containing material at a temperature below the initial gelatinization temperature
(ii) fermenting using a fermenting organism;
wherein saccharification and/or fermentation is done in the presence of the following enzymes: glucoamylase and alpha-amylase, and optionally protease; and wherein an acid having a pKa in the range from 3.75 to 5.75 is present and/or added in fermentation so that the acid concentration in fermentation is maintained between above 0 (zero) and 100 mmoles/L fermentation medium and wherein the acid is added before the exponential growth phase of the fermenting organism;
   wherein the glucoamylase is a Trametes cingulata glucoamylase; and the alpha-amylase is preferably derived from Rhizomucor pusillus, preferably with an Aspergillus niger glucoamylase linker and starch-binding domain (SBD), preferably the one disclosed as V039 in Table 5 in WO 2006/069290 or SEQ ID NO: 16 herein, preferably one having at least one of the following substitutions or combinations of substitutions: D165M; Y141W; Y141R; K136F; K192R; P224A; P224R; S123H+Y141W; G20S+Y141W; A76G+Y141W; G128D+Y141W; G128D+D143N; P219C+Y141W; N142D+D143N; Y141W+K192R; Y141W+D143N; Y141W+N383R; Y141W+P219C+A265C; Y141W+N142D+D143N; Y141W+K192R V410A; G128D+Y141W+D143N; Y141W+D143N+P219C; Y141W+D143N+K192R; G128D+D143N+K192R; Y141W+D143N+K192R+P219C; G128D+Y141W+D143N+K192R; or G128D+Y141W+D143N+K192R+P219C, especially G128D+D143N (using SEQ ID NO: 16 herein for numbering).

In a preferred embodiment the invention relates to processes for producing a fermentation product, preferably ethanol, from starch-containing material comprising the steps of:

(i) saccharifying the starch-containing material at a temperature below the initial gelatinization temperature;
(ii) fermenting using a fermenting organism;
wherein saccharification and/or fermentation is done in the presence of the following enzymes: glucoamylase and alpha-amylase, and optionally protease; and wherein an acid having a pKa in the range from 3.75 to 5.75 is present and/or added in fermentation so that the acid concentration in fermentation is maintained between above 0 (zero) and 100 mmoles/L fermentation medium and wherein the acid is added before the exponential growth phase of the fermenting organism;
   wherein the glucoamylase is a Pycnoporus sanguineus glucoamylase; and the alpha-amylase is preferably an alpha-amylase derived from Rhizomucor pusillus, preferably with an Aspergillus niger glucoamylase linker and starch-binding domain (SBD), preferably the one disclosed as V039 in Table 5 in WO 2006/069290 or SEQ ID NO: 16 herein, preferably one having at least one of the following substitutions or combinations of substitutions: D165M; Y141W; Y141R; K136F; K192R; P224A; P224R; S123H+Y141W; G20S+Y141W; A76G+Y141W; G128D+Y141W; G128D+D143N; P219C+Y141W; N142D+D143N; Y141W+K192R; Y141W+D143N; Y141W+N383R; Y141W+P219C+A265C; Y141W+N142D+D143N; Y141W+K192R V410A; G128D+Y141W+D143N; Y141W+D143N+P219C; Y141W+D143N+K192R; G128D+D143N+K192R; Y141W+D143N+K192R+P219C; G128D+Y141W+D143N+K192R; or G128D+Y141W+D143N+K192R+P219C, especially G128D+D143N (using SEQ ID NO: 16 herein for numbering).

### Materials & Methods

### Materials:

Alpha-Amylase A (“AAA”): Bacillus stearothermophilus alpha-amylase with the mutations I181*+G182*+N193F truncated to 491 amino acids (using SEQ ID NO: 1 herein for numbering)
Protease Pfu (“PFU”): Protease derived from Pyrococcus furiosus shown in SEQ ID NO: 13 herein.
PsAMG: Glucoamylase derived from Pycnoporus sanguineus disclosed as shown in SEQ ID NO: 4 in WO 2011/066576 and in SEQ ID NO: 18 herein.
TcAMG: Glucoamylase derived from Trametes cingulata shown in SEQ ID NO: 19 herein or SEQ ID NO: 2 in WO 2006/69289.
JA126: Alpha-amylase derived from Rhizomucor pusillus with an Aspergillus niger glucoamylase linker and starch-binding domain (SBD) shown in SEQ ID NO: 16 herein.
AAPE096: Alpha-amylase derived from Rhizomucor pusillus with an Aspergillus niger glucoamylase linker and starch-binding domain (SBD) shown in SEQ ID NO: 16 herein, with the following substitutions: G128D+D143N.
RSH Blend P: Blend of TcAMG and JA126 with a ratio between AGU (from TcAMG) and FAU-F (JA126) of about 10:1.
Glucoamylase SA (“GSA”) comprises a blend comprising Talaromyces emersonii glucoamylase disclosed in WO99/28448 (SEQ ID NO: 19 herein), Trametes cingulata glucoamylase disclosed as SEQ ID NO: 2 in WO 06/69289 and SEQ ID NO: 20 herein, and Rhizomucor pusillus alpha-amylase with Aspergillus niger glucoamylase linker and SBD disclosed as SEQ ID NO: 16 herein with the following substitutions: G128D+D143N (activity ratio AGU:AGU:FAU(F): approx. 30:7:1).
Cellulase VD (“CVD”): Cellulolytic composition derived from Trichoderma reesei comprising GH61A polypeptide having cellulolytic enhancing activity derived from a strain of Penicillium emersonii (SEQ ID NO: 2 in WO 2011/041397), Aspergillus fumigatus beta-glucosidase variant (SEQ ID NO: 2 in WO 2005/047499 with the following substitutions: F100D, S283G, N456E, F512Y) disclosed in WO 2012/044915; Aspergillus fumigatus Cel7A CBH1 disclosed as SEQ ID NO: 6 in WO2011/057140 and Aspergillus fumigatus CBH II disclosed as SEQ ID NO: 18 in WO 2011/057140.

### Yeast:

ETHANOL RED™ (“ER”): Saccharomyces cerevisiae yeast available from Fermentis/Lesaffre, USA.

### Methods

### Identity:

The relatedness between two amino acid sequences or between two nucleotide sequences is described by the parameter “identity”.

For purposes of the present invention the degree of identity between two amino acid sequences, as well as the degree of identity between two nucleotide sequences, may be determined by the program “align” which is a Needleman-Wunsch alignment (i.e. a global alignment). The program is used for alignment of polypeptide, as well as nucleotide sequences. The default scoring matrix BLOSUM50 is used for polypeptide alignments, and the default identity matrix is used for nucleotide alignments. The penalty for the first residue of a gap is −12 for polypeptides and −16 for nucleotides. The penalties for further residues of a gap are −2 for polypeptides, and −4 for nucleotides.

“Align” is part of the FASTA package version v20u6 (see W. R. Pearson and D. J. Lipman (1988), “Improved Tools for Biological Sequence Analysis”, PNAS 85:2444-2448, and W. R. Pearson (1990) “Rapid and Sensitive Sequence Comparison with FASTP and FASTA,”

Methods in Enzymology 183:63-98). FASTA protein alignments use the Smith-Waterman algorithm with no limitation on gap size (see “Smith-Waterman algorithm”, T. F. Smith and M. S. Waterman (1981) J. Mol. Biol. 147:195-197).

### Protease Assays

### AZCL-Casein Assay

A solution of 0.2% of the blue substrate AZCL-casein is suspended in Borax/NaH₂PO₄ buffer pH9 while stirring. The solution is distributed while stirring to microtiter plate (100 microL to each well), 30 microL enzyme sample is added and the plates are incubated in an Eppendorf Thermomixer for 30 minutes at 45° C. and 600 rpm. Denatured enzyme sample (100° C. boiling for 20 min) is used as a blank. After incubation the reaction is stopped by transferring the microtiter plate onto ice and the coloured solution is separated from the solid by centrifugation at 3000 rpm for 5 minutes at 4° C. 60 microL of supernatant is transferred to a microtiter plate and the absorbance at 595 nm is measured using a BioRad Microplate Reader.

pNA-Assay

50 microL protease-containing sample is added to a microtiter plate and the assay is started by adding 100 microL 1 mM pNA substrate (5 mg dissolved in 100 microL DMSO and further diluted to 10 mL with Borax/NaH₂PO₄ buffer pH 9.0). The increase in OD₄₀₅ at room temperature is monitored as a measure of the protease activity.

### Glucoamylase Activity (AGU)Glucoamylase Activity May be Measured in Glucoamylase Units (Agu).

The Novo Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

| AMG incubation: | |
|---|---|
| Substrate: | maltose 23.2 mM |
| Buffer: | acetate 0.1M |
| pH: | 4.30 ± 0.05 |
| Incubation temperature: | 37° C. ± 1 |
| Reaction time: | 5 minutes |
| Enzyme working range: | 0.5-4.0 AGU/mL |

| Color reaction: | |
|---|---|
| GlucDH: | 430 U/L |
| Mutarotase: | 9 U/L |
| NAD: | 0.21 mM |
| Buffer: | phosphate 0.12M; 0.15M NaCl |
| pH: | 7.60 ± 0.05 |
| Incubation temperature: | 37° C. ± 1 |
| Reaction time: | 5 minutes |
| Wavelength: | 340 nm |

A folder (EB-SM-0131.02/01) describing this analytical method in more detail is available on request from Novozymes NS, Denmark, which folder is hereby included by reference.

### Acid Alpha-Amylase Activity (AFAU)

Acid alpha-amylase activity may be measured in AFAU (Acid Fungal Alpha-amylase Units), which are determined relative to an enzyme standard. 1 AFAU is defined as the amount of enzyme which degrades 5.260 mg starch dry matter per hour under the below mentioned standard conditions.

Acid alpha-amylase, an endo-alpha-amylase (1,4-alpha-D-glucan-glucanohydrolase, E.C. 3.2.1.1) hydrolyzes alpha-1,4-glucosidic bonds in the inner regions of the starch molecule to form dextrins and oligosaccharides with different chain lengths. The intensity of color formed with iodine is directly proportional to the concentration of starch. Amylase activity is determined using reverse colorimetry as a reduction in the concentration of starch under the specified analytical conditions.

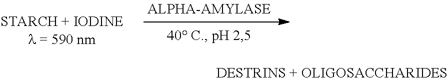

| | | |
|---|---|---|
| blue/violet | t = 23 sec. | decoloration |

Standard conditions/reaction conditions:
Substrate: Soluble starch, approx. 0.17 g/L
Buffer: Citrate, approx. 0.03 M
Iodine (12): 0.03 g/L
CaCl2: 1.85 mM
pH: 2.50±0.05
Incubation temperature: 40° C.
Reaction time: 23 seconds
Wavelength: 590 nm
Enzyme concentration: 0.025 AFAU/mL
Enzyme working range: 0.01-0.04 AFAU/mL

A folder EB-SM-0259.02/01 describing this analytical method in more detail is available upon request to Novozymes NS, Denmark, which folder is hereby included by reference.

### Alpha-Amylase Activity (KNU)

The alpha-amylase activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e., at 37° C.+/−0.05; 0.0003 M Ca²⁺; and pH 5.6) dextrinizes 5260 mg starch dry substance Merck Amylum solubile.

A folder EB-SM-0009.02/01 describing this analytical method in more detail is available upon request to Novozymes NS, Denmark, which folder is hereby included by reference.

### Alpha-Amylase Activity (KNU-A)

Alpha amylase activity is measured in KNU(A) Kilo Novozymes Units (A), relative to an enzyme standard of a declared strength.

Alpha amylase in samples and α-glucosidase in the reagent kit hydrolyze the substrate (4,6-ethylidene(G₇)-p-nitrophenyl(G₁)-α,D-maltoheptaoside (ethylidene-G₇PNP) to glucose and the yellow-colored p-nitrophenol.

The rate of formation of p-nitrophenol can be observed by Konelab 30. This is an expression of the reaction rate and thereby the enzyme activity.

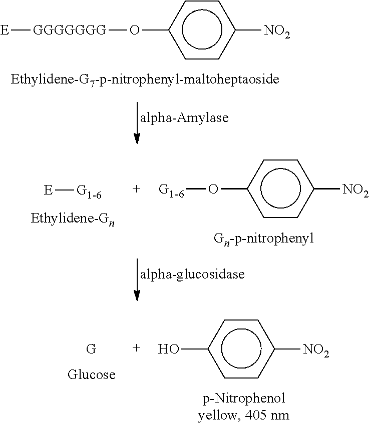

The enzyme is an alpha-amylase with the enzyme classification number EC 3.2.1.1.

| Parameter | Reaction conditions |
|---|---|
| Temperature | 37° C. |
| pH | 7.00 (at 37° C.) |
| Substrate conc. | Ethylidene-G₇PNP, R2: 1.86 mM |
| Enzyme conc. (conc. of high/low | 1.35-4.07 KNU(A)/L |
| standard in reaction mixture) | |
| Reaction time | 2 min |
| Interval kinetic measuring time | 7/18 sec. |
| Wave length | 405 nm |
| Conc. of reagents/chemicals critical | α-glucosidase, R1: ≧3.39 kU/L |
| for the analysis | |

A folder EB-SM-5091.02-D on determining KNU-A activity is available upon request to Novozymes NS, Denmark, which folder is hereby included by reference.

### Alpha-Amylase Activity KNU(S)

BS-amylase in samples and the enzyme alpha-glucosidase in the reagent kit hydrolyze substrate (4,6-ethylidene(G7)-p-nitrophenyl(G1)-alpha-D-maltoheptaoside (ethylidene-G7PNP)) to glucose and the yellow-colored p-nitrophenol.

The rate of formation of p-nitrophenol can be observed by Konelab 30. This is an expression of the reaction rate and thereby the enzyme activity.

Reaction Conditions

Reaction conditions

| | |
|---|---|
| Reaction: | |
| pH | 7.15 |
| Temperature | 37° C. |
| Reaction Time | 180 sec |
| Detection | |
| Wavelength | 405 nm |
| Measuring Time | 120 sec |

### Unit Definition

Bacillus stearothermophilus amylase (BS-amylase) activity is measured in KNU(S), Kilo Novo Units (sterarothermophilus), relative to an enzyme standard of a declared strength.

This analytical method is described in more details in EB-SM-0221.02 (incorporated by reference) available from Novozymes NS, Denmark, on request.

### Determination of FAU(F)

FAU(F) Fungal Alpha-Amylase Units (Fungamyl) is measured relative to an enzyme standard of a declared strength.

Reaction conditions

| | |
|---|---|
| Temperature | 37° C. |
| pH | 7.15 |
| Wavelength | 405 nm |
| Reaction time | 5 min |
| Measuring time | 2 min |

A folder (EB-SM-0216.02) describing this standard method in more detail is available on request from Novozymes A/S, Denmark, which folder is hereby included by reference.

### Determination of Pullulanase Activity (NPUN)

Endo-pullulanase activity in NPUN is measured relative to a Novozymes pullulanase standard. One pullulanase unit (NPUN) is defined as the amount of enzyme that releases 1 micro mol glucose per minute under the standard conditions (0.7% red pullulan (Megazyme), pH 5, 40° C., 20 minutes). The activity is measured in NPUN/ml using red pullulan.

1 mL diluted sample or standard is incubated at 40° C. for 2 minutes. 0.5 mL 2% red pullulan, 0.5 M KCl, 50 mM citric acid, pH 5 are added and mixed. The tubes are incubated at 40° C. for 20 minutes and stopped by adding 2.5 ml 80% ethanol. The tubes are left standing at room temperature for 10-60 minutes followed by centrifugation 10 minutes at 4000 rpm. OD of the supernatants is then measured at 510 nm and the activity calculated using a standard curve.

The present invention is described in further detail in the following examples which are offered to illustrate the present invention, but not in any way intended to limit the scope of the invention as claimed. All references cited herein are specifically incorporated by reference for that which is described therein.

### EXAMPLES

### Example 1

### Stability of Alpha-Amylase Variants

The stability of a reference alpha-amylase (Bacillus stearothermophilus alpha-amylase with the mutations I181*+G182*+N193F truncated to 491 amino acids (SEQ ID NO: 1 numbering)) and alpha-amylase variants thereof was determined by incubating the reference alpha-amylase and variants at pH 4.5 and 5.5 and temperatures of 75° C. and 85° C. with 0.12 mM CaCl₂ followed by residual activity determination using the EnzChek® substrate (EnzChek® Ultra Amylase assay kit, E33651, Molecular Probes).

Purified enzyme samples were diluted to working concentrations of 0.5 and 1 or 5 and 10 ppm (micrograms/ml) in enzyme dilution buffer (10 mM acetate, 0.01% Triton X100, 0.12 mM CaCl₂, pH 5.0). Twenty microliters enzyme sample was transferred to 48-well PCR MTP and 180 microliters stability buffer (150 mM acetate, 150 mM MES, 0.01% Triton X100, 0.12 mM CaCl₂, pH 4.5 or 5.5) was added to each well and mixed. The assay was performed using two concentrations of enzyme in duplicates. Before incubation at 75° C. or 85° C., 20 microliters was withdrawn and stored on ice as control samples. Incubation was performed in a PCR machine at 75° C. and 85° C. After incubation samples were diluted to 15 ng/mL in residual activity buffer (100 mM Acetate, 0.01% Triton X100, 0.12 mM CaCl₂, pH 5.5) and 25 microliters diluted enzyme was transferred to black 384-MTP. Residual activity was determined using the EnzChek substrate by adding 25 microliters substrate solution (100 micrograms/ml) to each well. Fluorescence was determined every minute for 15 minutes using excitation filter at 485-P nm and emission filter at 555 nm (fluorescence reader is Polarstar, BMG). The residual activity was normalized to control samples for each setup.

Assuming logarithmic decay half life time (T½ (min)) was calculated using the equation: T½(min)=T(min)*LN(0.5)/LN(% RA/100), where T is assay incubation time in minutes, and % RA is % residual activity determined in assay.

Using this assay setup the half life time was determined for the reference alpha-amylase and variant thereof as shown in Table 1.

TABLE 1

| | | T½ (min) | |
|---|---|---|---|
| | T½ (min) | (pH 4.5, 85° C., | T½ (min) |
| | (pH 4.5, 75° C., | 0.12 mM | (pH 5.5, 85° C., |
| Mutations | 0.12 mM CaCl₂) | CaCl₂) | 0.12 mM CaCl₂) |
| Reference Alpha-Amylase A | 21 | 4 | 111 |
| Reference Alpha-Amylase A with | 32 | 6 | 301 |
| the substitution V59A | | | |
| Reference Alpha-Amylase A with | 28 | 5 | 230 |
| the substitution V59E | | | |
| Reference Alpha-Amylase A with | 28 | 5 | 210 |
| the substitution V59I | | | |
| Reference Alpha-Amylase A with | 30 | 6 | 250 |
| the substitution V59Q | | | |
| Reference Alpha-Amylase A with | 149 | 22 | ND |
| the substitutions V59A + Q89R + | | | |
| G112D + E129V + K177L + R179E + | | | |
| K220P + N224L + Q254S | | | |
| Reference Alpha-Amylase A with | >180 | 28 | ND |
| the substitutions | | | |
| V59A + Q89R + E129V + | | | |
| K177L + R179E + H208Y + K220P + | | | |
| N224L + Q254S | | | |
| Reference Alpha-Amylase A with | 112 | 16 | ND |
| the substitutions | | | |
| V59A + Q89R + E129V + | | | |
| K177L + R179E + K220P + N224L + | | | |
| Q254S + D269E + D281N | | | |
| Reference Alpha-Amylase A with | 168 | 21 | ND |
| the substitutions | | | |
| V59A + Q89R + E129V + | | | |
| K177L + R179E + K220P + N224L + | | | |
| Q254S + I270L | | | |
| Reference Alpha-Amylase A with | >180 | 24 | ND |
| the substitutions | | | |
| V59A + Q89R + E129V + | | | |
| K177L + R179E + K220P + N224L + | | | |
| Q254S + H274K | | | |
| Reference Alpha-Amylase A with | 91 | 15 | ND |
| the substitutions | | | |
| V59A + Q89R + E129V + | | | |
| K177L + R179E + K220P + N224L + | | | |
| Q254S + Y276F | | | |
| Reference Alpha-Amylase A with | 141 | 41 | ND |
| the substitutions V59A + E129V + | | | |
| R157Y + K177L + R179E + K220P + | | | |
| N224L + S242Q + Q254S | | | |
| Reference Alpha-Amylase A with | >180 | 62 | ND |
| the substitutions V59A + E129V + | | | |
| K177L + R179E + H208Y + K220P + | | | |
| N224L + S242Q + Q254S | | | |
| Reference Alpha-Amylase A with | >180 | 49 | >480 |
| the substitutions V59A + E129V + | | | |
| K177L + R179E + K220P + N224L + | | | |
| S242Q + Q254S | | | |
| Reference Alpha-Amylase A with | >180 | 53 | ND |
| the substitutions V59A + E129V + | | | |
| K177L + R179E + K220P + N224L + | | | |
| S242Q + Q254S + H274K | | | |
| Reference Alpha-Amylase A with | >180 | 57 | ND |
| the substitutions V59A + E129V + | | | |
| K177L + R179E + K220P + N224L + | | | |
| S242Q + Q254S + Y276F | | | |
| Reference Alpha-Amylase A with | >180 | 37 | ND |
| the substitutions V59A + E129V + | | | |
| K177L + R179E + K220P + N224L + | | | |
| S242Q + Q254S + D281N | | | |
| Reference Alpha-Amylase A with | >180 | 51 | ND |
| the substitutions V59A + E129V + | | | |
| K177L + R179E + K220P + N224L + | | | |
| S242Q + Q254S + M284T | | | |
| Reference Alpha-Amylase A with | >180 | 45 | ND |
| the substitutions V59A + E129V + | | | |
| K177L + R179E + K220P + N224L + | | | |
| S242Q + Q254S + G416V | | | |
| Reference Alpha-Amylase A with | 143 | 21 | >480 |
| the substitutions V59A + E129V + | | | |
| K177L + R179E + K220P + N224L + | | | |
| Q254S | | | |
| Reference Alpha-Amylase A with | >180 | 22 | ND |
| the substitutions V59A + E129V + | | | |
| K177L + R179E + K220P + N224L + | | | |
| Q254S + M284T | | | |
| Reference Alpha-Amylase A with | >180 | 38 | ND |
| the substitutions | | | |
| A91L + M96I + E129V + | | | |
| K177L + R179E + K220P + N224L + | | | |
| S242Q + Q254S | | | |
| Reference Alpha-Amylase A with | 57 | 11 | 402 |
| the substitutions E129V + K177L + | | | |
| R179E | | | |
| Reference Alpha-Amylase A with | 174 | 44 | >480 |
| the substitutions E129V + K177L + | | | |
| R179E + K220P + N224L + S242Q + | | | |
| Q254S | | | |
| Reference Alpha-Amylase A with | >180 | 49 | >480 |
| the substitutions E129V + K177L + | | | |
| R179E + K220P + N224L + S242Q + | | | |
| Q254S + Y276F + L427M | | | |
| Reference Alpha-Amylase A with | >180 | 49 | >480 |
| the substitutions E129V + K177L + | | | |
| R179E + K220P + N224L + S242Q + | | | |
| Q254S + M284T | | | |
| Reference Alpha-Amylase A with | 177 | 36 | >480 |
| the substitutions E129V + K177L + | | | |
| R179E + K220P + N224L + S242Q + | | | |
| Q254S + N376* + I377* | | | |
| Reference Alpha-Amylase A with | 94 | 13 | >480 |
| the substitutions E129V + K177L + | | | |
| R179E + K220P + N224L + Q254S | | | |
| Reference Alpha-Amylase A with | 129 | 24 | >480 |
| the substitutions E129V + K177L + | | | |
| R179E + K220P + N224L + Q254S + | | | |
| M284T | | | |
| Reference Alpha-Amylase A with | 148 | 30 | >480 |
| the substitutions E129V + K177L + | | | |
| R179E + S242Q | | | |
| Reference Alpha-Amylase A with | 78 | 9 | >480 |
| the substitutions E129V + K177L + | | | |
| R179V | | | |
| Reference Alpha-Amylase A with | 178 | 31 | >480 |
| the substitutions E129V + K177L + | | | |
| R179V + K220P + N224L + S242Q + | | | |
| Q254S | | | |
| Reference Alpha-Amylase A with | 66 | 17 | >480 |
| the substitutions K220P + N224L + | | | |
| S242Q + Q254S | | | |
| Reference Alpha-Amylase A with | 30 | 6 | 159 |
| the substitutions K220P + N224L + | | | |
| Q254S | | | |
| Reference Alpha-Amylase A with | 35 | 7 | 278 |
| the substitution M284T | | | |
| Reference Alpha-Amylase A with | 59 | 13 | ND |
| the substitutions M284V | | | |

ND not determined

The results demonstrate that the alpha-amylase variants have a significantly greater half-life and stability than the reference alpha-amylase.

### Example 2

### Preparation of Protease Variants and Test of Thermostability

### Strains and Plasmids

E. coli DH12S (available from Gibco BRL) was used for yeast plasmid rescue. pJTP000 is a S. cerevisiae and E. coli shuttle vector under the control of TPI promoter, constructed from pJC039 described in WO 01/92502, in which the Thermoascus aurantiacus M35 protease gene (WO 03048353) has been inserted.

Saccharomyces cerevisiae YNG318 competent cells: MATa Dpep4[cir+] ura3-52, leu2-D2, his 4-539 was used for protease variants expression. It is described in J. Biol. Chem. 272 (15), pp 9720-9727, 1997.

### Media and Substrates

10× Basal solution: Yeast nitrogen base w/o amino acids (DIFCO) 66.8 g/l, succinate 100 g/l, NaOH 60 g/l.
SC-glucose: 20% glucose (i.e., a final concentration of 2%=2 g/100 ml)) 100 ml/1, 5% threonine 4 ml/1, 1% tryptophan 10 ml/l, 20% casamino acids 25 ml/1, 10× basal solution 100 ml/l. The solution is sterilized using a filter of a pore size of 0.20 micrometer. Agar (2%) and H₂O (approx. 761 ml) is autoclaved together, and the separately sterilized SC-glucose solution is added to the agar solution.
YPD: Bacto peptone 20 g/l, yeast extract 10 g/l, 20% glucose 100 ml/1.

### YPD+Zn: YPD+0.25 mM ZnSO₄.

PEG/LiAc solution: 40% PEG4000 50 ml, 5 M Lithium Acetate 1 ml.
96 well Zein micro titre plate:

Each well contains 200 microL of 0.05-0.1% of zein (Sigma), 0.25 mM ZnSO₄ and 1% of agar in 20 mM sodium acetate buffer, pH 4.5.

### DNA Manipulations

Unless otherwise stated, DNA manipulations and transformations were performed using standard methods of molecular biology as described in Sambrook et al. (1989) Molecular cloning: A laboratory manual, Cold Spring Harbor lab. Cold Spring Harbor, N.Y.; Ausubel, F. M. et al. (eds.) “Current protocols in Molecular Biology”, John Wiley and Sons, 1995; Harwood, C. R. and Cutting, S. M. (Eds.).

### Yeast Transformation

Yeast transformation was performed using the lithium acetate method. 0.5 microL of vector (digested by restriction endonucleases) and 1 microL of PCR fragments is mixed. The DNA mixture, 100 microL of YNG318 competent cells, and 10 microL of YEAST MAKER carrier DNA (Clontech) is added to a 12 ml polypropylene tube (Falcon 2059). Add 0.6 ml PEG/LiAc solution and mix gently. Incubate for 30 min at 30° C., and 200 rpm followed by 30 min at 42° C. (heat shock). Transfer to an eppendorf tube and centrifuge for 5 sec. Remove the supernatant and resolve in 3 ml of YPD. Incubate the cell suspension for 45 min at 200 rpm at 30° C. Pour the suspension to SC-glucose plates and incubate 30° C. for 3 days to grow colonies. Yeast total DNA are extracted by Zymoprep Yeast Plasmid Miniprep Kit (ZYMO research).

### DNA Sequencing

E. coli transformation for DNA sequencing was carried out by electroporation (BIO-RAD Gene Pulser). DNA Plasmids were prepared by alkaline method (Molecular Cloning, Cold Spring Harbor) or with the Qiagen® Plasmid Kit. DNA fragments were recovered from agarose gel by the Qiagen gel extraction Kit. PCR was performed using a PTC-200 DNA Engine. The ABI PRISM™ 310 Genetic Analyzer was used for determination of all DNA sequences.

### Construction of Protease Expression Vector

The Thermoascus M35 protease gene was amplified with the primer pair Prot F (SEQ ID NO: 4) and Prot R (SEQ ID NO: 5). The resulting PCR fragments were introduced into S. cerevisiae YNG318 together with the pJC039 vector (described in WO 2001/92502) digested with restriction enzymes to remove the Humicola insolens cutinase gene.

The Plasmid in yeast clones on SC-glucose plates was recovered to confirm the internal sequence and termed as pJTP001.

### Construction of Yeast Library and Site-Directed Variants

Library in yeast and site-directed variants were constructed by SOE PCR method (Splicing by Overlap Extension, see “PCR: A practical approach”, p. 207-209, Oxford University press, eds. McPherson, Quirke, Taylor), followed by yeast in vivo recombination.

### General Primers for Amplification and Sequencing

The primers AM34 (SEQ ID NO: 6) and AM35 (SEQ ID NO:7) were used to make DNA fragments containing any mutated fragments by the SOE method together with degenerated primers (AM34+Reverse primer and AM35+forward primer) or just to amplify a whole protease gene (AM34+AM35).

| PCR reaction system: | Conditions: | |
|---|---|---|
| 48.5 microL H₂O | 1 | 94° C. 2 min |
| 2 beads puRe Taq Ready-To-Go PCR | 2 | 94° C. 30 sec |
| (Amersham Biosciences) | | |
| 0.5 micro L X 2 100 pmole/microL of primers | 3 | 55° C. 30 sec |
| 0.5 microL template DNA | 4 | 72° C. 90 sec |
| | 2-4 | 25 cycles |
| | 5 | 72° C. 10 min |

DNA fragments were recovered from agarose gel by the Qiagen gel extraction Kit. The resulting purified fragments were mixed with the vector digest. The mixed solution was introduced into Saccharomyces cerevisiae to construct libraries or site-directed variants by in vivo recombination.

### Relative Activity Assay

Yeast clones on SC-glucose were inoculated to a well of a 96-well micro titre plate containing YPD+Zn medium and cultivated at 28° C. for 3 days. The culture supernatants were applied to a 96-well zein micro titer plate and incubated at at least 2 temperatures (ex. 60° C. and 65° C., 70° C. and 75° C., 70° C. and 80° C.) for more than 4 hours or overnight. The turbidity of zein in the plate was measured as A630 and the relative activity (higher/lower temperatures) was determined as an indicator of thermoactivity improvement. The clones with higher relative activity than the parental variant were selected and the sequence was determined.

### Remaining Activity Assay

Yeast clones on SC-glucose were inoculated to a well of a 96-well micro titre plate and cultivated at 28° C. for 3 days. Protease activity was measured at 65° C. using azo-casein (Megazyme) after incubating the culture supernatant in 20 mM sodium acetate buffer, pH 4.5, for 10 min at a certain temperature (80° C. or 84° C. with 4° C. as a reference) to determine the remaining activity. The clones with higher remaining activity than the parental variant were selected and the sequence was determined.

### Azo-Casein Assay

20 microL of samples were mixed with 150 microL of substrate solution (4 ml of 12.5% azo-casein in ethanol in 96 ml of 20 mM sodium acetate, pH 4.5, containing 0.01% triton-100 and 0.25 mM ZnSO₄) and incubated for 4 hours or longer.

After adding 20 microL/well of 100% trichloroacetic acid (TCA) solution, the plate was centrifuge and 100 microL of supernatants were pipette out to measure A440.

Expression of Protease Variants in Aspergillus oryzae

The constructs comprising the protease variant genes were used to construct expression vectors for Aspergillus. The Aspergillus expression vectors consist of an expression cassette based on the Aspergillus niger neutral amylase II promoter fused to the Aspergillus nidulans triose phosphate isomerase non translated leader sequence (Pna2/tpi) and the Aspergillus niger amyloglucosidase terminator (Tamg). Also present on the plasmid was the Aspergillus selective marker amdS from Aspergillus nidulans enabling growth on acetamide as sole nitrogen source. The expression plasmids for protease variants were transformed into Aspergillus as described in Lassen et al. (2001), Appl. Environ. Microbiol. 67, 4701-4707. For each of the constructs 10-20 strains were isolated, purified and cultivated in shake flasks.

### Purification of Expressed Variants

1. Adjust pH of the 0.22 μm filtered fermentation sample to 4.0.
2. Put the sample on an ice bath with magnetic stirring. Add (NH4)2SO4 in small aliquots (corresponding to approx. 2.0-2.2 M (NH4)2SO4 not taking the volume increase into account when adding the compound).
3. After the final addition of (NH4)2SO4, incubate the sample on the ice bath with gentle magnetic stirring for min. 45 min.
4. Centrifugation: Hitachi himac CR20G High-Speed Refrigerated Centrifuge equipped with R20A2 rotor head, 5° C., 20,000 rpm, 30 min.
5. Dissolve the formed precipitate in 200 ml 50 mM Na-acetate pH 4.0.
6. Filter the sample by vacuum suction using a 0.22 μm PES PLUS membrane (IWAKI).
7. Desalt/buffer-exchange the sample to 50 mM Na-acetate pH 4.0 using ultrafiltration (Vivacell 250 from Vivascience equipped with 5 kDa MWCO PES membrane) overnight in a cold room. Dilute the retentate sample to 200 ml using 50 mM Na-acetate pH 4.0. The conductivity of sample is preferably less than 5 mS/cm.
8. Load the sample onto a cation-exchange column equilibrated with 50 mM Na-acetate pH 4.0. Wash unbound sample out of the column using 3 column volumes of binding buffer (50 mM Na-acetate pH 4.0), and elute the sample using a linear gradient, 0-100% elution buffer (50 mM Na-acetate+1 M NaCl pH 4.0) in 10 column volumes.
9. The collected fractions are assayed by an endo-protease assay (cf. below) followed by standard SDS-PAGE (reducing conditions) on selected fractions. Fractions are pooled based on the endo-protease assay and SDS-PAGE.

### Endo-Protease Assay

1. Protazyme OL tablet/5 ml 250 mM Na-acetate pH 5.0 is dissolved by magnetic stirring (substrate: endo-protease Protazyme AK tablet from Megazyme—cat. # PRAK 11/08).
2. With stirring, 250 microL of substrate solution is transferred to a 1.5 ml Eppendorf tube.
3. 25 microL of sample is added to each tube (blank is sample buffer).
4. The tubes are incubated on a Thermomixer with shaking (1000 rpm) at 50° C. for 15 minutes.
5. 250 microL of 1 M NaOH is added to each tube, followed by vortexing.
6. Centrifugation for 3 min. at 16,100×G and 25° C.
7. 200 microL of the supernatant is transferred to a MTP, and the absorbance at 590 nm is recorded.

### Results

TABLE 2
Relative activity of protease variants. Numbering of substitution(s)
starts from N-terminal of the mature peptide in amino acids 1 to 177 of
SEQ ID NO: 3.

| | | Relative activity |
|---|---|---|
| Variant | Substitution(s) | 65° C./60° C. |
| WT | none | 31% |
| JTP004 | S87P | 45% |
| JTP005 | A112P | 43% |
| JTP008 | R2P | 71% |
| JTP009 | D79K | 69% |
| JTP010 | D79L | 75% |
| JTP011 | D79M | 73% |
| JTP012 | D79L/S87P | 86% |
| JTP013 | D79L/S87P/A112P | 90% |
| JTP014 | D79L/S87P/A112P | 88% |
| JTP016 | A73C | 52% |
| JTP019 | A126V | 69% |
| JTP021 | M152R | 59% |

TABLE 3
Relative activity of protease variants. Numbering of substitution(s)
starts from N-terminal of the mature peptide in amino acids
1 to 177 of SEQ ID NO: 3.

| | | Relative activity | | |
|---|---|---|---|---|
| | | 70° C./ | 75° C./ | 75° C./ |
| Variant | Substitution(s) and/or deletion (S) | 65° C. | 65° C. | 70° C. |
| WT | none | 59% | 17% | |
| JTP036 | D79L/S87P/D142L | 73% | 73% | |
| JTP040 | T54R/D79L/S87P | | 71% | |
| JTP042 | Q53K/D79L/S87P/I173V | | 108% | |
| JTP043 | Q53R/D79L/S87P | | 80% | |
| JTP045 | S41R/D79L/S87P | | 82% | |
| JTP046 | D79L/S87P/Q158W | | 96% | |
| JTP047 | D79L/S87P/S157K | | 85% | |
| JTP048 | D79L/S87P/D104R | | 88% | |
| JTP050 | D79L/S87P/A112P/D142L | | 88% | |
| JTP051 | S41R/D79L/S87P/A112P/D142L | | | 102% |
| JTP052 | D79L/S87P/A112P/D142L/S157K | | | 111% |
| JTP053 | S41R/D79L/S87P/A112P/D142L/ | | | 113% |
| | S157K | | | |
| JTP054 | ΔS5/D79L/S87P | | | 92% |
| JTP055 | ΔG8/D79L/S87P | | | 95% |
| JTP059 | C6R/D79L/S87P | | | 92% |
| JTP061 | T46R/D79L/S87P | | | 111% |
| JTP063 | S49R/D79L/S87P | | | 94% |
| JTP064 | D79L/S87P/N88R | | | 92% |
| JTP068 | D79L/S87P/T114P | | | 99% |
| JTP069 | D79L/S87P/S115R | | | 103% |
| JTP071 | D79L/S87P/T116V | | | 105% |
| JTP072 | N26R/D79L/S87P | | 92% | |
| JTP077 | A27K/D79L/S87P/A112P/D142L | | 106% | |
| JTP078 | A27V/D79L/S87P/A112P/D142L | | 100% | |
| JTP079 | A27G/D79L/S87P/A112P/D142L | | 104% | |

TABLE 4
Relative activity of protease variants. Numbering of substitution(s) starts from
N-terminal of the mature peptide in amino acids 1 to 177 of SEQ ID NO: 3.

| | | Relative | Remaining | |
|---|---|---|---|---|
| | | activity | activity | |
| Variant | Substitution(s) and/or deletion(s) | 75° C./65° C. | 80° C. | 84° C. |
| JTP082 | ΔS5/D79L/S87P/A112P/D142L | 129% | | 53% |
| JTP083 | T46R/D79L/S87P/A112P/D142L | 126% | | |
| JTP088 | Y43F/D79L/S87P/A112P/D142L | 119% | | |
| JTP090 | D79L/S87P/A112P/T124L/D142L | 141% | | |
| JTP091 | D79L/S87P/A112P/T124V/D142L | 154% | 43% | |
| JTP092 | ΔS5/N26R/D79L/S87P/A112P/D142L | | | 60% |
| JTP095 | N26R/T46R/D79L/S87P/A112P/D142L | | | 62% |
| JTP096 | T46R/D79L/S87P/T116V/D142L | | | 67% |
| JTP099 | D79L/P81R/S87P/A112P/D142L | | | 80% |
| JTP101 | A27K/D79L/S87P/A112P/T124V/D142L | | 81% | |
| JTP116 | D79L/Y82F/S87P/A112P/T124V/D142L | | 59% | |
| JTP117 | D79L/Y82F/S87P/A112P/T124V/D142L | | 94% | |
| JTP127 | D79L/S87P/A112P/T124V/A126V/D142L | | 53% | |

TABLE 5
Relative activity of protease variants. Numbering of substitution(s) starts from
N-terminal of the mature peptide in amino acids 1 to 177 of SEQ ID NO: 3.

| | | Relative activity | | |
|---|---|---|---|---|
| Variant | Substitutions | 75° C./70° C. | 80° C./70° C. | 85° C./70° C. |
| JTP050 | D79L S87P A112P D142L | 55% | 23% | 9% |
| JTP134 | D79L Y82F S87P A112P D142L | | 40% | |
| JTP135 | S38T D79L S87P A112P A126V D142L | | 62% | |
| JTP136 | D79L Y82F S87P A112P A126V D142L | | 59% | |
| JTP137 | A27K D79L S87P A112P A126V D142L | | 54% | |
| JTP140 | D79L S87P N98C A112P G135C D142L | 81% | | |
| JTP141 | D79L S87P A112P D142L T141C M161C | 68% | | |
| JTP143 | S36P D79L S87P A112P D142L | 69% | | |
| JTP144 | A37P D79L S87P A112P D142L | 57% | | |
| JTP145 | S49P D79L S87P A112P D142L | 82% | 59% | |
| JTP146 | S50P D79L S87P A112P D142L | 83% | 63% | |
| JTP148 | D79L S87P D104P A112P D142L | 76% | 64% | |
| JTP161 | D79L Y82F S87G A112P D142L | | 30% | 12% |
| JTP180 | S70V D79L Y82F S87G Y97W A112P | | 52% | |
| | D142L | | | |
| JTP181 | D79L Y82F S87G Y97W D104P A112P | | 45% | |
| | D142L | | | |
| JTP187 | S70V D79L Y82F S87G A112P D142L | | 45% | |
| JTP188 | D79L Y82F S87G D104P A112P D142L | | 43% | |
| JTP189 | D79L Y82F S87G A112P A126V D142L | | 46% | |
| JTP193 | Y82F S87G S70V D79L D104P A112P | | | 15% |
| | D142L | | | |
| JTP194 | Y82F S87G D79L D104P A112P A126V | | | 22% |
| | D142L | | | |
| JTP196 | A27K D79L Y82F S87G D104P A112P | | | 18% |
| | A126V D142L | | | |

Relative activity

| Variant | Substitutions | 75° C./70° C. | 80° C./70° C. |
|---|---|---|---|
| JTP196 | A27K D79L Y82F | 102% | 55% |
| | S87G D104P A112P | | |
| | A126V D142L | | |
| JTP210 | A27K Y82F S87G | 107% | 36% |
| | D104P A112P A126V | | |
| | D142L | | |
| JTP211 | A27K D79L Y82F | 94% | 44% |
| | D104P A112P A126V | | |
| | D142L | | |
| JTP213 | A27K Y82F D104P | 103% | 37% |
| | A112P A126V D142L | | |

### Example 3

### Temperature Profile of Selected Variants Using Purified Enzymes

Selected variants showing good thermo-stability were purified and the purified enzymes were used in a zein-BCA assay as described below. The remaining protease activity was determined at 60° C. after incubation of the enzyme at elevated temperatures as indicated for 60 min.

Zein-BCA assay:

Zein-BCA assay was performed to detect soluble protein quantification released from zein by variant proteases at various temperatures.

### Protocol:

1) Mix 10 ul of 10 ug/ml enzyme solutions and 100 ul of 0.025% zein solution in a micro titer plate (MTP).
2) Incubate at various temperatures for 60 min.
3) Add 10 ul of 100% trichloroacetic acid (TCA) solution.
4) Centrifuge MTP at 3500 rpm for 5 min.
5) Take out 15 ul to a new MTP containing 100 ul of BCA assay solution (Pierce Cat#:23225, BCA Protein Assay Kit).
6) Incubate for 30 min. at 60° C.
7) Measure A562.

The results are shown in Table 6. All of the tested variants showed an improved thermo-stability as compared to the wt protease.

TABLE 6
Zein-BCA assay

| | Sample incubated 60 min at indicated temperatures (° C.) | | | | | | |
|---|---|---|---|---|---|---|---|
| | (μg/ml Bovine serum albumin equivalent peptide released) | | | | | | |
| WT/ | | | | | | | 95° |
| Variant | 60° C. | 70° C. | 75° C. | 80° C. | 85° C. | 90° C. | C. |
| WT | 94 | 103 | 107 | 93 | 58 | 38 | |
| JTP050 | 86 | 101 | 107 | 107 | 104 | 63 | 36 |
| JTP077 | 82 | 94 | 104 | 105 | 99 | 56 | 31 |
| JTP188 | 71 | 83 | 86 | 93 | 100 | 75 | 53 |
| JTP196 | 87 | 99 | 103 | 106 | 117 | 90 | 38 |

### Example 4

Characterization of Penicillium oxalicum Glucoamylase

The Penicillium oxalicum glucoamylase is disclosed in SEQ ID NO: 9 herein.

### Substrate.

Substrate: 1% soluble starch (Sigma S-9765) in deionized water

Reaction buffer: 0.1 M Acetate buffer at pH 5.3
Glucose concentration determination kit: Wako glucose assay kit (LabAssay glucose, WAKO, Cat#298-65701).

### Reaction Condition.

20 microL soluble starch and 50 microL acetate buffer at pH 5.3 were mixed. 30 microL enzyme solution (50 micro g enzyme protein/ml) was added to a final volume of 100 microL followed by incubation at 37° C. for 15 min.

The glucose concentration was determined by Wako kits.

All the work carried out in parallel.

### Temperature Optimum.

To assess the temperature optimum of the Penicillium oxalicum glucoamylase the “Reaction condition”-assay described above was performed at 20, 30, 40, 50, 60, 70, 80, 85, 90 and 95° C. The results are shown in Table 7.

TABLE 7
Temperature optimum

| | Temperature (° C.) | | | | | | | | | |
|---|---|---|---|---|---|---|---|---|---|---|
| | 20 | 30 | 40 | 50 | 60 | 70 | 80 | 85 | 90 | 95 |
| Relative activity | 63.6 | 71.7 | 86.4 | 99.4 | 94.6 | 100.0 | 92.9 | 92.5 | 82.7 | 82.8 |
| (%) | | | | | | | | | | |

From the results it can be seen that the optimal temperature for Penicillium oxalicum glucoamylase at the given conditions is between 50° C. and 70° C. and the glucoamylase maintains more than 80% activity at 95° C.

### Heat Stability.

To assess the heat stability of the Penicillium oxalicum glucoamylase the Reaction condition assay was modifed in that the the enzyme solution and acetate buffer was preincubated for 15 min at 20, 30, 40, 50, 60, 70, 75, 80, 85, 90 and 95° C. Following the incubation 20 microL of starch was added to the solution and the assay was performed as described above.

The results are shown in Table 8.

TABLE 8
Heat stability

| | Temperature (° C.) | | | | | | | | | |
|---|---|---|---|---|---|---|---|---|---|---|
| | 20 | 30 | 40 | 50 | 60 | 70 | 80 | 85 | 90 | 95 |
| Relative activity | 91.0 | 92.9 | 88.1 | 100.0 | 96.9 | 86.0 | 34.8 | 36.0 | 34.2 | 34.8 |
| (%) | | | | | | | | | | |

From the results it can be seen that Penicillium oxalicum glucoamylase is stable up to 70° C. after preincubation for 15 min in that it maintains more than 80% activity.

pH optimum. To assess the pH optimum of the Penicillium oxalicum glucoamylase the Reaction condition assay described above was performed at pH 2.0, 3.0, 3.5, 4.0, 4.5, 5.0, 6.0 7.0, 8.0, 9.0, 10.0 and 11.0. Instead of using the acetate buffer described in the Reaction condition assay the following buffer was used 100 mM Succinic acid, HEPES, CHES, CAPSO, 1 mM CaCl₂, 150 mM KCl, 0.01% Triton X-100, pH adjusted to 2.0, 3.0, 3.5, 4.0, 4.5, 5.0, 6.0 7.0, 8.0, 9.0, 10.0 or 11.0 with HCl or NaOH.

The results are shown in Table 9.

TABLE 9
pH optimum

| | pH | | | | | | | | | | | |
|---|---|---|---|---|---|---|---|---|---|---|---|---|
| | 2.0 | 3.0 | 3.5 | 4.0 | 4.5 | 5.0 | 6.0 | 7.0 | 8.0 | 9.0 | 10.0 | 11.0 |
| Relative | 71.4 | 78.6 | 77.0 | 91.2 | 84.2 | 100.0 | 55.5 | 66.7 | 30.9 | 17.8 | 15.9 | 16.1 |
| activity | | | | | | | | | | | | |
| (%) | | | | | | | | | | | | |

From the results it can be seen that Penicillium oxalicum glucoamylase at the given conditions has the highest activity at pH 5.0. The Penicillium oxalicum glucoamylase is active in a broad pH range in the it maintains more than 50% activity from pH 2 to 7.

### pH Stability.

To assess the heat stability of the Penicillium oxalicum glucoamylase the Reaction condition assay was modifed in that the enzyme solution (50 micro g/mL) was preincubated for 20 hours in buffers with pH 2.0, 3.0, 3.5, 4.0, 4.5, 5.0, 6.0 7.0, 8.0, 9.0, 10.0 and 11.0 using the buffers described under pH optimum. After preincubation, 20 microL soluble starch to a final volume of 100 microL was added to the solution and the assay was performed as described above.

The results are shown in Table 10.

TABLE 10
pH stability

| | pH | | | | | | | | | | | |
|---|---|---|---|---|---|---|---|---|---|---|---|---|
| | 2.0 | 3.0 | 3.5 | 4.0 | 4.5 | 5.0 | 6.0 | 7.0 | 8.0 | 9.0 | 10.0 | 11.0 |
| Relative | 17.4 | 98.0 | 98.0 | 103.2 | 100.0 | 93.4 | 71.2 | 90.7 | 58.7 | 17.4 | 17.0 | 17.2 |
| activity | | | | | | | | | | | | |
| (%) | | | | | | | | | | | | |

From the results it can be seen that Penicillium oxalicum glucoamylase, is stable from pH 3 to pH 7 after preincubation for 20 hours and it decreases its activity at pH 8.

### Example 5

### Thermostability of Protease Pfu.

The thermostability of the Pyrococcus furiosus protease (Pfu S) purchased from Takara Bio Inc, (Japan) was tested using the same methods as in Example 2. It was found that the thermostability (Relative Activity) was 110% at (80° C./70° C.) and 103% (90° C./70° C.) at pH 4.5.

### Example 6

Cloning of Penicillium oxalicum Strain Glucoamylase Gene
Preparation of Penicillium oxalicum Strain cDNA.

The cDNA was synthesized by following the instruction of 3′ Rapid Amplifiction of cDNA End System (Invitrogen Corp., Carlsbad, Calif., USA).

Cloning of Penicillium oxalicum Strain Glucoamylase Gene.

The Penicillium oxalicum glucoamylase gene was cloned using the oligonucleotide primer shown below designed to amplify the glucoamylase gene from 5′ end.

Sense primer:
(SEQ ID NO: 22)
5′-ATGCGTCTCACTCTATTATCAGGTG-3′

The full length gene was amplified by PCR with Sense primer and AUAP (supplied by 3′ Rapid Amplifiction of cDNA End System) by using Platinum HIFI Taq DNA polymerase (Invitrogen Corp., Carlsbad, Calif., USA). The amplification reaction was composed of 5 μl of 10×PCR buffer, 2 μl of 25 mM MgCl₂, 1 μl of 10 mM dNTP, 1 μl of 10 uM Sense primer, 1 μl of 10 uM AUAP, 2 μl of the first strand cDNA, 0.5 μl of HIFI Taq, and 37.5 μl of deionized water. The PCR program was: 94° C., 3 mins; 10 cycles of 94° C. for 40 secs, 60° C. 40 secs with 1° C. decrease per cycle, 68° C. for 2 min; 25 cycles of 94° C. for 40 secs, 50° C. for 40 secs, 68° C. for 2 min; final extension at 68° C. for 10 mins.

The obtained PCR fragment was cloned into pGEM-T vector (Promega Corporation, Madison, Wis., USA) using a pGEM-T Vector System (Promega Corporation, Madison, Wis., USA) to generate plasmid AMG 1. The glucoamylase gene inserted in the plasmid AMG 1 was sequencing confirmed. E. coli strain TOP10 containing plasmid AMG 1 (designated NN059173), was deposited with the Deutsche Sammlung von Mikroorganismen and Zellkulturen GmbH (DSMZ) on Nov. 23, 2009, and assigned accession number as DSM 23123.

### Example 7

Expression of Cloned Penicillium oxalicum Glucoamylase

The Penicillium oxalicum glucoamylase gene was re-cloned from the plasmid AMG 1 into an Aspergillus expression vector by PCR using two cloning primer F and primer R shown below, which were designed based on the known sequence and added tags for direct cloning by IN-FUSION™ strategy.

Primer F:
(SEQ ID NO: 23)
5′ ACACAACTGGGGATCCACCATGCGTCTCACTCTATTATC
Primer R:
(SEQ ID NO: 24)
5′ AGATCTCGAGAAGCTTAAAACTGCCACACGTCGTTGG

A PCR reaction was performed with plasmid AMG 1 in order to amplify the full-length gene. The PCR reaction was composed of 40 μg of the plasmid AMG 1 DNA, 1 μl of each primer (100 μM); 12.5 μl of 2×Extensor Hi-Fidelity master mix (Extensor Hi-Fidelity Master Mix, ABgene, United Kingdom), and 9.5 μl of PCR-grade water. The PCR reaction was performed using a DYAD PCR machine (Bio-Rad Laboratories, Inc., Hercules, Calif., USA) programmed for 2 minutes at 94° C. followed by a 25 cycles of 94° C. for 15 seconds, 50° C. for 30 seconds, and 72° C. for 1 minute; and then 10 minutes at 72° C.

The reaction products were isolated by 1.0% agarose gel electrophoresis using 1×TAE buffer where an approximately 1.9 kb PCR product band was excised from the gel and purified using a GFX® PCR DNA and Gel Band Purification Kit (GE Healthcare, United Kingdom) according to manufacturer's instructions. DNA corresponding to the Penicillium oxalicum glucoamylase gene was cloned into an Aspergillus expression vector linearized with BamHI and HindIII, using an IN-FUSION™ Dry-Down PCR Cloning Kit (BD Biosciences, Palo Alto, Calif., USA) according to the manufacturer's instructions. The linearized vector construction is as described in WO 2005/042735 A1.

A 2 μl volume of the ligation mixture was used to transform 25 μl of Fusion Blue E. coli cells (included in the IN-FUSION™ Dry-Down PCR Cloning Kit). After a heat shock at 42° C. for 45 sec, and chilling on ice, 250 μl of SOC medium was added, and the cells were incubated at 37° C. at 225 rpm for 90 min before being plated out on LB agar plates containing 50 μg of ampicillin per ml, and cultivated overnight at 37° C. Selected colonies were inoculated in 3 ml of LB medium supplemented with 50 μg of ampicillin per ml and incubated at 37° C. at 225 rpm overnight. Plasmid DNA from the selected colonies was purified using Mini JETSTAR (Genomed, Germany) according to the manufacturer's instructions. Penicillium oxalicum glucoamylase gene sequence was verified by Sanger sequencing before heterologous expression. One of the plasmids was selected for further expression, and was named XYZ XYZ1471-4.

Protoplasts of Aspergillus niger MBin118 were prepared as described in WO 95/02043. One hundred μl of protoplast suspension were mixed with 2.5 μg of the XYZ1471-4 plasmid and 250 microliters of 60% PEG 4000 (Applichem) (polyethylene glycol, molecular weight 4,000), 10 mM CaCl₂, and 10 mM Tris-HCl pH 7.5 were added and gently mixed. The mixture was incubated at 37° C. for 30 minutes and the protoplasts were mixed with 6% low melting agarose (Biowhittaker Molecular Applications) in COVE sucrose (Cove, 1996, Biochim. Biophys. Acta 133:51-56) (1M) plates supplemented with 10 mM acetamide and 15 mM CsCl and added as a top layer on COVE sucrose (1M) plates supplemented with 10 mM acetamide and 15 mM CsCl for transformants selection (4 ml topagar per plate). After incubation for 5 days at 37° C. spores of sixteen transformants were picked up and seed on 750 μl YP-2% Maltose medium in 96 deepwell MT plates. After 5 days of stationary cultivation at 30° C., 10 μl of the culture-broth from each well was analyzed on a SDS-PAGE (Sodium dodecyl sulfate-polyacrylamide gel electrophoresis) gel, Griton XT Precast gel (BioRad, CA, USA) in order to identify the best transformants based on the ability to produce large amount of glucoamylase. A selected transformant was identified on the original transformation plate and was preserved as spores in a 20% glycerol stock and stored frozen (−80° C.).

Cultivation.

The selected transformant was inoculated in 100 ml of MLC media and cultivated at 30° C. for 2 days in 500 ml shake flasks on a rotary shaker. 3 ml of the culture broth was inoculated to 100 ml of M410 medium and cultivated at 30° C. for 3 days. The culture broth was centrifugated and the supernatant was filtrated using 0.2 μm membrane filters.

Alpha-Cyclodextrin Affinity Gel.

Ten grams of Epoxy-activated Sepharose 6B (GE Healthcare, Chalfont St. Giles, U.K) powder was suspended in and washed with distilled water on a sintered glass filter. The gel was suspended in coupling solution (100 ml of 12.5 mg/ml alpha-cyclodextrin, 0.5 M NaOH) and incubated at room temperature for one day with gentle shaking. The gel was washed with distilled water on a sintered glass filter, suspended in 100 ml of 1 M ethanolamine, pH 10, and incubated at 50° C. for 4 hours for blocking. The gel was then washed several times using 50 mM Tris-HCl, pH 8 and 50 mM NaOAc, pH 4.0 alternatively. The gel was finally packed in a 35-40 ml column using equilibration buffer (50 mM NaOAc, 150 mM NaCl, pH 4.5).

Purification of Glucoamylase from Culture Broth.

Culture broth from fermentation of A. niger MBin118 harboring the glucoamylase gene was filtrated through a 0.22 μm PES filter, and applied on a alpha-cyclodextrin affinity gel column previously equilibrated in 50 mM NaOAc, 150 mM NaCl, pH 4.5 buffer. Unbound material was washed off the column with equilibration buffer and the glucoamylase was eluted using the same buffer containing 10 mM beta-cyclodextrin over 3 column volumes.

The glucoamylase activity of the eluent was checked to see, if the glucoamylase had bound to the alpha-cyclodextrin affinity gel. The purified glucoamylase sample was then dialyzed against 20 mM NaOAc, pH 5.0. The purity was finally checked by SDS-PAGE, and only a single band was found.

### Example 8

Construction and Expression of a Site-Directed Variant of Penicillium oxalicum Glucoamylase

Two PCR reactions were performed with plasmid XYZ1471-4, described in Example 7, using primers K79V F and K79VR shown below, which were designed to substitute lysine K at position 79 from the mature sequence to valine (V) and primers F-NP003940 and R-NP003940 shown below, which were designed based on the known sequence and added tags for direct cloning by IN-FUSION™ strategy.

Primer K79V F 18mer
(SEQ ID NO: 25)
GCAGTCTTTCCAATTGAC
Primer K79V R 18mer
(SEQ ID NO: 26)
AATTGGAAAGACTGCCCG
Primer F-NP003940:
(SEQ ID NO: 27)
5′ ACACAACTGGGGATCCACCATGCGTCTCACTCTATTATC
Primer R-NP003940:
(SEQ ID NO: 28)
5′ AGATCTCGAGAAGCTTAAAACTGCCACACGTCGTTGG

The PCR was performed using a PTC-200 DNA Engine under the conditions described below.

| PCR reaction system: | Conditions: | |
|---|---|---|
| 48.5 micro L H2O | 1 | 94° C. 2 min |
| 2 beads puRe Taq Ready-To- | 2 | 94° C. 30 sec |
| Go PCR Beads (Amersham Biosciences) | 3 | 55° C. 30 sec |
| 0.5 micro L X 2100 pmole/micro L Primers | 4 | 72° C. 90 sec |
| (K79V F + Primer R-NP003940, K79V R + | 2-4 | 25 cycles |
| Primer F-NP003940) | 5 | 72° C. 10 min |
| 0.5 micro L Template DNA | | |

DNA fragments were recovered from agarose gel by the Qiagen gel extraction Kit according to the manufacturer's instruction. The resulting purified two fragments were cloned into an Aspergillus expression vector linearized with BamHI and HindIII, using an IN-FUSION™ Dry-Down PCR Cloning Kit (BD Biosciences, Palo Alto, Calif., USA) according to the manufacturer's instructions. The linearized vector construction is as described in WO 2005/042735 A1.

The ligation mixture was used to transform E. coli DH5a cells (TOYOBO). Selected colonies were inoculated in 3 ml of LB medium supplemented with 50 μg of ampicillin per ml and incubated at 37° C. at 225 rpm overnight. Plasmid DNA from the selected colonies was purified using Qiagen plasmid mini kit (Qiagen) according to the manufacturer's instructions. The sequence of Penicillium oxalicum glucoamylase site-directed variant gene sequence was verified before heterologous expression and one of the plasmids was selected for further expression, and was named pPoPE001.

Protoplasts of Aspergillus niger MBin118 were prepared as described in WO 95/02043. One hundred μl of protoplast suspension were mixed with 2.5 μg of the pPoPE001 plasmid and 250 microliters of 60% PEG 4000 (Applichem) (polyethylene glycol, molecular weight 4,000), 10 mM CaCl₂, and 10 mM Tris-HCl pH 7.5 were added and gently mixed. The mixture was incubated at 37° C. for 30 minutes and the protoplasts were mixed with 1% agarose L (Nippon Gene) in COVE sucrose (Cove, 1996, Biochim. Biophys. Acta 133:51-56) supplemented with 10 mM acetamide and 15 mM CsCl and added as a top layer on COVE sucrose plates supplemented with 10 mM acetamide and 15 mM CsCl for transformants selection (4 ml topagar per plate). After incubation for 5 days at 37° C. spores of sixteen transformants were picked up and seed on 750 μl YP-2% Maltose medium in 96 deepwell MT plates. After 5 days of stationary cultivation at 30° C., 10 μl of the culture-broth from each well was analyzed on a SDS-PAGE gel in order to identify the best transformants based on the ability to produce large amount of the glucoamylase.

### Example 9

### Purification of Site-Directed Po AMG Variant PE001

The selected transformant of the variant and the strain expressing the wild type Penicillium oxalicum glucoamylase described in Example 6 was cultivated in 100 ml of YP-2% maltose medium and the culture was filtrated through a 0.22 μm PES filter, and applied on a alpha-cyclodextrin affinity gel column previously equilibrated in 50 mM NaOAc, 150 mM NaCl, pH 4.5 buffer. Unbound materials was washed off the column with equilibration buffer and the glucoamylase was eluted using the same buffer containing 10 mM beta-cyclodextrin over 3 column volumes.

The glucoamylase activity of the eluent was checked to see, if the glucoamylase had bound to the alpha-cyclodextrin affinity gel. The purified glucoamylase samples were then dialyzed against 20 mM NaOAc, pH 5.0.

### Example 10

### Characterization of PE001 Protease Stability

40 μl enzyme solutions (1 mg/ml) in 50 mM sodium acetate buffer, pH 4.5, were mixed with 1/10 volume of 1 mg/ml protease solutions such as aspergillopepsin I described in Biochem J. 1975 April; 147(1):45-53, or the commercially available product from Sigma and aorsin described in Biochemical journal [0264-6021] Ichishima yr: 2003 vol:371 iss:Pt 2 pg:541 and incubated at 4 or 32° C. overnight. As a control experiment, H₂O was added to the sample instead of proteases. The samples were loaded on SDS-PAGE to see if the glucoamylases are cleaved by proteases.

In SDS-PAGE, PE001 only showed one band corresponding to the intact molecule, while the wild type glucoamylase was degraded by proteases and showed a band at lower molecular size at 60 kCa.

TABLE 11
The result of SDS-PAGE after protease treatment

| | Wild type glucoamylase | | | | PE001 | | | | |
|---|---|---|---|---|---|---|---|---|---|
| | Protease | | | | | | | | |
| | aspergillopepsin | | | | aspergillopepsin | | | | |
| | I | | aorsin | | I | | aorsin | | |
| | Incubation temperature (° C.) | | | | | | | | control |
| | 4 | 32 | 4 | 32 | 4 | 32 | 4 | 32 | 4 |
| intact | 100% | 90% | 40% | 10% | 100% | 100% | 100% | 100% | 100% |
| glucoamylase | | | | | | | | | |
| (ca. 70 kDa) | | | | | | | | | |
| cleaved | N.D. | 10% | 60% | 90% | N.D. | N.D. | N.D. | N.D. | N.D. |
| glucoamylase | | | | | | | | | |
| (ca. 60 kDa) | | | | | | | | | |

N.D.: not detected.

### Example 11

### Less Cleavage During Cultivation

Aspergillus transformant of the variant and the wild type Penicillium oxalicum glucoamylase were cultivated in 6-well MT plates containing 4× diluted YP-2% maltose medium supplemented with 10 mM sodium acetate buffer, pH4.5, at 32° C. for 1 week.

The culture supernatants were loaded on SDS-PAGE.

TABLE 12
The result of SDS-PAGE of the culture supernatants

| | Wild type glucoamylase | PE001 |
|---|---|---|
| intact glucoamylase(ca. | 90% | 100% |
| 70 kDa) | | |
| cleaved glucoamylase | 10% | N.D. |
| (ca. 60 kDa) | | |

N.D.: not detected.

The wild type glucoamylase was cleaved by host proteases during fermentation, while the variant yielded only intact molecule.

### Example 12

### Glucoamylase Activity of Variant Compared to Parent

The glucoamylase activity measures as AGU as described above was checked for the purified enzymes of the wild type Penicillium oxalicum and the variant glucoamylase.

The Glucoamylase Unit (AGU) was defined as the amount of enzyme, which hydrolyzes 1 micromole maltose per minute under the standard conditions (37° C., pH 4.3, substrate: maltose 100 mM, buffer: acetate 0.1 M, reaction time 6 minutes).

TABLE 13

| Relative specific activity | AGU/mg |
|---|---|
| Penicillium oxalicum wt | 100% |
| Penicillium oxalicum PE001 (SEQ ID NO: 14 + | 102% |
| K79V substitution) | |

### Example 13

### Purification of Glucoamylase Variants Having Increased Thermostability

The variants showing increased thermostability may be constructed and expressed similar to the procedure described in Example 8. All variants were derived from the PE001. After expression in YPM medium, variants comprising the T65A or Q327F substitution was micro-purified as follows:

Mycelium was removed by filtration through a 0.22 μm filter. 50 μl column material (alpha-cyclodextrin coupled to Mini-Leak divinylsulfone-activated agarose medium according to manufacturer's recommendations) was added to the wells of a filter plate (Whatman, Unifilter 800 μl, 25-30 μm MBPP). The column material was equilibrated with binding buffer (200 mM sodium acetate pH 4.5) by two times addition of 200 μl buffer, vigorous shaking for 10 min (Heidolph, Titramax 101, 1000 rpm) and removal of buffer by vacuum (Whatman, UniVac 3). Subsequently, 400 μl culture supernatant and 100 μl binding buffer was added and the plate incubated 30 min with vigorous shaking. Unbound material was removed by vacuum and the binding step was repeated. Normally 4 wells were used per variant. Three washing steps were then performed with 200 μl buffer of decreasing ionic strength added (50/10/5 mM sodium acetate, pH 4.5), shaking for 15 min and removal of buffer by vacuum. Elution of the bound AMG was achieved by two times addition of 100 μl elution buffer (250 mM sodium acetate, 0.1% alpha-cyclodextrin, pH 6.0), shaking for 15 min and collection of eluted material in a microtiter plate by vacuum. Pooled eluates were concentrated and buffer changed to 50 mM sodium acetate pH 4.5 using centrifugal filter units with 10 kDa cut-off (Millipore Microcon Ultracel YM-10). Micropurified samples were stored at −18° C. until testing of thermostability.

### Example 14

Protein thermal unfolding analysis (TSA, Thermal shift assay).

Protein thermal unfolding of the T65A and Q327F variants, was monitored using Sypro Orange (In-vitrogen, S-6650) and was performed using a real-time PCR instrument (Applied Biosystems; Step-One-Plus).

In a 96-well plate, 25 microliter micropurified sample in 50 mM Acetate pH4,5 at approx. 100 microgram/ml was mixed (5:1) with Sypro Orange (resulting conc.=5×; stock solution from supplier=5000×). The plate was sealed with an optical PCR seal. The PCR instrument was set at a scan-rate of 76° C. pr. hr, starting at 25° C. and finishing at 96° C.

Protein thermal unfolding of the E501V+Y504T variant, was monitored using Sypro Orange (In-vitrogen, S-6650) and was performed using a real-time PCR instrument (Applied Biosystems; Step-One-Plus).

In a 96-well plate, 15 microliter purified sample in 50 mM Acetate pH4,5 at approx. 50 microgram/ml was mixed (1:1) with Sypro Orange (resulting conc.=5×; stock solution from supplier=5000×) with or without 200 ppm Acarbose (Sigma A8980). The plate was sealed with an optical PCR seal. The PCR instrument was set at a scan-rate of 76 degrees C. pr. hr, starting at 25° C. and finishing at 96° C.

Fluorescence was monitored every 20 seconds using in-built LED blue light for excitation and ROX-filter (610 nm, emission).

Tm-values were calculated as the maximum value of the first derivative (dF/dK) (ref.: Gregory et al; J Biomol Screen 2009 14: 700.)

TABLE 14a

| Sample | Tm (Deg. Celsius) +/− 0.4 |
|---|---|
| PO-AMG (PE001) | 80.3 |
| Variant Q327F | 82.3 |
| Variant T65A | 81.9 |

TABLE 14b

| | |
|---|---|
| Sample | Tm (Deg. Celsius) +/−0.4 |

| | | |
|---|---|---|
| Acarbose: | − | + |
| PO-AMG (PE001) | 79.5 | 86.9 |
| Variant E501V Y504T | 79.5 | 95.2 |

### Example 15

### Thermostability Analysis by Differential Scanning Calorimetry (DSC)

Additional site specific variants having substitutions and/or deletions at specific positions were constructed basically as described in Example 8 and purified as described in Example 11.

The thermostability of the purified Po-AMG PE001 derived variants were determined at pH 4.0 or 4.8 (50 mM Sodium Acetate) by Differential Scanning calorimetry (DSC) using a VP-Capillary Differential Scanning calorimeter (MicroCal Inc., Piscataway, N.J., USA). The thermal denaturation temperature, Td (° C.), was taken as the top of the denaturation peak (major endothermic peak) in thermograms (Cp vs. T) obtained after heating enzyme solutions in selected buffers (50 mM Sodium Acetate, pH 4.0 or 4.8) at a constant programmed heating rate of 200K/hr.

Sample- and reference-solutions (approximately 0.3 ml) were loaded into the calorimeter (reference: buffer without enzyme) from storage conditions at 10° C. and thermally pre-equilibrated for 10 minutes at 20° C. prior to DSC scan from 20° C. to 110° C. Denaturation temperatures were determined with an accuracy of approximately +/−1° C.

The isolated variants and the DSC data are disclosed in Table 15 below.

TABLE 15

| | | DSC Td (° C.) @ | DSC Td (° C.) @ |
|---|---|---|---|
| Po-AMG name | Mutations | pH 4.0 | pH 4.8 |
| PE001 (SEQ ID | | 82.1 | 83.4 |
| NO: 14 + K79V) | | | |
| GA167 | E501V Y504T | 82.1 | |
| GA481 | T65A K161S | 84.1 | 86.0 |
| GA487 | T65A Q405T | 83.2 | |
| GA490 | T65A Q327W | 87.3 | |
| GA491 | T65A Q327F | 87.7 | |
| GA492 | T65A Q327Y | 87.3 | |
| GA493 | P11F T65A Q327F | 87.8 | 88.5 |
| GA497 | R1K D3W K5Q G7V N8S T10K P11S | 87.8 | 88.0 |
| | T65A Q327F | | |
| GA498 | P2N P4S P11F T65A Q327F | 88.3 | 88.4 |
| GA003 | P11F D26C K33C T65A Q327F | 83.3 | 84.0 |
| GA009 | P2N P4S P11F T65A Q327W E501V | 88.8 | |
| | Y504T | | |
| GA002 | R1E D3N P4G G6R G7A N8A T10D | 87.5 | 88.2 |
| | P11D T65A Q327F | | |
| GA005 | P11F T65A Q327W | 87.4 | 88.0 |
| GA008 | P2N P4S P11F T65A Q327F E501V | 89.4 | 90.2 |
| | Y504T | | |
| GA010 | P11F T65A Q327W E501V Y504T | | 89.7 |
| GA507 | T65A Q327F E501V Y504T | | 89.3 |
| GA513 | T65A S105P Q327W | | 87.0 |
| GA514 | T65A S105P Q327F | | 87.4 |
| GA515 | T65A Q327W S364P | | 87.8 |
| GA516 | T65A Q327F S364P | | 88.0 |
| GA517 | T65A S103N Q327F | | 88.9 |
| GA022 | P2N P4S P11F K34Y T65A Q327F | | 89.7 |
| GA023 | P2N P4S P11F T65A Q327F D445N | | 89.9 |
| | V447S | | |
| GA032 | P2N P4S P11F T65A I172V Q327F | | 88.7 |
| GA049 | P2N P4S P11F T65A Q327F N502* | | 88.4 |
| GA055 | P2N P4S P11F T65A Q327F N502T | | 88.0 |
| | P563S K571E | | |
| GA057 | P2N P4S P11F R31S K33V T65A | | 89.5 |
| | Q327F N564D K571S | | |
| GA058 | P2N P4S P11F T65A Q327F S377T | | 88.6 |
| GA064 | P2N P4S P11F T65A V325T Q327W | | 88.0 |
| GA068 | P2N P4S P11F T65A Q327F D445N | | 90.2 |
| | V447S E501V Y504T | | |
| GA069 | P2N P4S P11F T65A I172V Q327F | | 90.2 |
| | E501V Y504T | | |
| GA073 | P2N P4S P11F T65A Q327F S377T | | 90.1 |
| | E501V Y504T | | |
| GA074 | P2N P4S P11F D26N K34Y T65A | | 89.1 |
| | Q327F | | |
| GA076 | P2N P4S P11F T65A Q327F I375A | | 90.2 |
| | E501V Y504T | | |
| GA079 | P2N P4S P11F T65A K218A K221D | | 90.9 |
| | Q327F E501V Y504T | | |
| GA085 | P2N P4S P11F T65A S103N Q327F | | 91.3 |
| | E501V Y504T | | |
| GA086 | P2N P4S T10D T65A Q327F E501V | | 90.4 |
| | Y504T | | |
| GA088 | P2N P4S F12Y T65A Q327F E501V | | 90.4 |
| | Y504T | | |
| GA097 | K5A P11F T65A Q327F E501V | | 90.0 |
| | Y504T | | |
| GA101 | P2N P4S T10E E18N T65A Q327F | | 89.9 |
| | E501V Y504T | | |
| GA102 | P2N T10E E18N T65A Q327F E501V | | 89.8 |
| | Y504T | | |
| GA084 | P2N P4S P11F T65A Q327F E501V | | 90.5 |
| | Y504T T568N | | |
| GA108 | P2N P4S P11F T65A Q327F E501V | | 88.6 |
| | Y504T K524T G526A | | |
| GA126 | P2N P4S P11F K34Y T65A Q327F | | 91.8 |
| | D445N V447S E501V Y504T | | |
| GA129 | P2N P4S P11F R31S K33V T65A | | 91.7 |
| | Q327F D445N V447S E501V Y504T | | |
| GA087 | P2N P4S P11F D26N K34Y T65A | | 89.8 |
| | Q327F E501V Y504T | | |
| GA091 | P2N P4S P11F T65A F80* Q327F | | 89.9 |
| | E501V Y504T | | |
| GA100 | P2N P4S P11F T65A K112S Q327F | | 89.8 |
| | E501V Y504T | | |
| GA107 | P2N P4S P11F T65A Q327F E501V | | 90.3 |
| | Y504T T516P K524T G526A | | |
| GA110 | P2N P4S P11F T65A Q327F E501V | | 90.6 |
| | N502T Y504* | | |

### Example 16

Thermostability Analysis by Thermo-Stress Test and pNPG Assay

Starting from one of the identified substitution variants from Example 15, identified as GA008, additional variants were tested by a thermo-stress assay in which the supernatant from growth cultures were assayed for glucoamylase (AMG) activity after a heat shock at 83° C. for 5 min.

After the heat-shock the residual activity of the variant was measured as well as in a non-stressed sample.

Description of Po-AMG pNPG Activity Assay:

The Penicillium oxalicum glucoamylase pNPG activity assay is a spectrometric endpoint assay where the samples are split in two and measured thermo-stressed and non-thermo-stressed. The data output is therefore a measurement of residual activity in the stressed samples.

### Growth:

A sterile micro titer plate (MTP) was added 200 μL rich growth media (FT X-14 without Dowfax) to each well. The strains of interest were inoculated in triplicates directly from frozen stocks to the MTP. Benchmark was inoculated in 20 wells. Non-inoculated wells with media were used as assay blanks. The MTP was placed in a plastic box containing wet tissue to prevent evaporation from the wells during incubation. The plastic box was placed at 34° C. for 4 days.

### Assay:

50 μL supernatant was transferred to 50 μL 0.5 M NaAc pH 4.8 to obtain correct sample pH.

50 μL dilution was transferred to a PCR plate and thermo-stressed at 83° C. for 5 minutes in a PCR machine. The remaining half of the dilution was kept at RT.

20 μL of both stressed and unstressed samples was transferred to a standard MTP. 20 μL pNPG-substrate was added to start the reaction. The plate was incubated at RT for 1 hour.

The reaction was stopped and the colour developed by adding 50 μL 0.5M Na₂CO₃. The yellow colour was measured on a plate reader (Molecular Devices) at 405 nm.

### Buffers:

### 0.5 M NaAc pH 4.8

### 0.25 M NaAc pH 4.8

Substrate, 6 mM pNPG:
15 mg 4-nitrophenyl D-glucopyranoside in 10 mL 0.25 NaAc pH 4.8
Stop/developing solution:
0.5 M Na₂CO₃
Data treatment:

In Excel the raw Abs405 data from both stressed and unstressed samples were blank subtracted with their respective blanks. The residual activity (% res. act.=(Abs_{unstressed}−(Abs_{unstressed}−Abs_{stressed}))/Abs_{unstressed}*100%) was calculated and plotted relative to benchmark, Po-amg0008.

TABLE 16

| Po-AMG name | Mutations | % residual activity |
|---|---|---|
| GA008 | P2N P4S P11F T65A Q327F | 100 |
| | E501V Y504T | |
| GA085 | P2N P4S P11F T65A S103N | 127 |
| | Q327F E501V Y504T | |
| GA097 | K5A P11F T65A Q327F | 106 |
| | E501V Y504T | |
| GA107 | P2N P4S P11F T65A Q327F | 109 |
| | E501V Y504T T516P K524T | |
| | G526A | |
| GA130 | P2N P4S P11F T65A V79A | 111 |
| | Q327F E501V Y504T | |
| GA131 | P2N P4S P11F T65A V79G | 112 |
| | Q327F E501V Y504T | |
| GA132 | P2N P4S P11F T65A V79I | 101 |
| | Q327F E501V Y504T | |
| GA133 | P2N P4S P11F T65A V79L | 102 |
| | Q327F E501V Y504T | |
| GA134 | P2N P4S P11F T65A V79S | 104 |
| | Q327F E501V Y504T | |
| GA150 | P2N P4S P11F T65A L72V | 101 |
| | Q327F E501V Y504T | |
| GA155 | S255N Q327F E501V Y504T | 105 |

TABLE 17

| Po-AMG name | Mutations | % residual activity |
|---|---|---|
| GA008 | P2N P4S P11F T65A Q327F | 100 |
| | E501V Y504T | |
| GA179 | P2N P4S P11F T65A E74N | 108 |
| | V79K Q327F E501V Y504T | |
| GA180 | P2N P4S P11F T65A G220N | 108 |
| | Q327F E501V Y504T | |
| GA181 | P2N P4S P11F T65A Y245N | 102 |
| | Q327F E501V Y504T | |
| GA184 | P2N P4S P11F T65A Q253N | 110 |
| | Q327F E501V Y504T | |
| GA185 | P2N P4S P11F T65A D279N | 108 |
| | Q327F E501V Y504T | |
| GA186 | P2N P4S P11F T65A Q327F | 108 |
| | S359N E501V Y504T | |
| GA187 | P2N P4S P11F T65A Q327F | 102 |
| | D370N E501V Y504T | |
| GA192 | P2N P4S P11F T65A Q327F | 102 |
| | V460S E501V Y504T | |
| GA193 | P2N P4S P11F T65A Q327F | 102 |
| | V460T P468T E501V Y504T | |
| GA195 | P2N P4S P11F T65A Q327F | 103 |
| | T463N E501V Y504T | |
| GA196 | P2N P4S P11F T65A Q327F | 106 |
| | S465N E501V Y504T | |
| GA198 | P2N P4S P11F T65A Q327F | 106 |
| | T477N E501V Y504T | |

### Example 17

### Test for Glucoamylase Activity of Thermo-Stable Variants

All of the above described variants disclosed in tables 15, 16, and 17 have been verified for Glucoamylase activity on culture supernatants using the pNPG assay described in Example 16.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

### Example 18

### Addition of Acetate to Ethanol Fermentations

The purpose of this experiment was to evaluate the fermentation performance of Ethanol Red™ in the presence of varying levels of acetate.

### Corn Mash

Industrially prepared corn mash was obtained from CornLP (Liquozyme™ SODS liquefaction). Solids on this mash were measured to be 34.17% by 105° C. drying oven.

### Yeast Strains and Preparation

The yeast strain tested in this experiment was Ethanol Red™ (Fermentis). Yeast was rehydrated by weighing 2.08 g of dried yeast into 40 ml of 36.5° C. tap water in a 125 mL Erlenmeyer flask. The flasks were then covered with parafilm and allowed to incubate in a 36.5° C. water bath. After 15 minutes, the flasks were swirled, but no other agitation took place. After a total of 30 minutes, the flasks were removed from the water bath. Total yeast concentration was determined using the YC-100 in duplicate.

### Simultaneous Saccharification and Fermentation (SSF)

Lactrol™ (PhibroChem) was added to each mash to a final concentration of 24 ppm. The pH after liquefaction and acetic acid addition was adjusted to 5.3 for SSF. Urea was adjusted to 600 ppm and water added to maintain a consistent solids level between mashes. Approximately 5 grams of each of the resulting mashes was transferred to test tubes having a 1/64 hole drilled in the top to allow CO₂ release. Glucoamylase SA (“GSA”)/Cellulase VD (“CVD”) enzyme blend was dosed to each tube of mash at 110 μg EP GSA/gDS and 30 μg EP CVD/gDS. Yeast was dosed at 5×10e6 cells/g mash. Milli-Q water was added to each tube so that a total volume of liquid added (enzyme+MQ water+acid) to each tube would be equally proportionate to the mash weight. Fermentations took place in a 32° C. water bath for 54 hours. Samples were vortexed periodically (in the morning and in the evening) throughout the fermentation. Acetate in a range between 0 and 120 ppm was added prior to inoculation (i.e., before exponential growth).

### HPLC Analysis

Fermentation sampling took place after 48 and 54 hours of fermentation by sacrificing 3 tubes per treatment. Each tube was processed for HPLC analysis by deactivation with 150 μL of 40% v/v H₂SO₄, vortexing, centrifuging at 1460×g for 10 minutes, and filtering through a 0.45 μm Whatman PP filter. All samples were processed without further dilution. Samples were stored at 4° C. prior to and during HPLC analysis.

TABLE 18
HPLC System

| | |
|---|---|
| HPLC | Agilent's 1100/1200 series with Chem station software |
| System | Degasser, Quaternary Pump, Auto-Sampler, Column |
| | Compartment /w Heater |
| | Refractive Index Detector (RI) |
| Column | Bio-Rad HPX-87H Ion Exclusion Column 300 mm × 7.8 mm |
| | part#125-0140 |
| | Bio-Rad guard cartridge Cation H part# 125-0129, |
| | Holder part# 125-0131 |
| Method | 0.005M H2SO4 mobile phase |
| | Flow rate: 0.6 ml/min |
| | Column temperature: 65° C. |
| | RI detector temperature: 55° C. |

Samples were analyzed for sugars (DP4+, DP3, DP2, glucose, and fructose), organic acids (lactic and acetic), glycerol, and ethanol.

### Results

Ethanol titers over a range of concentrations of added acetate can be seen in FIG. 1 and Table 19 below. It can be seen from FIG. 1 that adding between 5 mM and 60 mM acetate increases ethanol titers between 0.27 and 2.71%.

TABLE 19
Ethanol Titers and Comparisons to Fermentations with no Added Acetate

| | | % Boost | | % Boost |
|---|---|---|---|---|
| Added | 48 Hour | over no | 54 Hour | over no |
| Acetate | ETOH | added | ETOH | added |
| (mM) | (w/v %) | acetate | (w/v %) | acetate |
| 0 | 13.51 | 0.00 | 13.58 | 0.00 |
| 5 | 13.55 | 0.28 | 13.71 | 0.97 |
| 10 | 13.66 | 1.07 | 13.73 | 1.10 |
| 20 | 13.77 | 1.88 | 13.83 | 1.90 |
| 40 | 13.82 | 2.29 | 13.83 | 1.84 |
| 60 | 13.88 | 2.71 | 13.83 | 1.84 |
| 120 | 12.92 | −4.41 | 12.94 | −4.68 |

Glycerol levels were reduced with added acetate. FIG. 2 and Table 20 below show glycerol titers over a range of added acetate concentrations.

TABLE 20
Glycerol Titers and comparisons to fermentations with no added acetate

| | | % | | % |
|---|---|---|---|---|
| | | change | | change |
| Added | 48 Hour | over no | 54 Hour | over no |
| Acetate | Glycerol | added | Glycerol | added |
| (mM) | (w/v %) | acetate | (w/v %) | acetate |
| 0 | 1.716541 | 0.00 | 1.689756 | 0.00 |
| 5 | 1.636248 | −4.68 | 1.624543 | −3.86 |
| 10 | 1.552997 | −9.53 | 1.568505 | −7.18 |
| 20 | 1.461697 | −14.85 | 1.449637 | −14.21 |
| 40 | 1.364985 | −20.48 | 1.365133 | −19.21 |
| 60 | 1.332045 | −22.40 | 1.323093 | −21.70 |
| 120 | 1.337295 | −22.09 | 1.340395 | −20.68 |

### Example 19

### Addition of Benzoate, Propionate, and Formate to Ethanol Fermentations Using Ethanol Red™

The purpose of this experiment was to evaluate the fermentation performance of Ethanol Red™ in the presence of varying levels of multiple weak acids (Benzoate pKa: 4.20; Propionate pKa: 4.88; and Formate pKa: 3.77).

### Corn Mash

Industrially prepared corn mash was obtained from Lincolnland (Liquozyme™ SODS liquefaction). Solids on this mash were measured to be 31.3% by moisture balance.

### Yeast Strains and Preparation

The yeast strain tested in this experiment was Ethanol Red™ (Fermentis). Yeast was rehydrated by weighing 2.08 g of dried yeast into 40 ml of 36.5° C. tap water in a 125 mL Erlenmeyer flask. The flasks were then covered with parafilm and allowed to incubate in a 36.5° C. water bath. After 15 minutes, the flasks were swirled, but no other agitation took place. After a total of 30 minutes, the flasks were removed from the water bath. Total yeast concentration was determined using the YC-100 in duplicate.

### Simultaneous Saccharification and Fermentation (SSF)

Lactrol (PhibroChem) was added to each mash to a final concentration of 24 ppm. The pH after liquefaction was 4.9 and was adjusted to various pHs for SSF. Urea was adjusted to 600 ppm and water added to maintain a consistent solids level between mashes. Approximately 5 grams of each of the resulting mashes was transferred to test tubes having a 1/64 hole drilled in the top to allow CO₂ release. Glucoamylase SA (“GSA”)/Cellulase VD (“CVD”) enzyme blend was dosed to each tube of mash at 110 μg EP GSA/gDS and 30 μg EP CVD/gDS. Yeast was dosed at 5×10e6 cells/g mash. Milli-Q water was added to each tube so that a total volume of liquid added (enzyme+MQ water) to each tube would be equally proportionate to the mash weight. Fermentations took place in a 32° C. water bath for 54 hours. Samples were vortexed periodically (in the morning and in the evening) throughout the fermentation. Benzoic acid was added in the range of 0-0.8 mM. Propionic and Formic acid were added in the range of 0-30 mM. All acid additions were done prior to inoculation (i.e., before exponential growth).

### HPLC Analysis

Fermentation sampling took place after 54 hours of fermentation by sacrificing 3 tubes per treatment. Each tube was processed for HPLC analysis by deactivation with 150 μL of 40% v/v H₂SO₄, vortexing, centrifuging at 1460×g for 10 minutes, and filtering through a 0.45 μm Whatman PP filter. All samples were processed without further dilution. Samples were stored at 4° C. prior to and during HPLC analysis.

TABLE 21
HPLC System

| | |
|---|---|
| HPLC | Agilent's 1100/1200 series with Chem station software |
| System | Degasser, Quaternary Pump, Auto-Sampler, Column |
| | Compartment /w Heater |
| | Refractive Index Detector (RI) |
| Column | Bio-Rad HPX-87H Ion Exclusion Column 300 mm × 7.8 mm |
| | part# 125-0140 |
| | Bio-Rad guard cartridge Cation H part# 125-0129, |
| | Holder part# 125-0131 |
| Method | 0.005M H2SO4 mobile phase |
| | Flow rate: 0.6 ml/min |
| | Column temperature: 65° C. |
| | RI detector temperature: 55° C. |

Samples were analyzed for sugars (DP4+, DP3, DP2, glucose, and fructose), organic acids (lactic and acetic), glycerol, and ethanol.

### Results

FIG. 3 and Table 22 below show the results of adding low levels of benzoic acid to ethanol fermentations. The addition of small amounts of benzoic acid increases fermentation performance.

TABLE 22
Ethanol Titers in response to Benzoic Acid and comparisons to
fermentations with no added acid

| | | % | | |
|---|---|---|---|---|
| | | Boost | | |
| | | over | | % Boost |
| | ETOH | no | ETOH | over no |
| | pH 3.8 | added | pH 5 | added |
| Concentration | (w/v %) | acid | (w/v %) | acid |
| 0 | 13.36713 | 0.00 | 12.97801 | 0.00 |
| 0.2 | 13.72779 | 2.70 | 13.01312 | 0.27 |
| 0.5 | 13.6785 | 2.33 | 13.23297 | 1.96 |
| 0.8 | 13.35147 | −0.12 | 13.39321 | 3.20 |

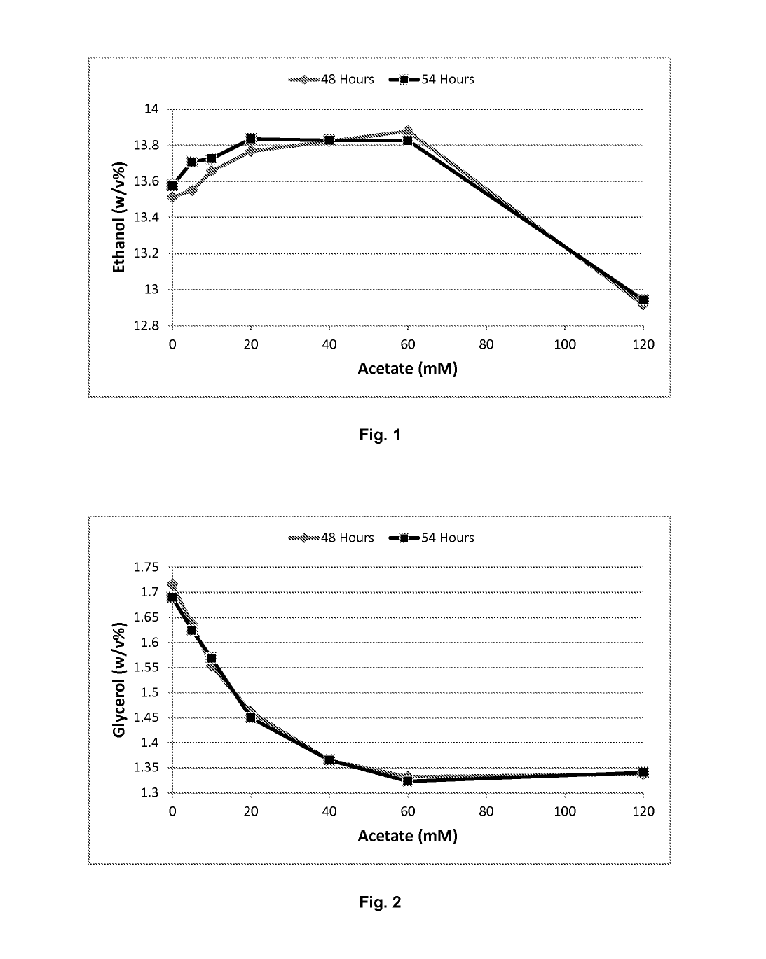

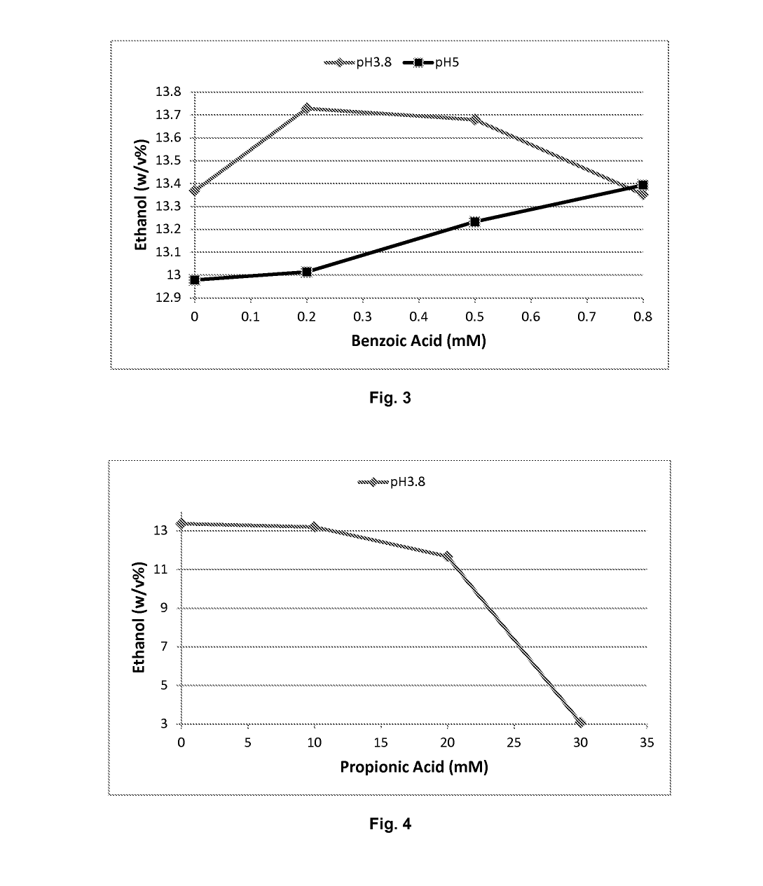

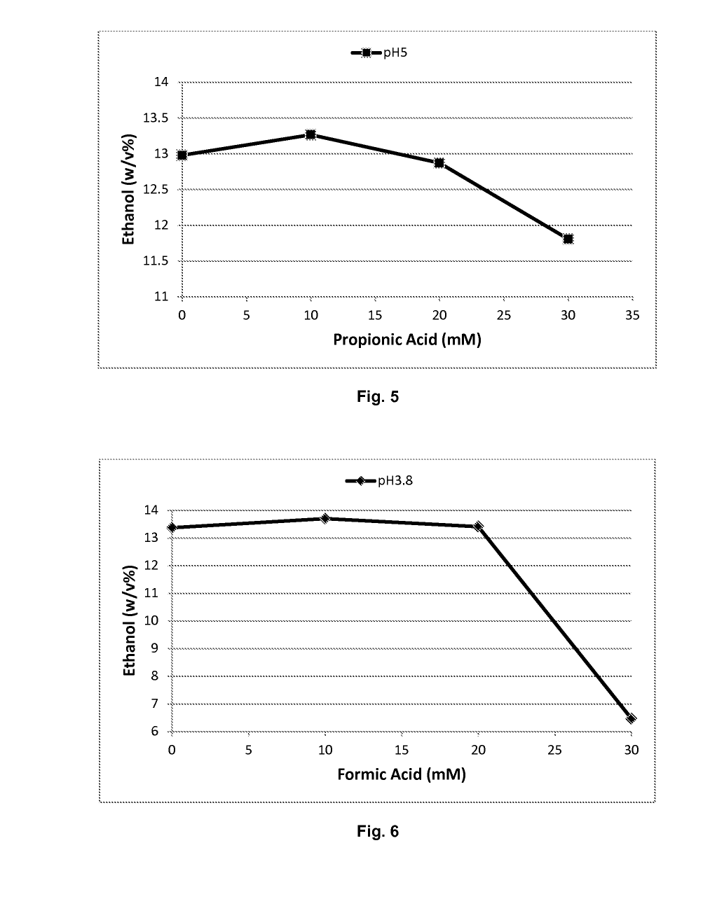

FIGS. 4 and 5 and Table 23 below show the results of adding propionic acid to fermentations. This effect appears to be pH sensitive as at pH3.8 there is a negative effect, but at pH5, 10 mM addition boosts ethanol production 2.2%.

TABLE 23
Ethanol Titers after propionic acid addition and comparison
to fermentations with no added acid

| | | % | | % |
|---|---|---|---|---|
| | | Boost | | Boost |
| | ETOH | over no | ETOH | over no |
| | pH 3.8 | added | pH 5 | added |
| Concentration | (w/v %) | acid | (w/v %) | acid |
| 0 | 13.36713 | 0 | 12.97801 | 0.00 |
| 10 | 13.19729 | −1.27 | 13.26472 | 2.21 |
| 20 | 11.66807 | −12.7 | 12.86955 | −0.84 |
| 30 | 3.063552 | −77.08 | 11.8064 | −9.03 |

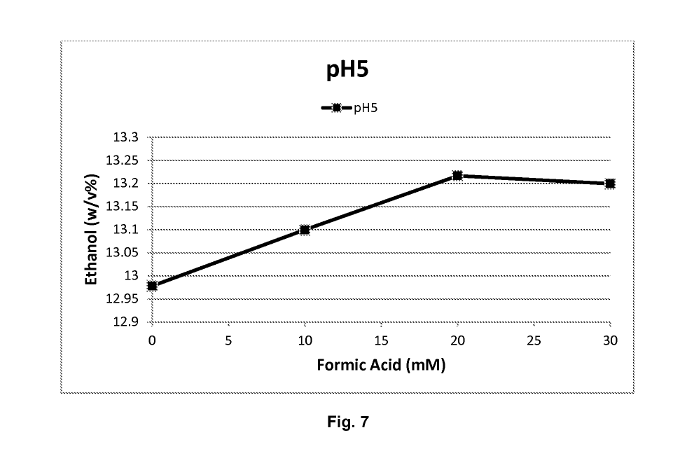

FIGS. 6 and 7 and Table 24 below show the effect of adding formic acid to fermentations. There is a boost seen with addition of formic acid, the level of which is highly dependent on fermentation starting pH.

TABLE 24
Ethanol Titers after Formic acid addition and comparison
to fermentations with no added acid

| | | % Boost | | % Boost |
|---|---|---|---|---|
| | ETOH | over no | ETOH | over no |
| | pH 3.8 | added | pH 5 | added |
| Concentration | (w/v %) | acid | (w/v %) | acid |
| 0 | 13.36713 | 0.00 | 12.97801 | 0.00 |
| 10 | 13.69456 | 2.45 | 13.09951 | 0.94 |
| 20 | 13.40804 | 0.31 | 13.21645 | 1.84 |
| 30 | 6.458078 | −51.69 | 13.19922 | 1.70 |

Glycerol levels dropped with the additional of all three tested acids.

TABLE 25
Glycerol Titers after weak acid addition and comparison to
fermentations with no added acid.

| | | | % | | % |
|---|---|---|---|---|---|
| | | | Change | | Change |
| | | Glycerol | from no | Glycerol | from no |
| | Concentration | pH 3.8 | added | pH 5 | added |
| | (mM) | (w/v %) | acid | (w/v %) | acid |
| Benzoic | 0 | 1.309 | 0 | 1.343 | 0 |
| Acid | 0.2 | 1.159 | −11.47 | 1.160 | −13.60 |
| | 0.5 | 1.091 | −16.65 | 1.127 | −16.08 |
| | 0.8 | 1.041 | −20.45 | 1.093 | −18.58 |
| Propionic | 0 | 1.3090 | 0 | 1.343 | 0 |
| Acid | 10 | 0.985 | −24.78 | 1.017 | −24.26 |
| | 20 | 0.896 | −31.55 | 0.927 | −30.95 |
| | 30 | 0.654 | −50.02 | 0.874 | −34.94 |
| Formic | 0 | 1.309 | 0 | 1.343 | 0 |
| Acid | 10 | 1.078 | −17.68 | 1.175 | −12.49 |
| | 20 | 1.070 | −18.24 | 1.117 | −16.81 |
| | 30 | 0.966 | −26.19 | 1.111 | −17.26 |

### Example 20

### Acetic Acid Addition in Tube Scale RSH Ethanol Fermentations

### Mash Preparation

Yellow dent corn (obtained from Lincolnway on 19 Sep. 2013 and ground in-house on a Bunn coffee grinder to a mean particle size around 250 microns) was mixed with tap water and the dry solids (DS) level was determined to be 34.30% by moisture balance. This mixture was supplemented with 3 ppm penicillin and 500 ppm urea. The slurry was adjusted to pH 4.5 with 40% H₂SO₄.

### Yeast Strains and Preparation

The yeast strain tested in this experiment was Ethanol Red™ (Fermentis). Yeast was rehydrated by weighing 2.75 g of dried yeast into 50 ml of 36.5° C. tap water in a 125 mL Erlenmeyer flask. The flasks were then covered with parafilm and allowed to incubate in a 36.5° C. water bath. After 15 minutes, the flasks were swirled, but no other agitation took place. After a total of 30 minutes, the flasks were removed from the water bath.

### Simultaneous Saccharification and Fermentation (SSF)

Approximately 5 grams of mash was transferred to test tubes having a 1/64 hole drilled in the top to allow CO₂ release. PsAMG/AAPE096 (ratio of PsAMG to AAPE096 was 33.5) was dosed to each tube of mash at 0.85 AGU/gDS or RSH Blend P was dosed at 0.32 AGU/gDS. Yeast was dosed at 10e6 cells/g mash. Milli-Q water was added to each tube so that a total volume of liquid added (enzyme+MQ water) to each tube would be equally proportionate to the mash weight. Fermentations took place in a 32° C. water bath for 88 hours. Samples were vortexed periodically (in the morning and in the evening) throughout the fermentation.

### HPLC Analysis

Fermentation sampling took place after 72 and 88 hours of fermentation by sacrificing 3 tubes per treatment. Each tube was processed for HPLC analysis by deactivation with 50 μL of 40% v/v H₂SO₄, vortexing, centrifuging at 1460×g for 10 minutes, and filtering through a 0.45 μm Whatman PP filter. All samples were processed without further dilution. Samples were stored at 4° C. prior to and during HPLC analysis.

TABLE 26
HPLC System

| | |
|---|---|
| HPLC | Agilent's 1100/1200 series with Chem station software |
| System | Degasser, Quaternary Pump, Auto-Sampler, Column |
| | Compartment /w Heater |
| | Refractive Index Detector (RI) |
| Column | Bio-Rad HPX-87H Ion Exclusion Column 300 mm × 7.8 mm |
| | part# 125-0140 |
| | Bio-Rad guard cartridge Cation H part# 125-0129, |
| | Holder part# 125-0131 |
| Method | 0.005M H2SO4 mobile phase |
| | Flow rate: 0.6 ml/min |
| | Column temperature: 65° C. |
| | RI detector temperature: 55° C. |

Samples were analyzed for sugars (DP4+, DP3, DP2, glucose, and fructose), organic acids (lactic and acetic), glycerol, and ethanol.

### Results

The addition of acetic acid to a concentration of 25 mM boosted the performance of Ethanol Red™ between 2.8 and 4.2% depending on time and enzyme used. This data can be found in Table 27 below.

TABLE 27
Effect of Acetic Acid addition on RSH fermentation performance.

| | 72 Hour Data | | | 88 Hour Data | | |
|---|---|---|---|---|---|---|
| | | 25 mM | % | | 25 mM | % |
| | Control | Acetate | Boost | Control | Acetate | Boost |
| PsAMG/AAPE096 | 161.77 | 166.41 | 2.87 | 162.76 | 167.46 | 2.88 |
| RSH Blend P | 156.68 | 163.29 | 4.22 | 160.89 | 165.78 | 3.04 |

### The Invention is Described in the Following Numbered Paragraphs.

1. A process for producing a fermentation product from starch-containing material comprising the steps of:
i) liquefying the starch-containing material at a temperature above the initial gelatinization temperature using an alpha-amylase;
ii) saccharifying using a glucoamylase;
iii) fermenting using a fermenting organism;
wherein an acid having a pKa in the range from 3.75 to 5.75 is present and/or added in fermentation so that the acid concentration in fermentation is maintained between above 0 (zero) and 100 mmoles/L fermentation medium and wherein the acid is added before the exponential growth phase of the fermenting organism.
2. The process of paragraph 1, wherein the fermenting organism is yeast, preferably derived from a strain of Saccharomyces, such as a strain of Saccharomyces cerevisiae.
3. The process of paragraph 1 or 2, wherein the fermenting organism is a strain of baker's yeast, such as ETHANOL RED™ (“ER”).
4. The process of any of paragraph 1-3, wherein the acid concentration in fermentation is maintained 5 and 80 mmoles/L, or preferably between 10 and 100 mmoles/L fermentation medium
5. The process of any of paragraphs 1-4, wherein the acid is added during lag phase.
6. The process of any of paragraphs 1-5, wherein the acid has a pKa in the range from 4.0 to 5.0.
7. The process of any of paragraphs 1-5, wherein the acid is selected from the group of acetic acid, benzoic acid, propionic acid, formic acid, sorbic acid and succinic acid.
8. The process of paragraphs 1-6, wherein the acid concentration is in fermemntation is between 20-80 mmoles/L in case the acid is acetic acid.
9. The process of any of paragraphs 1-8, wherein the acid hydrophobic when protonated.
10. The process of any of paragraphs 1-9, wherein the fermentation product is an alcohol, preferably ethanol, especially fuel ethanol, potable ethanol and/or industrial ethanol.
11. The process of any of paragraphs 1-10, wherein a nitrogen source, preferably urea, is added in saccharification, fermentation, or simultaneous saccharification and fermentation (SSF).
12. The process of any of paragraphs 1-12, further comprises, prior to the liquefaction step i), the steps of:

x) reducing the particle size of the starch-containing material, preferably by dry milling;

y) forming a slurry comprising the starch-containing material and water.

13. The process of any of paragraphs 1-12, wherein at least 50%, preferably at least 70%, more preferably at least 80%, especially at least 90% of the starch-containing material fit through a sieve with #6 screen.
14. The process of any of paragraphs 1-13, wherein the pH in liquefaction is between 4-7, such as between pH 4.5-6,5, such as between pH 5.0-6.5, such as between pH 5.0-6.0, such as between pH 5.2-6.2, such as around 5.2, such as around 5.4, such as around 5.6, such as around 5.8.
15. The process of any of paragraphs 1-14, wherein the temperature in liquefaction is in the range from 70-100° C., such as between 75-95° C., such as between 75-90° C., preferably between 80-90° C., such as 82-88° C., such as around 85° C.
16. The process of any of paragraphs 1-15, wherein a jet-cooking step is carried out prior to liquefaction in step i).
17. The process of paragraph 16, wherein the jet-cooking is carried out at a temperature between 110-145° C., preferably 120-140° C., such as 125-135° C., preferably around 130° C. for about 1-15 minutes, preferably for about 3-10 minutes, especially around about 5 minutes.
18. The process of any of paragraphs 1-17, wherein saccharification and fermentation is carried out sequentially or simultaneously (SSF).
19. The process of any of paragraphs 1-18, wherein saccharification is carried out at a temperature from 20-75° C., preferably from 40-70° C., such as around 60° C., and at a pH between 4 and 5.
20. The process of any of paragraphs 1-19, wherein fermentation or simultaneous saccharification and fermentation (SSF) is carried out carried out at a temperature from 25° C. to 40° C., such as from 28° C. to 35° C., such as from 30° C. to 34° C., preferably around about 32° C. In an embodiment fermentation is ongoing for 6 to 120 hours, in particular 24 to 96 hours.
21. The process of any of paragraphs 1-21, wherein the fermentation product is recovered after fermentation, such as by distillation.
22. The process of any of paragraphs 1-21, wherein the starch-containing starting material is whole grains.
23. The process of any of paragraphs 1-22, wherein the starch-containing material is derived from corn, wheat, barley, rye, milo, sago, cassava, manioc, tapioca, sorghum, rice or potatoes.
24. The process of any of paragraphs 1-23, wherein the alpha-amylase used or added in liquefaction step i) is of bacterial origin.
25. The process of any of paragraphs 1-24, wherein the alpha-amylase is from the genus Bacillus, such as a strain of Bacillus stearothermophilus, in particular a variant of a Bacillus stearothermophilus alpha-amylase, such as the one shown in SEQ ID NO: 3 in WO 99/019467 or SEQ ID NO: 1 herein.
26. The process of paragraph 25, wherein the Bacillus stearothermophilus alpha-amylase or variant thereof is truncated, preferably to have from 485-495 amini acuds, such as around 491 amino acids.
27. The process of any of paragraphs 25 or 26, wherein the Bacillus stearothermophilus alpha-amylase has a double deletion at positions I181+G182, and optionally a N193F substitution, or deletion of R179+G180 (using SEQ ID NO: 1 for numbering).
28. The process of any of paragraphs 25-27, wherein the Bacillus stearothermophilus alpha-amylase has a substitution in position S242, preferably S242Q substitution (using SEQ ID NO: 1 for numbering).
29. The process of any of paragraphs 25-28, wherein the Bacillus stearothermophilus alpha-amylase has a substitution in position E188, preferably E188P substitution (using SEQ ID NO: 1 for numbering).
30. The process of any of paragraphs 1-29, wherein the alpha-amylase has a T½ (min) at pH 4.5, 85° C., 0.12 mM CaCl₂) of at least 10, such as at least 15, such as at least 20, such as at least 25, such as at least 30, such as at least 40, such as at least 50, such as at least 60, such as between 10-70, such as between 15-70, such as between 20-70, such as between 25-70, such as between 30-70, such as between 40-70, such as between 50-70, such as between 60-70.
31. The process of any of paragraphs 1-30, wherein the alpha-amylase present and/or added in liquefaction step i) is selected from the group of Bacillus stearothermophilus alpha-amylase variants with the following mutations in addition to I181*+G182*, and optionally N193F:
V59A+Q89R+G112D+E129V+K177L+R179E+K220P+N224L+Q254S;
V59A+Q89R+E129V+K177L+R179E+H208Y+K220P+N224L+Q254S;
V59A+Q89R+E129V+K177L+R179E+K220P+N224L+Q254S+D269E+D281N;
V59A+Q89R+E129V+K177L+R179E+K220P+N224L+Q254S+1270L;
V59A+Q89R+E129V+K177L+R179E+K220P+N224L+Q254S+H274K;
V59A+Q89R+E129V+K177L+R179E+K220P+N224L+Q254S+Y276F;
V59A+E129V+R157Y+K177L+R179E+K220P+N224L+S242Q+Q254S;
V59A+E129V+K177L+R179E+H208Y+K220P+N224L+S242Q+Q254S;
59A+E129V+K177L+R179E+K220P+N224L+S242Q+Q254S;
V59A+E129V+K177L+R179E+K220P+N224L+S242Q+Q254S+H274K;
V59A+E129V+K177L+R179E+K220P+N224L+S242Q+Q254S+Y276F;
V59A+E129V+K177L+R179E+K220P+N224L+S242Q+Q254S+D281N;
V59A+E129V+K177L+R179E+K220P+N224L+S242Q+Q254S+M284T;
V59A+E129V+K177L+R179E+K220P+N224L+S242Q+Q254S+G416V;
V59A+E129V+K177L+R179E+K220P+N224L+Q254S;
V59A+E129V+K177L+R179E+K220P+N224L+Q254S+M284T;
A91 L+M961+E129V+K177L+R179E+K220P+N224L+S242Q+Q254S;
E129V+K177L+R179E;
E129V+K177L+R179E+K220P+N224L+S242Q+Q254S;
E129V+K177L+R179E+K220P+N224L+S242Q+Q254S+Y276F+L427M;
E129V+K177L+R179E+K220P+N224L+S242Q+Q254S+M284T;
E129V+K177L+R179E+K220P+N224L+S242Q+Q254S+N376*+1377*;
E129V+K177L+R179E+K220P+N224L+Q254S;
E129V+K177L+R179E+K220P+N224L+Q254S+M284T;
E129V+K177L+R179E+S242Q;
E129V+K177L+R179V+K220P+N224L+S242Q+Q254S;
K220P+N224L+S242Q+Q254S;
M284V;
V59A+Q89R+E129V+K177L+R179E+Q254S+M284V.
V59A+E129V+K177L+R179E+Q254S+M284V;
   32. The process of any of paragraphs 1-31, wherein the alpha-amylase present and/or added in liquefaction step i) is selected from the following group of Bacillus stearothermophilus alpha-amylase variants:
I181*+G182*+N193F+E129V+K177L+R179E;
I181*+FG182*+N193F+V59A+Q89R+E129V+K177L+R179E+H208Y+K220P+N224L+Q254S
I181*+G182*+N193F+V59A+Q89R+E129V+K177L+R179E+Q254S+M284V;
I181*+G182*+N193F+V59A+E129V+K177L+R179E+Q254S+M284V and
I181*+G182*+N193F+E129V+K177L+R179E+K220P+N224L+S242Q+Q254S (using SEQ ID NO: 1 herein for numbering).
   33. The process of any of paragraphs 1-32, wherein a glucoamylase is present and/or added in saccharification and/or fermentation.
   34. The process of paragraph 33, wherein the glucoamylase present and/or added in saccharification, fermentation or simultaneous saccharification and fermentation (SSF) is of fungal origin, preferably from a strain of Aspergillus, preferably A. niger, A. awamori, or A. oryzae; or a strain of Trichoderma, preferably T. reesei; or a strain of Talaromyces, preferably T. emersonii, or a strain of Pycnoporus, or a strain of Gloephyllum, such as G. serpiarium or G. trabeum, or a strain of the Nigrofomes.
   35. The process of any of paragraphs 1-34, wherein the glucoamylase is derived from Talaromyces emersonii, such as the one shown in SEQ ID NO: 19 herein,
   36. The process of any of paragraphs 1-35, wherein the glucoamylase is selected from the group consisting of:
   (i) a glucoamylase comprising the mature polypeptide of SEQ ID NO: 19 herein;
   (ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the mature polypeptide of SEQ ID NO: 19 herein.
   37. The process of any of paragraphs 1-36, wherein the glucoamylase present and/or added in saccharification is derived from Gloephyllum serpiarium, such as the one shown in SEQ ID NO: 15 herein.
   38. The process of any of paragraphs 1-7, wherein the glucoamylase present and/or added in saccharification is selected from the group consisting of:
   (i) a glucoamylase comprising the mature polypeptide of SEQ ID NO: 15 herein;
   (ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the mature polypeptide of SEQ ID NO: 15 herein.
   39. The process of any of paragraphs 1-38, wherein the glucoamylase present and/or added in saccharification is derived from Gloeophyllum trabeum such as the one shown in SEQ ID NO: 17 herein.
   40. The process of any of paragraphs 1-39, wherein the glucoamylase present and/or added in saccharification is selected from the group consisting of:
   (i) a glucoamylase comprising the mature polypeptide of SEQ ID NO: 17 herein;
   (ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the mature polypeptide of SEQ ID NO: 17 herein.
   41. The process of any of paragraphs 1-39, wherein the glucoamylase is present and/or added in saccharification and/or fermentation in combination with an alpha-amylase.
   42. The process of paragraph 41, wherein the alpha-amylase is present and/or added in saccharification and/or fermentation is of fungal or bacterial origin.
   43. The process of paragraph 41 or 42, wherein the alpha-amylase present and/or added in saccharification and/or fermentation is derived from a strain of the genus Rhizomucor, preferably a strain the Rhizomucor pusillus, such as the one shown in SEQ ID NO: 3 in WO 2013/006756, such as a Rhizomucor pusillus alpha-amylase hybrid having an Aspergillus niger linker and starch-bonding domain, such as the one shown in SEQ ID NO: 16 herein.
   44. The process of any of paragraphs 41-43, wherein the alpha-amylase present and/or added in saccharification and/or fermentation is selected from the group consisting of:
   (i) an alpha-amylase comprising the mature polypeptide of SEQ ID NO: 16 herein;
   (ii) an alpha-amylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the mature polypeptide of SEQ ID NO: 16 herein.
   45. The process of any of paragraphs 41-44, wherein the alpha-amylase is a variant of the alpha-amylase shown in SEQ ID NO: 16 having at least one of the following substitutions or combinations of substitutions: D165M; Y141W; Y141R; K136F; K192R; P224A; P224R; S123H+Y141W; G20S+Y141W; A76G+Y141W; G128D+Y141W; G128D+D143N; P219C+Y141W; N142D+D143N; Y141W+K192R; Y141W+D143N; Y141W+N383R; Y141W+P219C+A265C; Y141W+N142D+D143N; Y141W+K192R V410A; G128D+Y141W+D143N; Y141W+D143N+P219C; Y141W+D143N+K192R; G128D+D143N+K192R; Y141W+D143N+K192R+P219C; G128D+Y141W+D143N+K192R; or G128D+Y141W+D143N+K192R+P219C (using SEQ ID NO: 16 for numbering).
   46. The process of any of paragraphs 41-45, wherein the alpha-amylase is derived from a Rhizomucor pusillus with an Aspergillus niger glucoamylase linker and starch-binding domain (SBD), preferably disclosed as SEQ ID NO: 16 herein, preferably having one or more of the following substitutions: G128D, D143N, preferably G128D+D143N (using SEQ ID NO: 16 for numering).
   47. The process of any of paragraphs 41-46, wherein the alpha-amylase variant has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the mature part of the polypeptide of SEQ ID NO: 16 herein.
   48. The process of any of paragraphs 1-47, wherein liquefaction step i) is carried out using:
an alpha-amylase;
a protease having a thermostability value of more than 20% determined as Relative Activity at 80° C./70° C.; and
optionally a glucoamylase.
   49. The process of paragraph 48, wherein the protease with a thermostability value of more than 25% determined as Relative Activity at 80° C./70° C.
   50. The process of paragraphs 48-49, wherein the protease has a thermostability of more than 30%, more than 40%, more than 50%, more than 60%, more than 70%, more than 80%, more than 90%, more than 100%, such as more than 105%, such as more than 110%, such as more than 115%, such as more than 120% determined as Relative Activity at 80° C./70° C.
   51. The process of any of paragraphs 48-50, wherein the protease has a thermostability of between 20 and 50%, such as between 20 and 40%, such as 20 and 30% determined as Relative Activity at 80° C./70° C.
   52. The process of any of paragraphs 48-51, wherein the protease has a thermostability between 50 and 115%, such as between 50 and 70%, such as between 50 and 60%, such as between 100 and 120%, such as between 105 and 115% determined as Relative Activity at 80° C./70° C.
   53. The process of any of paragraphs 48-52, wherein the protease has a thermostability of more than 10%, such as more than 12%, more than 14%, more than 16%, more than 18%, more than 20%, more than 30%, more than 40%, more that 50%, more than 60%, more than 70%, more than 80%, more than 90%, more than 100%, more than 110% determined as Relative Activity at 85° C./70° C.
   54. The process of any of paragraphs 48-53, wherein the protease has thermostability of between 10 and 50%, such as between 10 and 30%, such as between 10 and 25% determined as Relative Activity at 85° C./70° C.
   55. The process of any of paragraphs 48-54, wherein the protease has a themostability above 60%, such as above 90%, such as above 100%, such as above 110% at 85° C. as determined using the Zein-BCA assay.
   56. The process of any of paragraphs 48-55, wherein the protease has a themostability between 60-120, such as between 70-120%, such as between 80-120%, such as between 90-120%, such as between 100-120%, such as 110-120% at 85° C. as determined using the Zein-BCA assay.
   57. The process of any of paragraphs 48-56, wherein the protease is of fungal origin.
   58. The process of any of paragraphs 48-57, wherein the protease is a variant of the metallo protease derived from a strain of the genus Thermoascus, preferably a strain of Thermoascus aurantiacus, especially Thermoascus aurantiacus CGMCC No. 0670.
   59. The process of any of paragraphs 48-58, wherein the protease is a variant of the metallo protease disclosed as the mature part of SEQ ID NO: 2 disclosed in WO 2003/048353 or the mature part of SEQ ID NO: 1 in WO 2010/008841 or SEQ ID NO: 3 herein mutations selected from the group of:
S5*+D79L+S87P+A112P+D142L;
D79L+S87P+A112P+T124V+D142L;
S5*+N26R+D79L+S87P+A112P+D142L;
N26R+T46R+D79L+S87P+A112P+D142L;
T46R+D79L+S87P+T116V+D142L;
D79L+P81R+S87P+A112P+D142L;
A27K+D79L+S87P+A112P+T124V+D142L;
D79L+Y82F+S87P+A112P+T124V+D142L;
D79L+Y82F+S87P+A112P+T124V+D142L;
D79L+S87P+A112P+T124V+A126V+D142L;
D79L+S87P+A112P+D142L;
D79L+Y82F+S87P+A112P+D142L;
S38T+D79L+S87P+A112P+A126V+D142L;
D79L+Y82F+S87P+A112P+A126V+D142L;
A27K+D79L+S87P+A112P+A126V+D142L;
D79L+S87P+N98C+A112P+G135C+D142L;
D79L+S87P+A112P+D142L+T141C+M161C;
S36P+D79L+S87P+A112P+D142L;
A37P+D79L+S87P+A112P+D142L;
S49P+D79L+S87P+A112P+D142L;
S50P+D79L+S87P+A112P+D142L;
D79L+S87P+D104P+A112P+D142L;
D79L+Y82F+S87G+A112P+D142L;
S70V+D79L+Y82F+S87G+Y97W+A112P+D142L;
D79L+Y82F+S87G+Y97W+D104P+A112P+D142L;
S70V+D79L+Y82F+S87G+A112P+D142L;
D79L+Y82F+S87G+D104P+A112P+D142L;
D79L+Y82F+S87G+A112P+A126V+D142L;
Y82F+S87G+S70V+D79L+D104P+A112P+D142L;
Y82F+S87G+D79L+D104P+A112P+A126V+D142L;
A27K+D79L+Y82F+S87G+D104P+A112P+A126V+D142L;
A27K+Y82F+S87G+D104P+A112P+A126V+D142L;
A27K+D79L+Y82F+D104P+A112P+A126V+D142L;
A27K+Y82F+D104P+A112P+A126V+D142L;
A27K+D79L+S87P+A112P+D142L; and
D79L+S87P+D142L.
   60. The process of any of paragraphs 48-59, wherein the protease is a variant of the metallo protease disclosed as the mature part of SEQ ID NO: 2 disclosed in WO 2003/048353 or the mature part of SEQ ID NO: 1 in WO 2010/008841 or SEQ ID NO: 3 herein with the following mutations:

### D79L+S87P+A112P+D142L:

### D79L+S87P+D142L; or

### A27K+D79L+Y82F+S87G+D104P+A112P+A126V+D142L.

61. The process of any of paragraphs 48-60, wherein the protease variant has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the mature part of the polypeptide of SEQ ID NO: 2 disclosed in WO 2003/048353 or the mature part of SEQ ID NO: 1 in WO 2010/008841 or SEQ ID NO: 3 herein.
62. The process of any of paragraphs 48-61, wherein the protease variant of the Thermoascus aurantiacus protease shown in SEQ ID NO: 3 herein is one of the following:
D79L S87P D142L
D79L S87P A112P D142L
D79L Y82F S87P A112P D142L
S38T D79L S87P A112P A126V D142L
D79L Y82F S87P A112P A126V D142L
A27K D79L S87P A112P A126V D142L
S49P D79L S87P A112P D142L
S50P D79L S87P A112P D142L
D79L S87P D104P A112P D142L
D79L Y82F S87G A112P D142L
S70V D79L Y82F S87G Y97W A112P D142L
D79L Y82F S87G Y97W D104P A112P D142L
S70V D79L Y82F S87G A112P D142L
D79L Y82F S87G D104P A112P D142L
D79L Y82F S87G A112P A126V D142L
Y82F S87G S70V D79L D104P A112P D142L
Y82F S87G D79L D104P A112P A126V D142L
A27K D79L Y82F S87G D104P A112P A126V D142L
   63. The process of any of paragraphs 48-62, wherein the protease is of bacterial origin.
   64. The process of any of paragraphs 48-63, wherein the protease is derived from a strain of Pyrococcus, preferably a strain of Pyrococcus furiosus.
   65. The process of any of paragraphs 1-64, wherein the protease is the one shown in SEQ ID NO: 1 in U.S. Pat. No. 6,358,726, or SEQ ID NO: 13 herein.
   66. The process of any of paragraphs 48-65, wherein the protease is one having at least 80%, such as at least 85%, such as at least 90%, such as at least 95%, such as at least 96%, such as at least 97%, such as at least 98%, such as at least 99% identity to in SEQ ID NO: 1 in U.S. Pat. No. 6,358,726 or SEQ ID NO: 13 herein.
   67. The process of any of paragraph 48-66, wherein 0.5-100 micro gram Pyrococcus furiosus protease per gram DS, such as 1-50 micro gram Pyrococcus furiosus protease per gram DS, such as 1-10 micro gram Pyrococcus furiosus protease per gram DS, such as 1.5-5 micro gram Pyrococcus furiosus protease per gram DS, such as around or more than 1.5 micro gram Pyrococcus furiosus protease per gram DS are present and/or added in liquefaction step i).
   68. The process of any of paragraphs 48-67, wherein 2-100 micro gram Pyrococcus furiosus protease per gram DS, such as 2.5-50 micro gram Pyrococcus furiosus protease per gram DS, such as 2.5-10 micro gram Pyrococcus furiosus protease per gram DS, such as 2.5-5 micro gram Pyrococcus furiosus protease gram DS, especially around 3 micro gram Pyrococcus furiosus protease per gram DS are present and/or added in liquefaction step i).
   69. The process of any of paragraphs 48-68, wherein a glucoamylase is present and/or added during liquefaction step i).
   70. The process of any of paragraphs 48-69, wherein the glucoamylase present and/or added in liquefaction has a heat stability at 85° C., pH 5.3, of at least 20%, such as at least 30%, preferably at least 35%.
   71. The process of any of paragraphs 48-70, wherein the glucoamylase present and/or added in liquefaction has a relative activity pH optimum at pH 5.0 of at least 90%, preferably at least 95%, preferably at least 97%.
   72. The process of any of paragraphs 48-71, wherein the glucoamylase present and/or added in liquefaction has a pH stability at pH 5.0 of at least at least 80%, at least 85%, at least 90%.
   73. The process of any of paragraphs 48-72, wherein the glucoamylase present and/or added in liquefaction step i) is derived from a strain of the genus Penicillium, especially a strain of Penicillium oxalicum disclosed as SEQ ID NO: 2 in WO 2011/127802 or SEQ ID NOs: 9 or 14 herein.
   74. The process of paragraph 48-73, wherein the glucoamylase has at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99% or 100% identity to the mature polypeptide shown in SEQ ID NO: 2 in WO 2011/127802 or SEQ ID NOs: 9 or 14 herein.
   75. The process of any of paragraphs 48-74, wherein the glucoamylase is a variant of the Penicillium oxalicum glucoamylase shown in SEQ ID NO: 2 in WO 2011/127802 having a K79V substitution (using the mature sequence shown in SEQ ID NO: 14 herein for numbering), such as a variant disclosed in WO 2013/053801.
   76. The process of any of paragraphs 48-75, wherein the Penicillium oxalicum glucoamylase has a K79V substitution (using SEQ ID NO: 14 herein for numbering) and further one of the following:

### T65A; or

### Q327F; or

### E501V; or

### Y504T; or

### Y504*; or

### T65A+Q327F; or

### T65A+E501V; or

### T65A+Y504T; or

### T65A+Y504*; or

### Q327F+E501V; or

### Q327F+Y504T; or

### Q327F+Y504*; or

### E501V+Y504T; or

### E501V+Y504*; or

### T65A+Q327F+E501V; or

### T65A+Q327F+Y504T; or

### T65A+E501V+Y504T; or

### Q327F+E501V+Y504T; or

### T65A+Q327F+Y504*; or

### T65A+E501V+Y504*; or

### Q327F+E501V+Y504*; or

### T65A+Q327F+E501V+Y504T; or

### T65A+Q327F+E501V+Y504*;

### E501V+Y504T; or

### T65A+K161S; or

### T65A+Q405T; or

### T65A+Q327W; or

### T65A+Q327F; or

### T65A+Q327Y; or

### P11F+T65A+Q327F; or

### R1K+D3W+K5Q+G7V+N8S+T10K+P11S+T65A+Q327F; or

### P2N+P4S+P11F+T65A+Q327F; or

### P11F+D26C+K33C+T65A+Q327F; or

### P2N+P4S+P11F+T65A+Q327W+E501V+Y504T; or

### R1E+D3N+P4G+G6R+G7A+N8A+T10D+P11D+T65A+Q327F; or

### P11F+T65A+Q327W; or

### P2N+P4S+P11F+T65A+Q327F+E501V+Y504T; or

### P11F+T65A+Q327W+E501V+Y504T; or

### T65A+Q327F+E501V+Y504T; or

### T65A+S105P+Q327W; or

### T65A+S105P+Q327F; or

### T65A+Q327W+S364P; or

### T65A+Q327F+S364P; or

### T65A+S103N+Q327F; or

### P2N+P4S+P11F+K34Y+T65A+Q327F; or

### P2N+P4S+P11F+T65A+Q327F+D445N+V447S; or

### P2N+P4S+P11F+T65A+I172V+Q327F; or

### P2N+P4S+P11F+T65A+Q327F+N502*; or

### P2N+P4S+P11F+T65A+Q327F+N502T+P563S+K571E; or

### P2N+P4S+P11F+R31S+K33V+T65A+Q327F+N564D+K571S; or

### P2N+P4S+P11F+T65A+Q327F+S377T; or

### P2N+P4S+P11F+T65A+V325T+Q327W; or

### P2N+P4S+P11F+T65A+Q327F+D445N+V447S+E501V+Y504T; or

### P2N+P4S+P11F+T65A+I172V+Q327F+E501V+Y504T; or

### P2N+P4S+P11F+T65A+Q327F+S377T+E501V+Y504T; or

### P2N+P4S+P11F+D26N+K34Y+T65A+Q327F; or

### P2N+P4S+P11F+T65A+Q327F+I375A+E501V+Y504T; or

### P2N+P4S+P11F+T65A+K218A+K221D+Q327F+E501V+Y504T; or

### P2N+P4S+P11F+T65A+S103N+Q327F+E501V+Y504T; or

### P2N+P4S+T10D+T65A+Q327F+E501V+Y504T; or

### P2N+P4S+F12Y+T65A+Q327F+E501V+Y504T; or

### K5A+P11F+T65A+Q327F+E501V+Y504T; or

### P2N+P4S+T10E+E18N+T65A+Q327F+E501V+Y504T; or

### P2N+T10E+E18N+T65A+Q327F+E501V+Y504T; or

### P2N+P4S+P11F+T65A+Q327F+E501V+Y504T+T568N; or

### P2N+P4S+P11F+T65A+Q327F+E501V+Y504T+K524T+G526A; or

### P2N+P4S+P11F+K34Y+T65A+Q327F+D445N+V447S+E501V+Y504T; or

### P2N+P4S+P11F+R31S+K33V+T65A+Q327F+D445N+V447S+E501V+Y504T; or

### P2N+P4S+P11F+D26N+K34Y+T65A+Q327F+E501V+Y504T; or

### P2N+P4S+P11F+T65A+F80*+Q327F+E501V+Y504T; or

### P2N+P4S+P11F+T65A+K112S+Q327F+E501V+Y504T; or

### P2N+P4S+P11F+T65A+Q327F+E501V+Y504T+T516P+K524T+G526A; or

### P2N+P4S+P11F+T65A+Q327F+E501V+N502T+Y504*; or

### P2N+P4S+P11F+T65A+Q327F+E501V+Y504T; or

### P2N+P4S+P11F+T65A+S103N+Q327F+E501V+Y504T; or

### K5A+P11F+T65A+Q327F+E501V+Y504T; or

### P2N+P4S+P11F+T65A+Q327F+E501V+Y504T+T516P+K524T+G526A; or

### P2N+P4S+P11F+T65A+K79A+Q327F+E501V+Y504T; or

### P2N+P4S+P11F+T65A+K79G+Q327F+E501V+Y504T; or

### P2N+P4S+P11F+T65A+K791+Q327F+E501V+Y504T; or

### P2N+P4S+P11F+T65A+K79L+Q327F+E501V+Y504T; or

### P2N+P4S+P11F+T65A+K79S+Q327F+E501V+Y504T; or

### P2N+P4S+P11F+T65A+L72V+Q327F+E501V+Y504T; or

### S255N+Q327F+E501V+Y504T; or

### P2N+P4S+P11F+T65A+E74N+V79K+Q327F+E501V+Y504T; or

### P2N+P4S+P11F+T65A+G220N+Q327F+E501V+Y504T; or

### P2N+P4S+P11F+T65A+Y245N+Q327F+E501V+Y504T; or

### P2N+P4S+P11F+T65A+Q253N+Q327F+E501V+Y504T; or

### P2N+P4S+P11F+T65A+D279N+Q327F+E501V+Y504T; or

### P2N+P4S+P11F+T65A+Q327F+S359N+E501V+Y504T; or

### P2N+P4S+P11F+T65A+Q327F+D370N+E501V+Y504T; or

### P2N+P4S+P11F+T65A+Q327F+V460S+E501V+Y504T; or

### P2N+P4S+P11F+T65A+Q327F+V460T+P468T+E501V+Y504T; or

### P2N+P4S+P11F+T65A+Q327F+T463N+E501V+Y504T; or

### P2N+P4S+P11F+T65A+Q327F+S465N+E501V+Y504T; or

### P2N+P4S+P11F+T65A+Q327F+T477N+E501V+Y504T.

77. The process of any of paragraphs 48-76, wherein the glucoamylase present and/or added in liquefaction is the Penicillium oxalicum glucoamylase has a K79V substitution (using SEQ ID NO: 14 herein for numbering) and further one of the following:
P11F+T65A+Q327F;
P2N+P4S+P11F+T65A+Q327F (using SEQ ID NO: 14 herein for numbering).
   78. The process of any of paragraphs 48-77, wherein the glucoamylase variant has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the mature part of the polypeptide of SEQ ID NO: 14 herein.
   79. The process of any of paragraphs 1-78, further wherein a pullulanase is present during liquefaction and/or saccharification.
   80. The process of any of paragraphs 1-79, comprising the steps of:
   i) liquefying the starch-containing material at a temperature above the initial gelatinization temperature using an alpha-amylase derived from Bacillus stearothermophilus;
   ii) saccharifying using a glucoamylase;
   iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.
   81. The process of any of paragraphs 1-80, comprising the steps of:
   i) liquefying the starch-containing material at a temperature above the initial gelatinization temperature using:
an alpha-amylase derived from Bacillus stearothermophilus comprising a double deletion at positions I181+G182, and optionally a N193F substitution; (using SEQ ID NO: 1 herein for numbering);
   ii) saccharifying using a glucoamylase derived from a strain of Gloephyllum, such as Gloephyllum serpiarium or Gloephyllum trabeum.
   iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.
   82. The process of any of paragraphs 1-81, comprising the steps of:
   i) liquefying the starch-containing material at a temperature above the initial gelatinization temperature using:
an alpha-amylase derived from Bacillus stearothermophilus;
a protease having a thermostability value of more than 20% determined as Relative Activity at 80° C./70° C., preferably derived from Pyrococcus furiosus and/or Thermoascus aurantiacus; and
optionally a Penicillium oxalicum glucoamylase;
   ii) saccharifying using a glucoamylase;
   iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.
   83. A process of paragraphs 1-82, comprising the steps of:
   i) liquefying the starch-containing material at a temperature above the initial gelatinization temperature using:
an alpha-amylase, preferably derived from Bacillus stearothermophilus, comprising a double deletion at positions I181+G182, and optionally a N193F substitution (using SEQ ID NO: 1 for numbering) and having a T½ (min) at pH 4.5, 85° C., 0.12 mM CaCl₂ of at least 10;
   ii) saccharifying using a glucoamylase;
   iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.
   84. A process of paragraphs 1-83, comprising the steps of:
i) liquefying the starch-containing material at a temperature between 80-90° C.:
   an alpha-amylase, preferably derived from Bacillus stearothermophilus, having a T½ (min) at pH 4.5, 85° C., 0.12 mM CaCl₂ of at least 10;
   a protease, preferably derived from Pyrococcus furiosus and/or Thermoascus aurantiacus, having a thermostability value of more than 20% determined as Relative Activity at 80° C./70° C.;
   optionally a Penicillium oxalicum glucoamylase
ii) saccharifying using a glucoamylase;
iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.
   85. A process of paragraphs 1-84, comprising the steps of:
   i) liquefying the starch-containing material at a temperature above the initial gelatinization temperature using:
an alpha-amylase derived from Bacillus stearothermophilus having a double deletion at positions I181+G182, and optional substitution N193F; and optionally further one of the following set of substitutions:
E129V+K177L+R179E;
V59A+Q89R+E129V+K177L+R179E+H208Y+K220P+N224L+Q254S:
V59A+Q89R+E129V+K177L+R179E+Q254S+M284V;
V59A+E129V+K177L+R179E+Q254S+M284V;
E129V+K177L+R179E+K220P+N224L+S242Q+Q254S (using SEQ ID NO: 1 herein for numbering);
ii) saccharifying using a glucoamylase, such as one from a strain of Gloephyllum, such as a strain of Gloephyllum serpiarium;
iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.
   86. A process of paragraphs 1-85, comprising the steps of:
   i) liquefying the starch-containing material at a temperature above the initial gelatinization temperature using:
an alpha-amylase derived from Bacillus stearothermophilus having a double deletion at positions I181+G182, and optional substitution N193F, and optionally further one of the following set of substitutions:
E129V+K177L+R179E;
V59A+Q89R+E129V+K177L+R179E+H208Y+K220P+N224L+Q254S:
V59A+Q89R+E129V+K177L+R179E+Q254S+M284V;
V59A+E129V+K177L+R179E+Q254S+M284V;
E129V+K177L+R179E+K220P+N224L+S242Q+Q254S (using SEQ ID NO: 1 herein for numbering).
a protease having a thermostability value of more than 20% determined as Relative Activity at 80° C./70° C., preferably derived from Pyrococcus furiosus and/or Thermoascus aurantiacus; and
optionally a Penicillium oxalicum glucoamylase shown in SEQ ID NO: 14 having substitutions selected from the group of:
K79V;
K79V+P11F+T65A+Q327F; or
K79V+P2N+P4S+P11F+T65A+Q327F; or
K79V+P11F+D26C+K33C+T65A+Q327F; or
K79V+P2N+P4S+P11F+T65A+Q327W+E501V+Y504T; or
K79V+P2N+P4S+P11F+T65A+Q327F+E501V+Y504T; or
K79V+P11F+T65A+Q327W+E501V+Y504T (using SEQ ID NO: 14 for numbering);
ii) saccharifying using a glucoamylase;
iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.
   87. A process of paragraphs 1-86, comprising the steps of:
   i) liquefying the starch-containing material at a temperature between 80-90° C. using:
an alpha-amylase derived from Bacillus stearothermophilus having a double deletion at positions I181+G182, and optional substitution N193F, and further optionally one of the following set of substitutions:
E129V+K177L+R179E;
V59A+Q89R+E129V+K177L+R179E+H208Y+K220P+N224L+Q254S;
V59A+Q89R+E129V+K177L+R179E+Q254S+M284V;
V59A+E129V+K177L+R179E+Q254S+M284V;
E129V+K177L+R179E+K220P+N224L+S242Q+Q254S (using SEQ ID NO: 1 herein for numbering),
a protease having a thermostability value of more than 20% determined as Relative Activity at 80° C./70° C., preferably derived from Pyrococcus furiosus and/or Thermoascus aurantiacus;
a Penicillium oxalicum glucoamylase shown in SEQ ID NO: 14 having substitutions selected from the group of:
K79V;
K79V+P11F+T65A+Q327F; or
K79V+P2N+P4S+P11F+T65A+Q327F; or
K79V+P11F+D26C+K33C+T65A+Q327F; or
K79V+P2N+P4S+P11F+T65A+Q327W+E501V+Y504T; or
K79V+P2N+P4S+P11F+T65A+Q327F+E501V+Y504T; or
K79V+P11F+T65A+Q327W+E501V+Y504T (using SEQ ID NO: 14 for numbering);
ii) saccharifying using a glucoamylase;
iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.
   88. The process of any of paragraphs 1-87, comprising the steps of:
   i) liquefying the starch-containing material at a temperature above the initial gelatinization temperature using:
an alpha-amylase derived from Bacillus stearothermophilus having a double deletion at positions I181+G182, and optional substitution N193F (using SEQ ID NO: 1 herein for numbering);
a protease having a thermostability value of more than 20% determined as Relative Activity at 80° C./70° C., preferably derived from Pyrococcus furiosus and/or Thermoascus aurantiacus; and
optionally a pullulanase;
a Penicillium oxalicum glucoamylase having a K79V substilution (using SEQ ID NO: 14 herein for numbering);
   ii) saccharifying using a glucoamylase;
   iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.
   89. A process of paragraphs 1-88, comprising the steps of:
i) liquefying the starch-containing material at a temperature above the initial gelatinization temperature using:
an alpha-amylase, preferably derived from Bacillus stearothermophilus, having a T½ (min) at pH 4.5, 85° C., 0.12 mM CaCl₂ of at least 10;
between 0.5 and 10 micro grams Pyrococcus furiosus protease per g DS;
ii) saccharifying using a glucoamylase selected from the group of glucoamylase derived from a strain of Aspergillus, preferably A. niger, A. awamori, or A. oryzae; or a strain of Trichoderma, preferably T. reesei; or a strain of Talaromyces, preferably T. emersonii, or a strain of Pycnoporus, or a strain of Gloephyllum, such as G. serpiarium or G. trabeum, or a strain of the Nigrofomes;
iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.
   90. A process of paragraphs 1-89, comprising the steps of:
i) liquefying the starch-containing material at a temperature between 80-90° C. using;
   an alpha-amylase, preferably derived from Bacillus stearothermophilus having a double deletion at positions I181+G182, and optional substitution N193F and having a T½ (min) at pH 4.5, 85° C., 0.12 mM CaCl₂ of at least 10;
   between 0.5 and 10 micro grams Pyrococcus furiosus protease per g DS;
   optionally a pullulanase;
   a Penicillium oxalicum glucoamylase;
ii) saccharifying using a glucoamylase;
iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.
   91. A process of paragraphs 1-90, comprising the steps of:
i) liquefying the starch-containing material at a temperature a temperature between 80-90° C. using;
   an alpha-amylase derived from Bacillus stearothermophilus having a double deletion I181+G182 and optional substitution N193F; and optionally further one of the following set of substitutions:
   E129V+K177L+R179E;
   V59A+Q89R+E129V+K177L+R179E+H208Y+K220P+N224L+Q254S;
   V59A+Q89R+E129V+K177L+R179E+Q254S+M284V:
   V59A+E129V+K177L+R179E+Q254S+M284V
   E129V+K177L+R179E+K220P+N224L+S242Q+Q254S (using SEQ ID NO: 1 herein for numbering);
   between 0.5 and 10 micro grams Pyrococcus furiosus protease per g DS; and
   optionally a pullulanase;
   a Penicillium oxalicum glucoamylase shown in SEQ ID NO: 14 having substitutions selected from the group of:
   K79V;
   K79V+P11F+T65A+Q327F; or
   K79V+P2N+P4S+P11F+T65A+Q327F; or
   K79V+P11F+D26C+K33C+T65A+Q327F; or
   K79V+P2N+P4S+P11F+T65A+Q327W+E501V+Y504T; or
   K79V+P2N+P4S+P11F+T65A+Q327F+E501V+Y504T; or
   K79V+P11F+T65A+Q327W+E501V+Y504T (using SEQ ID NO: 14 for numbering);
ii) saccharifying using a glucoamylase;
iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.
   92. A process of paragraphs 1-91, comprising the steps of:
i) liquefying the starch-containing material at a temperature between 80-90° C. using:
   an alpha-amylase derived from Bacillus stearothermophilus having a double deletion I181+G182 and optional substitution N193F; and further one of the following set of substitutions:
   E129V+K177L+R179E;
   V59A+Q89R+E129V+K177L+R179E+H208Y+K220P+N224L+Q254S;
   V59A+Q89R+E129V+K177L+R179E+Q254S+M284V;
   V59A+E129V+K177L+R179E+Q254S+M284V
   E129V+K177L+R179E+K220P+N224L+S242Q+Q254S (using SEQ ID NO: 1 herein for numbering).
   a protease having a thermostability value of more than 20% determined as Relative Activity at 80° C./70° C., preferably derived from Pyrococcus furiosus and/or Thermoascus aurantiacus; and
   optionally a pullulanase;
   a Penicillium oxalicum glucoamylase shown in SEQ ID NO: 14 having substitutions selected from the group of:
   K79V;
   K79V+P11F+T65A+Q327F; or
   K79V+P2N+P4S+P11F+T65A+Q327F; or
   K79V+P11F+D26C+K33C+T65A+Q327F; or
   K79V+P2N+P4S+P11F+T65A+Q327W+E501V+Y504T; or
   K79V+P2N+P4S+P11F+T65A+Q327F+E501V+Y504T; or
   K79V+P11F+T65A+Q327W+E501V+Y504T (using SEQ ID NO: 14 for numbering);
ii) saccharifying using a glucoamylase selected from the group of glucoamylase derived from a strain of Aspergillus; or a strain of Trichoderma; a strain of Talaromyces, a strain of Pycnoporus; a strain of Gloephyllum; and a strain of the Nigrofomes;
iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.
   93. A process of any of paragraphs 1-92, comprising the steps of:
i) liquefying the starch-containing material at a temperature between 80-90° C. at a pH between 5.0 and 6.5 using:
   an alpha-amylase derived from Bacillus stearothermophilus having a double deletion I181+G182 and optional substitution N193F; and optionally further one of the following set of substitutions:
   E129V+K177L+R179E;
   V59A+Q89R+E129V+K177L+R179E+H208Y+K220P+N224L+Q254S;
   V59A+Q89R+E129V+K177L+R179E+Q254S+M284V;
   V59A+E129V+K177L+R179E+Q254S+M284V
   E129V+K177L+R179E+K220P+N224L+S242Q+Q254S (using SEQ ID NO: 1 herein for numbering).
   a protease derived from Pyrococcus furiosus, preferably the one shown in SEQ ID NO: 13 herein;
   a Penicillium oxalicum glucoamylase shown in SEQ ID NO: 14 having substitutions selected from the group of:
   K79V;
   K79V+P11F+T65A+Q327F; or
   K79V+P2N+P4S+P11F+T65A+Q327F; or
   K79V+P11F+D26C+K33C+T65A+Q327F; or
   K79V+P2N+P4S+P11F+T65A+Q327W+E501V+Y504T; or
   K79V+P2N+P4S+P11F+T65A+Q327F+E501V+Y504T; or
   K79V+P11F+T65A+Q327W+E501V+Y504T (using SEQ ID NO: 14 for numbering);
ii) saccharifying using a glucoamylase;
iii) fermenting using a fermenting organism;
   wherein an acid having a pKa in the range from 3.75 to 5.75 is present or added in fermentation so that the acid concentration in fermentation is maintained between above 0 (zero) and 100 mmoles/L fermentation medium and wherein the acid is added before the exponential growth phase of the fermenting organism.
   94. The process of any of paragraphs 1-93, wherein a cellulolytic composition is present in saccharification, fermentation or simultaneous saccharification and fermentation (SSF).
   95. The process of any of paragraphs 1-94, wherein the acid concentration is maintained between 10 and 100 mmoles/L fermentation medium.
   96. The process of any of paragraphs 1-95, wherein the acid concentration is maintained between 5 and 80 mmoles/L fermentation medium.
   97. A process for producing a fermentation product from starch-containing material comprising the steps of:
   (i) saccharifying the starch-containing material at a temperature below the initial gelatinization temperature
   (ii) fermenting using a fermenting organism;
wherein saccharification and/or fermentation is done in the presence of the following enzymes: glucoamylase and alpha-amylase, and optionally protease; and wherein an acid having a pKa in the range from 3.75 to 5.75 is present and/or added in fermentation so that the acid concentration in fermentation is maintained between above 0 (zero) and 100 mmoles/L fermentation medium and wherein the acid is added before the exponential growth phase of the fermenting organism.
   98. The process of paragraph 97, wherein the acid concentration is maintained between 10 and 100 mmoles/L fermentation medium.
   99. The process of paragraphs 97 or 98, wherein the acid concentration is maintained between 5 and 80 mmoles/L fermentation medium.
   100. The process of any of paragraphs 97-99, wherein saccharification and fermentation is carried out simultaneosly (one step process).
   101. The process of any of paragraphs 97-100, wherein the glucoamylase is a Gloeophyllum glucoamylase, preferably Gloeophyllum trabeum glucoamylase.
   102. The process of any of paragraphs 97-101, wherein the glucoamylase is the Gloeophyllum trabeum glucoamylase shown in SEQ ID NO: 17 herein.
   103. The process of any of paragraphs 98-102, wherein the glucoamylase is the Gloeophyllum trabeum glucoamylase shown in SEQ ID NO: 17 having one of the following substitutions: V59A; S95P; A121P; T119W; S95P+A121P; V59A+S95P; S95P+T119W; V59A+S95P+A121P; or S95P+T119W+A121P, especially S95P+A121P.
   104. The process of any of paragraphs 97-103, wherein the glucoamylase is a Trametes glucoamylase, preferably Trametes cingulata glucoamylase.
   105. The process of any of paragraphs 97-104, wherein the glucoamylase is the Trametes cingulata glucoamylase shown in SEQ ID NO: 20 herein.
   106. The process of any of paragraphs 97-105, wherein the glucoamylase is selected from the group consisting of:
   (i) a glucoamylase comprising the mature polypeptide of SEQ ID NO: 20 herein;
   (ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the mature polypeptide of SEQ ID NO: 20 herein.
   107. The process of any of paragraphs 97-106, wherein the alpha-amylase is derived from Rhizomucor pusillus, preferably with an Aspergillus niger glucoamylase linker and starch-binding domain (SBD), preferably the one disclosed as V039 in Table 5 in WO 2006/069290 or SEQ ID NO: 16 herein.
   108. The process any of paragraphs 97-107, wherein the glucoamylase is the Trametes cingulata glucoamylase shown in SEQ ID NO: 20 and the alpha-amylase is Rhizomucor pusillus alpha-amylase with an Aspergillus niger glucoamylase linker and starch-binding domain (SBD).
   109. The process of any of paragraphs 97-108, wherein the alpha-amylase is derived from Rhizomucor pusillus.
   110. The process of any of paragraphs 97-109, wherein the glucoamylase, such as one derived from Gloeophyllum trabeum, is selected from the group consisting of:
   (i) a glucoamylase comprising the mature polypeptide of SEQ ID NO: 17 herein;
   (ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the mature polypeptide of SEQ ID NO: 17 herein.
   111. The process of any of paragraphs 97-110, wherein the alpha-amylase is Rhizomucor pusillus alpha-amylase with an Aspergillus niger glucoamylase linker and starch-binding domain (SBD), preferably one having at least one of the following substitutions or combinations of substitutions: D165M; Y141W; Y141R; K136F; K192R; P224A; P224R; S123H+Y141W; G20S+Y141W; A76G+Y141W; G128D+Y141W; G128D+D143N; P219C+Y141W; N142D+D143N; Y141W+K192R; Y141W+D143N; Y141W+N383R; Y141W+P219C+A265C; Y141W+N142D+D143N; Y141W+K192R V410A; G128D+Y141W+D143N; Y141W+D143N+P219C; Y141W+D143N+K192R; G128D+D143N+K192R; Y141W+D143N+K192R+P219C; G128D+Y141W+D143N+K192R; or G128D+Y141W+D143N+K192R+P219C, especially G128D+D143N (using SEQ ID NO: 16 herein for numbering).
   112. The process any of paragraphs 97-111, wherein the glucoamylase is the Gloeophyllum trabeum glucoamylase shown in SEQ ID NO: 17 herein having one of the following substitutions: S95P+A121P and the alpha-amylase is Rhizomucor pusillus alpha-amylase with an Aspergillus niger glucoamylase linker and starch-binding domain (SBD), preferably one having the following substitutions G128D+D143N (using SEQ ID NO: 16 herein for numbering).
   113. The process of any of paragraphs 97-112, wherein the glucoamylase is the Pycnoporus sanguineus glucoamylase shown in SEQ ID NO: 18 herein.
   114. The process of any of paragraphs 97-113, wherein the glucoamylase, such as one from Pycnoporus sanguineus, is selected from the group consisting of:
   (i) a glucoamylase comprising the mature polypeptide of SEQ ID NO: 18 herein;
   (ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the mature polypeptide of SEQ ID NO: 18 herein.
   115. The process of any of paragraphs 97-114, wherein the glucoamylase is the Pycnoporus sanguineus glucoamylase shown in SEQ ID NO: 18 herein, and the alpha-amylase is the Rhizomucor pusillus with an Aspergillus niger glucoamylase linker and starch-binding domain (SBD), preferably the one disclosed as V039 in Table 5 in WO 2006/069290 or SEQ ID NO: 16 herein, preferably one having one or more of the following substitutions: G128D, D143N, especially G128D+D143N.
   116. The process of any of paragraphs 97-115, wherein the ratio between glucoamylase and alpha-amylase is between 99:1 and 1:2, such as between 98:2 and 1:1, such as between 97:3 and 2:1, such as between 96:4 and 3:1, such as 97:3, 96:4, 95:5, 94:6, 93:7, 90:10, 85:15, 83:17 or 65:35 (mg EP glucoamylase: mg EP alpha-amylase).
   117. The process of any of paragraphs 97-116, wherein the total dose of glucoamylase and alpha-amylase added is from 10-1,000 μg/g DS, such as from 50-500 μg/g DS, such as 75-250 μg/g DS.
   118. The process of any of paragraphs 97-117, wherein the total dose of cellulolytic enzyme composition added is from 10-500 μg/g DS, such as from 20-400 μg/g DS, such as 20-300 μg/g DS.
   119. The process of any of paragraphs 97-118, wherein the dose of protease added is from 1-200 μg/g DS, such as from 2-100 μg/g DS, such as 3-50 μg/g DS.

1. A process for producing a fermentation product from starch-containing material comprising the steps of:
i) liquefying the starch-containing material at a temperature above the initial gelatinization temperature using an alpha-amylase;
ii) saccharifying using a glucoamylase; and
iii) fermenting using a fermenting organism;
wherein an acid having a pKa in the range from 3.75 to 5.75 is present and/or added in fermentation so that the acid concentration in fermentation is maintained between above 0 (zero) and 100 mmoles/L fermentation medium and wherein the acid is added before the exponential growth phase of the fermenting organism.

2. The process of claim 1, wherein the fermenting organism is a yeast.

3. The process of claim 1, wherein the fermenting organism is a strain of Saccharomyces cerevisiae.

4. The process of claim 1, wherein the acid concentration in fermentation is maintained between 5 and 80 mmoles/L.

5. The process of claim 1, wherein the acid is added during lag phase.

6. The process of claim 1, wherein the acid has a pKa in the range from 4.0 to 5.0.

7. The process of claim 1, wherein the acid is selected from acetic acid, benzoic acid, propionic acid, formic acid, sorbic acid and succinic acid.

8. The process of claim 1, wherein the acid concentration in fermemntation is between 20-80 mmoles/L and the acid is acetic acid.

9. The process of claim 1, wherein the acid is hydrophobic when protonated.

10. The process of claim 1, wherein the fermentation product is ethanol.

11. The process of claim 1, wherein a nitrogen source, is added in saccharification, fermentation, or simultaneous saccharification and fermentation (SSF).

12. The process of claim 1,
wherein the alpha-amylase is derived from Bacillus stearothermophilus.

13. A process for producing a fermentation product from starch-containing material comprising the steps of:
(i) saccharifying the starch-containing material at a temperature below the initial gelatinization temperature; and
(ii) fermenting using a fermenting organism;
wherein saccharification and/or fermentation is done in the presence of the following enzymes: glucoamylase and alpha-amylase, and optionally protease; and wherein an acid having a pKa in the range from 3.75 to 5.75 is present and/or added in fermentation so that the acid concentration in fermentation is maintained between above 0 (zero) and 100 mmoles/L fermentation medium and wherein the acid is added before the exponential growth phase of the fermenting organism.

14. The process of claim 13, wherein the fermenting organism is yeast.

15. The process of claim 13, wherein the fermenting organism is a strain of Saccharomyces cerevisiae.

16. The process of claim 13, wherein the acid concentration in fermentation is maintained between 5 and 80 mmoles/L.

17. The process of claim 13, wherein the acid is added during lag phase.

18. The process of claim 13, wherein the acid has a pKa in the range from 4.0 to 5.0.

19. The process of claim 13, wherein the acid is selected from acetic acid, benzoic acid, propionic acid, formic acid, sorbic acid and succinic acid.

20. The process of claim 13, wherein the acid is hydrophobic when protonated.


SEQUENCE LISTING (28 sequences)

SEQ ID NO: 1 - protein, 515 aa, Bacillus stearothermophilus
  mat_peptide (1)..(515)
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 2 - DNA, 1068 bp, Thermoascus aurantiacus
  CDS (1)..(1065)
  misc_signal (1)..(57)
  misc_feature (58)..(534)
  mat_peptide (535)..(1068)
atgcggctcgttgcttccctaacggccttggtggccttgtccgtacctgtctttcccgctgctgtcaacgtgaagcgtgcttcgtcctacctggagatcactctgagccaggtcagcaacactctgatcaaggccgtggtccagaacactggtagcgacgagttgtccttcgttcacctgaacttcttcaaggaccccgctcctgtcaaaaaggtatcggtctatcgcgatgggtctgaagtgcagttcgagggcattttgagccgctacaaatcgactggcctctctcgtgacgcctttacttatctggctcccggagagtccgtcgaggacgtttttgatattgcttcgacttacgatctgaccagcggcggccctgtaactatccgtactgagggagttgttccctacgccacggctaacagcactgatattgccggctacatctcatactcgtctaatgtgttgaccattgatgtcgatggcgccgctgctgccactgtctccaaggcaatcactcctttggaccgccgcactaggatcagttcctgctccggcagcagacagagcgctcttactacggctctcagaaacgctgcttctcttgccaacgcagctgccgacgcggctcagtctggatcagcttcaaagttcagcgagtacttcaagactacttctagctctacccgccagaccgtggctgcgcgtcttcgggctgttgcgcgggaggcatcttcgtcttcttcgggagccaccacgtactactgcgacgatccctacggctactgttcctccaacgtcctggcttacaccctgccttcatacaacataatcgccaactgtgacattttctatacttacctgccggctctgaccagtacctgtcacgctcaggatcaagcgaccactgcccttcacgagttcacccatgcgcctggcgtctacagccctggcacggacgacctggcgtatggctaccaggctgcgatgggtctcagcagcagccaggctgtcatgaacgctgacacctacgctctctatgcgaatgccatataccttggttgctaa

SEQ ID NO: 3 - protein, 355 aa, Thermoascus aurantiacus
MRLVASLTALVALSVPVFPAAVNVKRASSYLEITLSQVSNTLIKAVVQNTGSDELSFVHLNFFKDPAPVKKVSVYRDGSEVQFEGILSRYKSTGLSRDAFTYLAPGESVEDVFDIASTYDLTSGGPVTIRTEGVVPYATANSTDIAGYISYSSNVLTIDVDGAAAATVSKAITPLDRRTRISSCSGSRQSALTTALRNAASLANAAADAAQSGSASKFSEYFKTTSSSTRQTVAARLRAVAREASSSSSGATTYYCDDPYGYCSSNVLAYTLPSYNIIANCDIFYTYLPALTSTCHAQDQATTALHEFTHAPGVYSPGTDDLAYGYQAAMGLSSSQAVMNADTYALYANAIYLGC

SEQ ID NO: 4 - DNA, 49 bp, Artificial Sequence
  Synthetic Construct
aacgacggtacccggggatcggatccatgcggctcgttgcttccctaac

SEQ ID NO: 5 - DNA, 48 bp, Artificial Sequence
  Artificial Construct
ctaattacatgatgcggcccttaattaattagcaaccaaggtatatgg

SEQ ID NO: 6 - DNA, 20 bp, Artificial Sequence
  Artificial Construct
taggagtttagtgaacttgc

SEQ ID NO: 7 - DNA, 18 bp, Artificial Sequence
  Artificial Construct
ttcgagcgtcccaaaacc

SEQ ID NO: 8 - DNA, 1851 bp, Penicillium oxalicum
  CDS (1)..(1851)
  misc_signal (1)..(63)
atgcgtctcactctattatcaggtgtagccggcgttctctgcgcaggacagctgacggcggcgcgtcctgatcccaagggtgggaatctgacgccgttcatccacaaagagggcgagcggtcgctccaaggcatcttggacaatctcggtgggcgaggtaagaaaacacccggcactgccgcagggttgtttattgccagtccaaacacagagaatccaaactattattatacatggactcgtgactcagctttgactgccaagtgcttgatcgacctgttcgaagactctcgggcaaagtttccaattgaccgcaaatacttggaaacaggaattcgggactacgtgtcgtcccaagcaatcctccagagtgtgtctaatccttctggaaccctgaaggatggctctggtctgggtgaacccaagtttgagattgacctgaatcccttttcgggtgcctggggtcggcctcagcgggatggcccagcgctgcgagcgaccgctatgatcacctacgccaactacctgatatcccatggtcagaaatcggatgtgtcacaggtcatgtggccgattattgccaatgatctagcatatgttggtcaatactggaataataccggatttgacctgtgggaagaggtggatgggtcaagctttttcacgattgcggtccagcaccgagcccttgttgaaggctcgcaactggcgaaaaagctcggcaagtcctgcgatgcctgtgattctcagcctccccagatattgtgtttcctgcagagtttctggaacggaaagtacatcacctccaacatcaacacgcaagcaagccgctctggtatcgacctggactctgtcctgggaagcattcatacctttgatcccgaagcagcctgtgacgatgcaactttccagccttgttctgcccgcgctctggcgaaccacaaggtctatgtggattccttccgctctatctacaagattaatgcgggtcttgcagagggatcggctgccaacgttggccgctaccccgaggatgtttaccaaggaggcaatccatggtatctcgccaccctaggcgcatctgaattgctttacgacgccttgtaccagtgggacagacttggcaaacttgaagtctcggagacctcgttgtcattcttcaaagactttgacgcgaccgtgaaaattggctcgtactcgaggaacagcaagacctacaagaaattgacccagtccatcaagtcgtacgcggacgggttcatccagttagtgcagcagtacactccttctaatggatctctggccgagcaatacgatcgcaatacggctgctcctctctctgcaaacgatctgacttggtcatttgcctctttcttgacggctacgcaacgccgcgatgccgtggttcctccctcctggggcgcaaagtcggcaaacaaagtcccaaccacttgttcagcctcccctgttgtgggtacttataaggcgcccacggcaactttctcatccaagactaagtgcgtccccgctaaagatattgtgcctatcacgttctacctgattgagaacacttactatggagagaacgtcttcatgagtggcaacattactgcgctgggtaactgggacgccaagaaaggcttcccactcaccgcaaacctctacacgcaagatcaaaacttgtggttcgccagtgtcgagttcatcccagcaggcacaccctttgagtacaagtactacaaggtcgagcccaatggcgatattacttgggagaagggtcccaaccgggtgttcgtcgctcccacgggatgcccagttcagcctcactccaacgacgtgtggcagttttga

SEQ ID NO: 9 - protein, 616 aa, Penicillium oxalicum
MRLTLLSGVAGVLCAGQLTAARPDPKGGNLTPFIHKEGERSLQGILDNLGGRGKKTPGTAAGLFIASPNTENPNYYYTWTRDSALTAKCLIDLFEDSRAKFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 10 - DNA, 4014 bp, Thermococcus hydrothermalis
  CDS (1)..(4011)
  misc_signal (1)..(81)
  mat_peptide (82)..(4014)
atgaggcgggtggttgccctcttcattgcaattttgatgcttggaagcatcgttggagcgaacgttaagagcgttggcgcggcggagccgaagccgctcaacgtcataatagtctggcaccagcaccagccctactactacgaccctgtccaggacgtctacaccaggccctgggtcaggctccacgcggcgaacaactactggaagatggcccactacctgagccagtacccggaggttcacgccaccattgacctctcgggttcgctgatagcccagcttgccgactacatgaacggcaagaaggacacctaccagataatcaccgagaagatagccaacggggaacccctcaccgtcgacgagaagtggttcatgctccaggcaccgggagggttcttcgacaacaccatcccctggaacggtgaaccgataaccgaccccaacggcaacccgataagggacttctgggaccgctacacggagctgaagaacaagatgctcagcgcaaaggccaagtacgcaaacttcgtgactgagagccagaaggtcgctgtgacgaacgagttcacagagcaggactacatagacctagcggttctcttcaatctcgcttggattgactacaattacatcacgagcacgccggagttcaaggccctctacgacaaggttgacgagggcggctatacaagggcggacgtcaaaaccgttctcgacgcccagatctggcttctcaaccacaccttcgaggagcacgagaagataaacctcctcctcggaaacggcaacgtcgaggtcacggtcgttccctacgcccacccgataggcccgatactcaacgacttcggctgggacagcgacttcaacgaccaggtcaagaaggccgacgaactgtacaagccgtacctcggcggcggcaccgcggttccaaaaggcggatgggcggctgagagcgccctcaacgacaaaactctggagatcctcgccgagaacggctgggagtgggtcatgaccgaccagatggttctcggaaagctcggcattgagggaaccgtcgagaactaccacaagccctgggtggccgagttcaacggaaagaagatatacctcttcccaagaaatcacgatctaagtgacagagttggctttacctacagcggaatgaaccagcagcaggccgttgaggacttcgtcaacgagctcctcaagctccagaagcagaactacgatggctcgctggtttacgtggtcacgctcgacggcgagaaccccgtggagaactacccctacgacggggagctcttcctcaccgaactctacaagaagctgaccgaactccaggagcagggtctcataagaaccctcaccccgagcgagtacatccagctctacggcgacaaggccaacaagctcacacctcggatgatggagcgccttgacctcaccggagacaacgttaacgccctcctcaaggcccagagcctcggcgaactctacgacatgaccggcgttaaggaggagatgcagtggcccgagagcagctggatagacggaaccctctccacgtggataggcgagccccaggagaactacggctggtactggctctacatggccaggaaggcccttatggagaacaaggataaaatgagccaggcggactgggagaaggcctacgagtacctgctccgcgccgaggcaagcgactggttctggtggtacggaagcgaccaggacagcggccaggactacaccttcgaccgctacctgaagacctacctctacgagatgtacaagctggcaggagtcgagccgccgagctacctcttcggcaactacttcccggacggagagccctacaccacgaggggcctggtcggactcaaggacggcgagatgaagaacttctccagcatgtccccgctggcaaagggcgtgagcgtctatttcgacggcgaggggatacacttcatagtgaaagggaacctggacaggttcgaggtgagcatctgggagaaggatgagcgcgttggcaacacgttcacccgcctccaagagaagccggacgagttgagctatttcatgttcccattctcaagggacagcgttggtctcctcataaccaagcacgtcgtgtacgagaacggaaaggccgagatatacggcgccaccgactacgagaagagcgagaagcttggggaagccaccgtcaagaacacgagcgaaggaatcgaagtcgtccttccctttgactacatagaaaacccctccgacttctacttcgctgtctcgacggtcaaagatggagaccttgaggtgataagcactcctgtggagctcaagctcccgaccgaggtcaagggagtcgtcatagccgatataaccgacccagaaggcgacgaccatgggcccggaaactacacttatcccacggacaaggtcttcaagccaggtgttttcgacctcctccgcttcaggatgctcgaacagacggagagctacgtcatggagttctacttcaaggacctaggtggtaacccgtggaacggacccaacggcttcagcctccagataatcgaggtctacctcgacttcaaggacggtggaaacagttcggccattaagatgttccccgacggaccgggagccaacgtcaacctcgaccccgagcatccatgggacgttgccttcaggatagcgggctgggactacggaaacctcatcatcctgccgaacggaacggccatccagggcgagatgcagatttccgcagatccggttaagaacgccataatagtcaaggttccaaagaagtacatcgccataaacgaggactacggcctctggggagacgtcctcgtcggctcgcaggacggctacggcccggacaagtggagaacggcggcagtggatgcggagcagtggaagcttggaggtgcggacccgcaggcagtcataaacggcgtggccccgcgcgtcattgatgagctggttccgcagggctttgaaccgacccaggaggagcagctgagcagctacgatgcaaacgacatgaagctcgccactgtcaaggcgctgctactcctcaagcagggcatcgttgtgaccgacccggagggagacgaccacgggccgggaacgtacacctatccgacggacaaagttttcaagcccggtgttttcgacctcctcaagttcaaggtgaccgagggaagcgacgactggacgctggagttccacttcaaagacctcggtggaaacccgtggaacgggccgaacggcttcagcctgcagataatcgaggtatacttcgacttcaaggagggcgggaacgtctcggccattaagatgttcccggatgggcccggaagcaacgtccgtcttgatccaaatcacccatgggacctggcgcttaggatagccggctgggactacggaaacctgataattctgcccgacggaaccgcctaccaaggcgagatgcagatttccgcagatccggttaagaacgccataatagtcaaggttccaaagaagtacctgaacatatccgactacggactctacaccgccgtcatcgtgggttcccaagacgggtacggcccggacaagtggaggcccgtggccgctgaggccgagcagtggaagctcggaggcgcagacccccaggcggtcatagacaacctcgtaccaagggtcgttgatgaactcgtgccggagggcttcaagccaacgcaggaggagcagctgagcagctacgaccttgagaagaagaccctggcgacggtgctcatggtaccgctcgtcaatgggactggcggcgaggaaccaacgccgacggagagcccaacggaaacgacgacaaccacacccagcgaaacaaccaccacaacttcaacgaccaccggcccaagctcaacgaccaccagcacacccggcggaggaatctgcggcccaggcattatagcgggcctggccctgataccgctcctcctcaagaggaggaactga

SEQ ID NO: 11 - protein, 1337 aa, Thermococcus hydrothermalis
MRRVVALFIAILMLGSIVGANVKSVGAAEPKPLNVIIVWHQHQPYYYDPVQDVYTRPWVRLHAANNYWKMAHYLSQYPEVHATIDLSGSLIAQLADYMNGKKDTYQIITEKIANGEPLTVDEKWFMLQAPGGFFDNTIPWNGEPITDPNGNPIRDFWDRYTELKNKMLSAKAKYANFVTESQKVAVTNEFTEQDYIDLAVLFNLAWIDYNYITSTPEFKALYDKVDEGGYTRADVKTVLDAQIWLLNHTFEEHEKINLLLGNGNVEVTVVPYAHPIGPILNDFGWDSDFNDQVKKADELYKPYLGGGTAVPKGGWAAESALNDKTLEILAENGWEWVMTDQMVLGKLGIEGTVENYHKPWVAEFNGKKIYLFPRNHDLSDRVGFTYSGMNQQQAVEDFVNELLKLQKQNYDGSLVYVVTLDGENPVENYPYDGELFLTELYKKLTELQEQGLIRTLTPSEYIQLYGDKANKLTPRMMERLDLTGDNVNALLKAQSLGELYDMTGVKEEMQWPESSWIDGTLSTWIGEPQENYGWYWLYMARKALMENKDKMSQADWEKAYEYLLRAEASDWFWWYGSDQDSGQDYTFDRYLKTYLYEMYKLAGVEPPSYLFGNYFPDGEPYTTRGLVGLKDGEMKNFSSMSPLAKGVSVYFDGEGIHFIVKGNLDRFEVSIWEKDERVGNTFTRLQEKPDELSYFMFPFSRDSVGLLITKHVVYENGKAEIYGATDYEKSEKLGEATVKNTSEGIEVVLPFDYIENPSDFYFAVSTVKDGDLEVISTPVELKLPTEVKGVVIADITDPEGDDHGPGNYTYPTDKVFKPGVFDLLRFRMLEQTESYVMEFYFKDLGGNPWNGPNGFSLQIIEVYLDFKDGGNSSAIKMFPDGPGANVNLDPEHPWDVAFRIAGWDYGNLIILPNGTAIQGEMQISADPVKNAIIVKVPKKYIAINEDYGLWGDVLVGSQDGYGPDKWRTAAVDAEQWKLGGADPQAVINGVAPRVIDELVPQGFEPTQEEQLSSYDANDMKLATVKALLLLKQGIVVTDPEGDDHGPGTYTYPTDKVFKPGVFDLLKFKVTEGSDDWTLEFHFKDLGGNPWNGPNGFSLQIIEVYFDFKEGGNVSAIKMFPDGPGSNVRLDPNHPWDLALRIAGWDYGNLIILPDGTAYQGEMQISADPVKNAIIVKVPKKYLNISDYGLYTAVIVGSQDGYGPDKWRPVAAEAEQWKLGGADPQAVIDNLVPRVVDELVPEGFKPTQEEQLSSYDLEKKTLATVLMVPLVNGTGGEEPTPTESPTETTTTTPSETTTTTSTTTGPSSTTTSTPGGGICGPGIIAGLALIPLLLKRRN

SEQ ID NO: 12 - protein, 809 aa, Artificial Sequence
  Hybrid pullulanase of Thermoccus hydrothermalis and Thermococcus litoralis
  SIGNAL (1)..(27)
  mat_peptide (28)..(809)
MKKPLGKIVASTALLISVAFSSSIASAEEPKPLNVIIVWHQHQPYYYDPIQDIYTRPWVRLHAANNYWKMANYLSKYPDVHVAIDLSGSLIAQLADYMNGKKDTYQIVTEKIANGEPLTLEDKWFMLQAPGGFFDHTIPWNGEPVADENGNPYREQWDRYAELKDKRNNAFKKYANLPLNEQKVKITAEFTEQDYIDLAVLFNLAWIDYNYIINTPELKALYDKVDVGGYTKEDVATVLKHQMWLLNHTFEEHEKINYLLGNGNVEVTVVPYAHPIGPLLNDFGWYEDFDAHVKKAHELYKKYLGDNRVEPQGGWAAESALNDKTLEILTNNGWKWVMTDQMVLDILGIPNTVENYYKPWVAEFNGKKIYLFPRNHDLSDRVGFRYSGMNQYQAVEDFVNELLKVQKENYDGSLVYVVTLDGENPWEHYPFDGKIFLEELYKKLTELQKQGLIRTVTPSEYIQMYGDKANKLTPRMMERLDLTGDNVNALLKAQSLGELYDMTGVKEEMQWPESSWIDGTLSTWIGEPQENYGWYWLYMARKALMENKDKMSQADWEKAYEYLLRAEASDWFWWYGSDQDSGQDYTFDRYLKTYLYEMYKLAGVEPPSYLFGNYFPDGEPYTTRGLVGLKDGEMKNFSSMSPLAKGVSVYFDGEGIHFIVKGNLDRFEVSIWEKDERVGNTFTRLQEKPDELSYFMFPFSRDSVGLLITKHVVYENGKAEIYGATDYEKSEKLGEATVKNTSEGIEVVLPFDYIENPSDFYFAVSTVKDGDLEVISTPVELKLPTEVKGVVIADITDPEGDDHGPGNYT

SEQ ID NO: 13 - protein, 412 aa, Pyrococcus furiosus
  mat_peptide (1)..(412): Pyrococcus furiosus protease (Pfu)
AELEGLDESAAQVMATYVWNLGYDGSGITIGIIDTGIDASHPDLQGKVIGWVDFVNGRSYPYDDHGHGTHVASIAAGTGAASNGKYKGMAPGAKLAGIKVLGADGSGSISTIIKGVEWAVDNKDKYGIKVINLSLGSSQSSDGTDALSQAVNAAWDAGLVVVVAAGNSGPNKYTIGSPAAASKVITVGAVDKYDVITSFSSRGPTADGRLKPEVVAPGNWIIAARASGTSMGQPINDYYTAAPGTSMATPHVAGIAALLLQAHPSWTPDKVKTALIETADIVKPDEIADIAYGAGRVNAYKAINYDNYAKLVFTGYVANKGSQTHQFVISGASFVTATLYWDNANSDLDLYLYDPNGNQVDYSYTAYYGFEKVGYYNPTDGTWTIKVVSYSGSANYQVDVVSDGSLSQPGSS

SEQ ID NO: 14 - protein, 595 aa, Penicillium oxalicum
  mat_peptide (1)..(595): mature Penicillium oxalicum glucoamylase sequence
RPDPKGGNLTPFIHKEGERSLQGILDNLGGRGKKTPGTAAGLFIASPNTENPNYYYTWTRDSALTAKCLIDLFEDSRAKFPIDRKYLETGIRDYKSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 15 - protein, 573 aa, Gloeophyllum sepiarium
MYRFLVCALGLAASVLAQSVDSYVSSEGPIAKAGVLANIGPNGSKASGASAGVVVASPSTSDPDYWYTWTRDSSLVFKSLIDQYTTGIDSTSSLRTLIDDFVTAEANLQQVSNPSGTLTTGGLGEPKFNVDETAFTGAWGRPQRDGPALRSTALITYGNWLLSNGNTSYVTSNLWPIIQNDLGYVVSYWNQSTYDLWEEVDSSSFFTTAVQHRALREGAAFATAIGQTSQVSSYTTQADNLLCFLQSYWNPSGGYITANTGGGRSGKDANTLLASIHTYDPSAGCDAATFQPCSDKALSNLKVYVDSFRSVYSINSGVASNAAVATGRYPEDSYQGGNPWYLTTFAVAEQLYDALNVWESQGSLEVTSTSLAFFQQFSSGVTAGTYSSSSSTYSTLTSAIKNFADGFVAINAKYTPSNGGLAEQYSKSDGSPLSAVDLTWSYASALTAFEARNNTQFAGWGAAGLTVPSSCSGNSGGPTVAVTFNVNAETVWGENIYLTGSVDALENWSADNALLLSSANYPTWSITVNLPASTAIEYKYIRKNNGAVTWESDPNNSITTPASGSTTENDTWR

SEQ ID NO: 16 - protein, 583 aa, Rhizomucor pusillus
ATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 17 - protein, 576 aa, Gloephyllum trabeum
  SIGNAL (1)..(17)
  mat_peptide (18)..(576)
MYRFLVCALGLLGTVLAQSVDSYVGSEGPIAKAGVLANIGPNGSKASGAAAGVVVASPSKSDPDYWYTWTRDSSLVFKSLIDQYTTGIDSTSSLRSLIDSFVIAEANIQQVSNPSGTLTTGGLGEPKFNVDETAFTGAWGRPQRDGPALRATALITYGNWLLSNGNTTWVTSTLWPIIQNDLNYVVQYWNQTTFDLWEEVNSSSFFTTAVQHRALREGAAFATKIGQTSSVSSYTTQAANLLCFLQSYWNPTSGYITANTGGGRSGKDANTLLASIHTYDPSAGCDATTFQPCSDKALSNLKVYVDSFRSVYSINSGIASNAAVATGRYPEDSYQGGNPWYLTTFAVAEQLYDALNVWAAQGSLNVTSISLPFFQQFSSSVTAGTYASSSTTYTTLTSAIKSFADGFVAINAQYTPSNGGLAEQFSRSNGAPVSAVDLTWSYASALTAFEARNNTQFAGWGAVGLTVPTSCSSNSGGGGGSTVAVTFNVNAQTVWGENIYITGSVDALSNWSPDNALLLSSANYPTWSITVNLPASTAIQYKYIRKNNGAVTWESDPNNSITTPASGSVTENDTWR

SEQ ID NO: 18 - protein, 573 aa, Pycnoporus sanguineus
MRFTLLASLIGLAVGAFAQSSAVDAYVASESPIAKQGVLNNIGPNGSKAHGAKAGIVVASPSTENPDYLYTWTRDSSLVFKLLIDQFTSGDDTSLRGLIDDFTSAEAILQQVSNPSGTVSTGGLGEPKFNIDETAFTGAWGRPQRDGPALRATSIIRYANWLLDNGNTTYVSNTLWPVIQLDLDYVADNWNQSTFDLWEEVDSSSFFTTAVQHRALREGATFASRIGQSSVVSGYTTQADNLLCFLQSYWNPSGGYVTANTGGGRSGKDSNTVLTSIHTFDPAAGCDAATFQPCSDKALSNLKVYVDAFRSIYTINNGIASNAAVATGRYPEDSYMGGNPWYLTTSAVAEQLYDALYVWDQLGGLNVTSTSLAFFQQFASGLSTGTYSASSSTYATLTSAIRSFADGFLAINAKYTPADGGLAEQYSRNDGTPLSAVDLTWSYAAALTAFAAREGKTYGSWGAAGLTVPASCSGGGGATVAVTFNVQATTVFGENIYITGSVAALQNWSPDNALILSAANYPTWSITVNLPANTVVQYKYIRKFNGQVTWESDPNNQITTPSGGSFTQNDVWR

SEQ ID NO: 19 - protein, 618 aa, Talaromyces emersonii
MASLVAGALCILGLTPAAFARAPVAARATGSLDSFLATETPIALQGVLNNIGPNGADVAGASAGIVVASPSRSDPNYFYSWTRDAALTAKYLVDAFIAGNKDLEQTIQQYISAQAKVQTISNPSGDLSTGGLGEPKFNVNETAFTGPWGRPQRDGPALRATALIAYANYLIDNGEASTADEIIWPIVQNDLSYITQYWNSSTFDLWEEVEGSSFFTTAVQHRALVEGNALATRLNHTCSNCVSQAPQVLCFLQSYWTGSYVLANFGGSGRSGKDVNSILGSIHTFDPAGGCDDSTFQPCSARALANHKVVTDSFRSIYAINSGIAEGSAVAVGRYPEDVYQGGNPWYLATAAAAEQLYDAIYQWKKIGSISITDVSLPFFQDIYPSAAVGTYNSGSTTFNDIISAVQTYGDGYLSIVEKYTPSDGSLTEQFSRTDGTPLSASALTWSYASLLTASARRQSVVPASWGESSASSVPAVCSATSATGPYSTATNTVWPSSGSGSSTTTSSAPCTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ

SEQ ID NO: 20 - protein, 574 aa, Trametes cingulata
MRFTLLTSLLGLALGAFAQSSAADAYVASESPIAKAGVLANIGPSGSKSNGAKAGIVIASPSTSNPNYLYTWTRDSSLVFKALIDQFTTGEDTSLRTLIDEFTSAEAILQQVPNPSGTVSTGGLGEPKFNIDETAFTDAWGRPQRDGPALRATAIITYANWLLDNKNTTYVTNTLWPIIKLDLDYVASNWNQSTFDLWEEINSSSFFTTAVQHRALREGATFANRIGQTSVVSGYTTQANNLLCFLQSYWNPTGGYITANTGGGRSGKDANTVLTSIHTFDPAAGCDAVTFQPCSDKALSNLKVYVDAFRSIYSINSGIASNAAVATGRYPEDSYMGGNPWYLTTSAVAEQLYDALIVWNKLGALNVTSTSLPFFQQFSSGVTVGTYASSSSTFKTLTSAIKTFADGFLAVNAKYTPSNGGLAEQYSRSNGSPVSAVDLTWSYAAALTSFAARSGKTYASWGAAGLTVPTTCSGSGGAGTVAVTFNVQATTVFGENIYITGSVPALQNWSPDNALILSAANYPTWSITVNLPASTTIEYKYIRKFNGAVTWESDPNNSITTPASGTFTQNDTWR

SEQ ID NO: 21 - protein, 483 aa, Bacillus licheniformis [sequence omitted]

SEQ ID NO: 22 - DNA, 25 bp, Artificial Sequence
  Sense Primer
atgcgtctcactctattatcaggtg

SEQ ID NO: 23 - DNA, 39 bp, Artificial Sequence
  Primer F
acacaactggggatccaccatgcgtctcactctattatc

SEQ ID NO: 24 - DNA, 37 bp, Artificial Sequence
  Primer R
agatctcgagaagcttaaaactgccacacgtcgttgg

SEQ ID NO: 25 - DNA, 18 bp, Artificial Sequence
  Primer K79V F
gcagtctttccaattgac

SEQ ID NO: 26 - DNA, 18 bp, Artificial Sequence
  Primer K79V R
aattggaaagactgcccg

SEQ ID NO: 27 - DNA, 39 bp, Artificial Sequence; identical to SEQ ID NO: 23
  Primer F-NP003940

SEQ ID NO: 28 - DNA, 37 bp, Artificial Sequence; identical to SEQ ID NO: 24
  Primer R-NP003940